(19) United States
(12) Patent Application Publication
(10) Pub. No.: US 2019/0141966 A1
(43) Pub. Date: May 16, 2019

(54) Non-Human Animals Comprising SLC30A8 Mutation And Methods Of Use

(71) Applicant: Regeneron Pharmaceuticals, Inc., Tarrytown, NY (US)

(72) Inventors: Sandra Kleiner, Yonkers, NY (US); Jose F. Rojas, Newburgh, NY (US); Jesper Gromada, Scarsdale, NY (US)

(21) Appl. No.: 16/185,567

(22) Filed: Nov. 9, 2018

Related U.S. Application Data
(60) Provisional application No. 62/584,228, filed on Nov. 10, 2017.
(60) Provisional application No. 62/666,337, filed on May 3, 2018.
(60) Provisional application No. 62/689,945, filed on Jun. 26, 2018.

(51) Int. Cl. A01K 67/027; C12N 15/90

(52) U.S. Cl. CPC A01K 67/0275; C12N 15/902; A01K 2207/25; A01K 2227/105; A01K 2217/077; A01K 2217/206; A01K 2267/0362; A01K 2217/05

(57) ABSTRACT

Non-human animal genomes, non-human animal cells, and non-human animals comprising a mutated Slc30a8 locus and methods of making and using such non-human animal genomes, non-human animal cells, and non-human animals are provided. The non-human animals can have increased insulin secretory capacity.

### CROSS-REFERENCE TO RELATED APPLICATIONS

This application claims the benefit of U.S. Application No. 62/584,228, filed Nov. 10, 2017, U.S. Application No. 62/666,337, filed May 3, 2018, and U.S. Application No. 62/689,945, filed Jun. 26, 2018, each of which is herein incorporated by reference in its entirety for all purposes.

### REFERENCE TO A SEQUENCE LISTING SUBMITTED AS A TEXT FILE VIA EFS WEB

The Sequence Listing written in file 523054SEQLIST.txt is 39.2 kilobytes, was created on Nov. 9, 2018, and is hereby incorporated by reference.

### BACKGROUND

Diabetes is a disorder characterized by metabolic defects in insulin-responsive tissues and insulin-producing pancreatic beta cells resulting in a failure to maintain appropriate blood sugar levels in the body. Diabetes in humans can be defined as a disorder corresponding to a fasting plasma glucose concentration greater than 125 mg/dL, or a plasma glucose concentration greater than 199 mg/dL two hours after ingestion of a 75 g oral glucose load. Two major forms of diabetes are type 1 diabetes (T1D) and type 2 diabetes (T2D). T1D is an autoimmune disorder resulting in destruction of beta-pancreatic cells and an absolute deficiency of insulin, the hormone that regulates glucose utilization. By contrast, T2D can occur with normal or even elevated levels of insulin from the inability of tissues to respond appropriately to insulin. Most T2D patients have impaired insulin sensitivity. Insulin secretion cannot compensate for the resistance of peripheral tissues to respond to insulin. Many T2D patients are obese. Type 1.5 diabetes (late autoimmune onset in adults) shows some characteristics of T1D and T2D.

Zinc is required for insulin biosynthesis and processing. Two zinc ions are complexed in a hexameric form of proinsulin, which is ultimately processed to insulin.

SLC30A8 encodes a zinc transporter that is primarily expressed in the pancreatic islet, where it transports zinc into insulin-containing secretory granules. Heterozygous loss-of-function (LOF) mutations in SLC30A8 protect from type 2 diabetes in humans. Several Slc30a8 knockout mouse lines have been characterized, but they do not fully recapitulate the protective human phenotype. Rather, Slc30a8-deficient mice have reduced plasma insulin levels and impaired glucose tolerance.

### SUMMARY

Non-human animals comprising a mutated Slc30a8 locus are provided, as well as methods of making and using such non-human animals. Non-human animal genomes or cells comprising a mutated Slc30a8 locus are also provided.

In one aspect, provided are non-human animal genomes, non-human animal cells, or non-human animals comprising a mutated Slc30a8 locus. Such non-human animal genomes, non-human animal cells, or non-human animals can comprise a mutated Slc30a8 gene, wherein the mutated Slc30a8 gene encodes a truncated SLC30A8 protein and results in the non-human animal having an enhanced capacity for insulin secretion relative to a non-human animal without the mutation.

In one aspect, provided are non-human animals whose genome comprises an endogenous Slc30a8 locus comprising a mutated Slc30a8 gene, wherein the mutated Slc30a8 gene encodes a truncated SLC30A8 protein and results in the non-human animal having an enhanced capacity for insulin secretion relative to a non-human animal without the mutation.

In some such non-human animals, the non-human animals exhibit enhanced capacity for insulin secretion in response to hyperglycemia. Optionally, the non-human animal has increased insulin secretion in response to hyperglycemia induced by insulin receptor inhibition relative to the non-human animal without the mutation. Optionally, the increased insulin secretion in response to hyperglycemia induced by insulin receptor inhibition is not associated with increased beta-cell proliferation or beta-cell mass relative to the non-human animal without the mutation.

In some such non-human animals, the enhanced capacity for insulin secretion is observed when the non-human animals are fed a high-fat diet. Optionally, the non-human animal has increased insulin secretion relative to the non-human animal without the mutation when fed a high-fat diet, wherein the increased insulin secretion is associated with increased beta-cell proliferation or beta-cell mass relative to the non-human animal without the mutation. Optionally, the increased beta-cell proliferation is insulin-receptor-dependent (e.g., dependent on an insulin receptor in the beta cells).

In some such non-human animals, non-human animal cells, or non-human animal genomes, the mutated Slc30a8 gene has a premature termination codon. In some such non-human animals, non-human animal cells, or non-human animal genomes, the mutated Slc30a8 gene comprises a mutation is in the third exon of the Slc30a8 gene. Optionally, the mutation is at the 3′ end of the third exon of the Slc30a8 gene.

In some such non-human animals, non-human animal cells, or non-human animal genomes, the mutated Slc30a8 gene comprises a nonsense mutation. Optionally, the nonsense mutation is in a codon corresponding to the codon encoding R138 in SEQ ID NO: 14 when the SLC30A8 protein encoded by the mutated Slc30a8 gene is optimally aligned with SEQ ID NO: 14. Optionally, the nonsense mutation is at a position corresponding to residue 412 in SEQ ID NO: 21 when the coding sequence of the mutated Slc30a8 gene is optimally aligned with SEQ ID NO: 21.

In some such non-human animals, non-human animal cells, or non-human animal genomes, the mutated Slc30a8 gene is endogenous to the non-human animal. In some such non-human animals, non-human animal cells, or non-human animal genomes, the non-human animal is a non-human mammal. In some such non-human animals, non-human animal cells, or non-human animal genomes, the non-human animal is a rat or a mouse. Optionally, the non-human animal is a mouse. Optionally, the mutated Slc30a8 gene encodes a SLC30A8 protein comprising the sequence set forth in SEQ ID NO: 13. Optionally, the mutated Slc30a8 gene comprises the coding sequence set forth in SEQ ID NO: 22 or degenerates thereof that encode the same amino acid sequence.

Some such non-human animals have increased insulin secretion in response to hyperglycemia induced by insulin receptor inhibition relative to the non-human animal without the mutation. In some such non-human animals, the increased insulin secretion is not associated with increased beta-cell proliferation or beta-cell mass relative to the non-human animal without the mutation. Some such non-human animals have decreased mitochondrial gene expression relative to the non-human animal without the mutation. Some such non-human animals have increased Hvcn1 expression relative to the non-human animal without the mutation. In some such non-human animals, the non-human animal has normal glucose homeostasis and glucose-induced insulin secretion on a control chow diet relative to the non-human animal without the mutation. In some such non-human animals, the non-human animal has a normal metabolic phenotype on a control chow diet relative to the non-human animal without the mutation.

Some such non-human animals have one or more of the following characteristics relative to the non-human animal without the mutation: (a) increased glucose-induced insulin secretion when fed the high-fat diet; (b) increased pancreatic beta-cell proliferation when fed the high-fat diet; (c) increased number of pancreatic beta cells when fed the high-fat diet; and (d) increased fed plasma insulin levels after blockade of the insulin receptor. Optionally, the non-human animal has all of the following characteristics relative to the non-human animal without the mutation: (a) increased glucose-induced insulin secretion when fed the high-fat diet; (b) increased pancreatic beta-cell proliferation when fed the high-fat diet; (c) increased number of pancreatic beta cells when fed the high-fat diet; and (d) increased fed plasma insulin levels after blockade of the insulin receptor.

Some such non-human animals have one or more of the following characteristics relative to the non-human animal without the mutation: (a) increased circulating insulin levels after fed the high-fat diet for 20 weeks; (b) increased number of pancreatic beta cells after fed the high-fat diet for 20 weeks; (c) decreased proinsulin-to-insulin ratio when fed the high-fat diet; and (d) increased fed plasma insulin levels after blockade of the insulin receptor. Optionally, the non-human animal has all of the following characteristics relative to the non-human animal without the mutation: (a) increased circulating insulin levels after fed the high-fat diet for 20 weeks; (b) increased number of pancreatic beta cells after fed the high-fat diet for 20 weeks; (c) decreased proinsulin-to-insulin ratio when fed the high-fat diet; and (d) increased fed plasma insulin levels after blockade of the insulin receptor.

In some such non-human animals, Slc30a8 mRNA expression levels in the islets of the non-human animal are at least 25% of Slc30a8 mRNA expression levels in the islets of the non-human animal without the mutation.

Some such non-human animals, non-human animal cells, or non-human animal genomes are heterozygous for the mutation. Some such non-human animals, non-human animal cells, or non-human animal genomes are homozygous for the mutation. Some such non-human animals, non-human animal cells, or non-human animal genomes are male. Some such non-human animals, non-human animal cells, or non-human animal genomes are female.

In another aspect, provided are methods for making any of the above non-human animals. Some such methods comprise: (a) contacting the genome of a non-human animal pluripotent cell that is not a one-cell stage embryo with: (i) an exogenous repair template comprising an insert nucleic acid flanked by a 5′ homology arm that hybridizes to a 5′ target sequence at the Slc30a8 locus and a 3′ homology arm that hybridizes to a 3′ target sequence at the Slc30a8 locus, wherein the insert nucleic acid comprises the mutation; and (ii) a Cas9 protein; and (iii) a guide RNA that hybridizes to a guide RNA recognition sequence within the Slc30a8 locus, wherein the Slc30a8 gene is modified to comprise the mutation; and (b) introducing the modified non-human animal pluripotent cell into a host embryo; and (c) implanting the host embryo into a surrogate mother to produce a genetically modified F0 generation non-human animal in which the Slc30a8 gene is modified to comprise the mutation, wherein the mutation results in the F0 generation non-human animal having an enhanced capacity for insulin secretion relative to a non-human animal without the mutation when fed the high-fat diet. Some such methods comprise: (a) contacting the genome of a non-human animal pluripotent cell that is not a one-cell stage embryo with: (i) an exogenous repair template comprising an insert nucleic acid flanked by a 5′ homology arm that hybridizes to a 5′ target sequence at the Slc30a8 locus and a 3′ homology arm that hybridizes to a 3′ target sequence at the Slc30a8 locus, wherein the insert nucleic acid comprises the mutation; and (ii) a Cas9 protein; and (iii) a guide RNA that hybridizes to a guide RNA recognition sequence within the Slc30a8 locus, wherein the Slc30a8 gene is modified to comprise the mutation; and (b) introducing the modified non-human animal pluripotent cell into a host embryo; and (c) gestating the host embryo in a surrogate mother to produce a genetically modified F0 generation non-human animal in which the Slc30a8 gene is modified to comprise the mutation, wherein the mutation results in the F0 generation non-human animal having an enhanced capacity for insulin secretion relative to a non-human animal without the mutation when fed the high-fat diet. Optionally, the pluripotent cell is an embryonic stem cell. Optionally, the exogenous repair template is a large targeting vector that is at least 10 kb in length, or wherein the exogenous repair template is a large targeting vector in which the sum total of the 5′ homology arm and the 3′ homology arm is at least 10 kb in length.

Some such methods comprise: (a) contacting the genome of a non-human animal one-cell stage embryo with: (i) an exogenous repair template comprising an insert nucleic acid flanked by a 5′ homology arm that hybridizes to a 5′ target sequence at the Slc30a8 locus and a 3′ homology arm that hybridizes to a 3′ target sequence at the Slc30a8 locus, wherein the insert nucleic acid comprises the mutation; (ii) a Cas9 protein; and (iii) a guide RNA that that hybridizes to a guide RNA recognition sequence within the Slc30a8 locus, wherein the Slc30a8 gene is modified to comprise the mutation; and (b) implanting the modified non-human animal one-cell stage embryo into a surrogate mother to produce a genetically modified F0 generation non-human animal in which the Slc30a8 gene is modified to comprise the mutation, wherein the mutation results in the F0 generation non-human animal having an enhanced capacity for insulin secretion relative to a non-human animal without the mutation when fed the high-fat diet. Some such methods comprise: (a) contacting the genome of a non-human animal one-cell stage embryo with: (i) an exogenous repair template comprising an insert nucleic acid flanked by a 5′ homology arm that hybridizes to a 5′ target sequence at the Slc30a8 locus and a 3′ homology arm that hybridizes to a 3′ target sequence at the Slc30a8 locus, wherein the insert nucleic acid comprises the mutation; (ii) a Cas9 protein; and (iii) a guide RNA that that hybridizes to a guide RNA recognition sequence within the Slc30a8 locus, wherein the Slc30a8 gene is modified to comprise the mutation; and (b) gestating the modified non-human animal one-cell stage embryo in a surrogate mother to produce a genetically modified F0 generation non-human animal in which the Slc30a8 gene is modified to comprise the mutation, wherein the mutation results in the F0 generation non-human animal having an enhanced capacity for insulin secretion relative to a non-human animal without the mutation when fed the high-fat diet.

In some such methods, step (a) further comprises contacting the genome of the non-human animal pluripotent cell or the non-human one-cell stage embryo with a second guide RNA that hybridizes to a second guide RNA recognition sequence within the Slc30a8 locus.

In some such methods, the exogenous repair template a single-stranded oligodeoxynucleotide. Optionally, the exogenous repair template is between about 80 nucleotides to about 200 nucleotides in length.

In another aspect, provided are methods of screening compounds for activity for ameliorating or exacerbating type-2-diabetes. Some such methods comprise: (a) contacting any of the above subject non-human animals with the compound; and (b) measuring one or more of the following in the subject non-human animal relative to a control non-human animal not contacted with the compound, wherein the control non-human animal comprises the same Slc30a8 mutation as the subject non-human animal: (1) glucose-induced insulin secretion when fed the high-fat diet; (2) pancreatic beta-cell proliferation levels when fed the high-fat diet; (3) number of pancreatic beta cells when fed the high-fat diet; and (4) fed plasma insulin levels after blockade of the insulin receptor, whereby activity for ameliorating type 2 diabetes is identified by one or more of the following in the subject non-human animal compared with the control non-human animal: (1) increased glucose-induced insulin secretion when fed the high-fat diet; (2) increased pancreatic beta-cell proliferation when fed the high-fat diet; (3) increased number of pancreatic beta cells when fed the high-fat diet; and (4) increased fed plasma insulin levels after blockade of the insulin receptor, and whereby activity for exacerbating type 2 diabetes is identified by one or more of the following in the subject non-human animal compared with the control non-human animal: (1) decreased glucose-induced insulin secretion when fed the high-fat diet; (2) decreased pancreatic beta-cell proliferation when fed the high-fat diet; (3) decreased number of pancreatic beta cells when fed the high-fat diet; and (4) decreased fed plasma insulin levels after blockade of the insulin receptor.

Some such methods comprise: (a) contacting any of the above subject non-human animals with the compound; and (b) measuring one or more of the following in the subject non-human animal relative to a control non-human animal not contacted with the compound, wherein the control non-human animal comprises the same Slc30a8 mutation as the subject non-human animal: (1) capacity to secrete insulin in response to hyperglycemia; (2) insulin clearance; (3) mitochondrial gene expression; and (4) Hvcn1 expression, whereby activity for ameliorating type 2 diabetes is identified by one or more of the following in the subject non-human animal compared with the control non-human animal: (1) increased capacity to secrete insulin in response to hyperglycemia; (2) increased insulin clearance; (3) decreased mitochondrial gene expression; and (4) increased Hvcn1 expression, and whereby activity for exacerbating type 2 diabetes is identified by one or more of the following in the subject non-human animal compared with the control non-human animal: (1) decreased capacity to secrete insulin in response to hyperglycemia; (2) decreased insulin clearance; (3) increased mitochondrial gene expression; and (4) decreased Hvcn1 expression.

In another aspect, provided are non-human animal cells whose genome comprises an endogenous Slc30a8 locus comprising a mutated Slc30a8 gene, wherein the mutated Slc30a8 gene encodes a truncated SLC30A8 protein, and wherein a non-human animal comprising the mutated Slc30a8 gene has an enhanced capacity for insulin secretion relative to a non-human animal without the mutation.

In another aspect, provided are non-human animal genomes comprising an endogenous Slc30a8 locus comprising a mutated Slc30a8 gene, wherein the mutated Slc30a8 gene encodes a truncated SLC30A8 protein, and wherein a non-human animal comprising the mutated Slc30a8 gene has an enhanced capacity for insulin secretion relative to a non-human animal without the mutation.

In another aspect, provided are targeting vectors for generating a mutated Slc30a8 gene at an endogenous Slc30a8 locus in a non-human animal, wherein the targeting vector comprises a 5′ homology arm targeting a 5′ target sequence at the endogenous Slc30a8 locus and a 3′ homology arm targeting a 3′ target sequence at the endogenous Slc30a8 locus, wherein the targeting vector comprises a mutation in the Slc30a8 gene, wherein the mutated Slc30a8 gene encodes a truncated SLC30A8 protein, and wherein a non-human animal comprising the mutated Slc30a8 gene has an enhanced capacity for insulin secretion relative to a non-human animal without the mutation.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1D show analysis of Slc30a8 RNA and protein in islets from male R138X mice on chow diet. FIG. 1A shows Slc30a8 RNA in situ hybridization of pancreatic islets isolated from wild type (WT), homozygous knockout (KO), and homozygous R138X mice. KO islets were used as negative control. Red: glucagon RNA, green: insulin RNA, white: Slc30a8 RNA. FIG. 1B shows quantification of islet Slc30a8 RNA levels using TAQMAN® analysis. FIG. 1C shows Western blot of islets isolated from chow-fed WT, KO and R138X mice. KO islets were used as negative control. Arrow: SLC30A8 protein; * denotes unspecific bands. FIG. 1D shows dithizone staining of pancreatic islets isolated from WT, KO, and R138X mice.

FIGS. 2A-2P show the metabolic phenotype of male homozygous R138X mice on chow diet. FIGS. 2A-2D and 2L show body weight (FIG. 2L), blood glucose (FIG. 2A), plasma insulin (FIG. 2B), proinsulin (FIG. 2C), and C-peptide (FIG. 2D) levels in fed and fasted WT and R138X mice. FIGS. 2E-2F show proinsulin/insulin ratio (FIG. 2E) and C-peptide/insulin ratio (FIG. 2F) in fed and fasted WT and R138X mice. FIG. 2G shows oral glucose tolerance test in WT and R138X mice. Data are displayed as blood glucose over time. FIG. 2H shows plasma insulin levels in WT or R138X mice after an overnight fast (time 0) and at the indicated times after an intraperitoneal injection of glucose. Data are displayed as blood glucose levels over time. FIG. 2I shows histology for insulin in pancreas isolated from WT and R138X mice. FIG. 2J shows quantification of pancreatic insulin staining. FIG. 2K shows quantification of islet number. Values represent the means±SEM (n=6-20 mice per genotype). FIG. 2M shows results of an insulin tolerance test in 4 hr fasted WT and R138X mice. Data are displayed as blood glucose levels over time (n=6/genotype). FIG. 2N shows further quantification of pancreatic insulin staining (islet mass; n=6/genotype). Values represent the means±SEM. FIGS. 2O-2P show proinsulin/C-peptide ratio (FIG. 2O) and insulin/C-peptide ratio (FIG. 2P) in fed and fasted WT and R138X mice on a chow diet. N=18-20 mice per genotype.

FIGS. 3A-3M show the metabolic phenotype of male homozygous R138X mice on HFD diet. FIGS. 3A-3F show blood glucose (FIG. 3A), plasma insulin (FIG. 3B), proinsulin (FIG. 3C), and C-peptide (FIG. 3D) levels in fed and fasted WT and R138X mice. FIGS. 3E-3F show proinsulin/insulin ratio (FIG. 3E) and C-peptide/insulin ratio (FIG. 3F) in fed and fasted WT and R138X mice. FIG. 3G shows oral glucose tolerance test in WT and R138X mice. Data are displayed as blood glucose over time. Area under the curve is shown on the right. FIG. 3H shows blood insulin levels in WT or R138X mice after an overnight fast (time 0) and at the indicated times after an intraperitoneal injection of glucose. Data are displayed as blood glucose levels over time. FIG. 3I shows histology for insulin in pancreas isolated from WT and R138X mice. FIG. 3J shows quantification of pancreatic insulin staining. FIG. 3K shows quantification of islet number. FIG. 3L shows immunohistochemistry for Ki67 (green) and insulin (red). FIG. 3M shows quantification of Ki67 and insulin double-positive cells. Values represent the means±SEM (n=4-17 mice per genotype).

FIGS. 4A-4K show metabolic phenotype of male homozygous Slc30a8 KO mice on HFD diet. FIGS. 4A-4D show blood glucose (FIG. 4A), plasma insulin (FIG. 4B), proinsulin (FIG. 4C), and C-peptide (FIG. 4D) levels in fed and fasted WT and Slc30a8 KO mice. FIGS. 4E-4F show proinsulin/insulin ratio (FIG. 4E) and C-peptide/insulin ratio (FIG. 4F) in fed and fasted WT and Slc30a8 KO mice. FIG. 4G shows oral glucose tolerance test in WT and Slc30a8 KO mice. Data are displayed as blood glucose levels over time. FIG. 4H shows blood insulin levels in WT or Slc30a8 KO mice after an overnight fast (time 0) and at the indicated times after an intraperitoneal injection of glucose. Data are displayed as glucose levels over time. FIG. 4I shows histology for insulin in pancreas isolated from WT and Slc30a8 KO mice. FIG. 4J shows quantification of pancreatic insulin staining. FIG. 4K shows quantification of islet number. Values represent the means±SEM (n=6-17 mice per genotype).

FIG. 5 shows in vitro expression and stabilization of human R138X protein by proteasomal inhibition. Western blot analysis of HEK293 lysates is shown after overexpression of either myc-tagged SLC30A8 WT or R138X protein alone or together. Cells were treated for 6 hr with indicated compounds and probed with the respective antibodies.

FIGS. 6A-6E show glucagon histology in chow and HFD conditions is unchanged in male homozygous R138X mice. FIG. 6A shows histology for glucagon in pancreas isolated from chow-fed WT and R138X mice. FIGS. 6B and 6E show quantification of pancreatic glucagon staining shown in FIG. 6A. Values represent the means±SEM (n=6 per genotype). FIG. 6C shows histology for glucagon in pancreas isolated from HFD-fed WT and R138X mice. FIG. 6D shows quantification of pancreatic glucagon staining shown in FIG. 6C (n=4-17 mice per genotype).

FIGS. 7A-7K show metabolic phenotype of male homozygous Slc30a8 KO mice on chow diet. FIGS. 7A-7D show blood glucose (FIG. 7A), plasma insulin (FIG. 7B), proinsulin (FIG. 7C), and C-peptide (FIG. 7D) levels in fed and fasted WT and Slc30a8 KO mice. FIGS. 7E-7F show proinsulin/insulin ratio (FIG. 7E) and C-peptide/insulin ratio (FIG. 7F) in fed and fasted WT and Slc30a8 KO mice. FIG. 7G shows oral glucose tolerance test in WT and Slc30a8 KO mice. Data are displayed as blood glucose levels over time. FIG. 7H shows plasma insulin levels in WT or Slc30a8 KO mice after an overnight fast (time 0) and at the indicated times after an intraperitoneal injection of glucose. Data are displayed as blood glucose levels over time. FIG. 7I shows histology for insulin in pancreas isolated from WT and Slc30a8 KO mice. FIG. 7J shows quantification of pancreatic insulin staining. FIG. 7K shows quantification of islet number. Values represent the means±SEM (n=6-10 mice per genotype).

FIGS. 8A-8K show metabolic phenotype of female homozygous R138X mice on HFD. FIGS. 8A-8D show blood glucose (FIG. 8A), plasma insulin (FIG. 8B), proinsulin (FIG. 8C), and C-peptide (FIG. 8D) levels in fed and fasted WT and R138X mice. FIGS. 8E-8F show proinsulin/insulin ratio (FIG. 8E) and C-peptide/insulin ratio (FIG. 8F) in fed and fasted WT and R138X mice. FIG. 8G shows oral glucose tolerance test in WT and R138X mice. Data are displayed as glucose levels over time. FIG. 8H shows plasma insulin levels in WT or R138X mice after an overnight fast (time 0) and at the indicated times after an intraperitoneal injection of glucose. Data are displayed as glucose levels over time. FIG. 8I shows histology for insulin in pancreas isolated from WT and R138X mice. FIG. 8J shows quantification of pancreatic insulin staining. FIG. 8K shows quantification of islet number. Values represent the means±SEM (n=6-9 mice per genotype).

FIGS. 9A-9M show heterozygous R138X (HET) mice on HFD have higher glucose-stimulated insulin levels. FIG. 9A shows Western blot of islets isolated from chow-fed WT, heterozygous R138X mice. Arrow: SLC30A8 protein; * denotes unspecific bands. FIG. 9B shows dithizone staining of pancreatic islets isolated from WT and heterozygous R138X mice. FIGS. 9C-9F show blood glucose (FIG. 9C), plasma insulin (FIG. 9D), proinsulin (FIG. 9E), and C-peptide (FIG. 9F) levels in fed and fasted WT and R138X HET mice. FIGS. 9G-9H show proinsulin/insulin ratio (FIG. 9G) and C-peptide/insulin ratio (FIG. 9H) in fed and fasted WT and HET mice. FIG. 9I shows oral glucose tolerance test in WT and R138X HET mice. Data are displayed as blood glucose levels over time. FIG. 9J shows plasma insulin levels in WT or R138X HET mice after an overnight fast (time 0) and at the indicated times after an intraperitoneal injection of glucose. Data are displayed as glucose levels over time. FIG. 9K shows histology for insulin in pancreas isolated from WT and R138X HET mice. FIG. 9L shows quantification of pancreatic insulin staining. FIG. 9M shows quantification of islet number. Values represent the means±SEM (n=6-9 mice per genotype).

FIGS. 10A-10C show schematics for targeting the mouse Slc30a8 locus to generate R138X mice. FIG. 10A shows the wild type mouse locus and indicates the location of the pArg137* C>T point mutation and the 29 bp deletion that are generated through targeting. An allele-specific screening assay (8084 AS) and a TAQMAN® downstream screening assay (8084 TD) are shown. FIG. 10B shows a schematic of the targeted locus. FIG. 10C shows a schematic of the targeted locus after self-excising of the self-deleting drug selection cassette.

FIG. 11 shows a protein alignment of the wild type mouse SLC30A8 protein (SEQ ID NO: 12), the expected truncated SLC30A8 Arg137* protein in R138X mice (SEQ ID NO: 13), and the wild type human SLC30A8 protein (SEQ ID NO: 14).

FIG. 12 shows fed plasma glucose levels and fed plasma insulin levels in WT (closed symbols) and homozygous R138X mice (open symbols) treated with either S961 (dashed lines) or PBS (solid lines) using a constant minipump infusion. Fed plasma glucose levels and insulin levels were measured at the indicated times throughout the experiment (21 days).

FIGS. 13A-13I show that R138X mice secrete more insulin under chronic hyperglycemia caused by the insulin receptor antagonist S961. FIG. 13A shows nonfasted plasma glucose levels in R138X and WT mice continuously treated with the insulin receptor antagonist S961 (20 nMol/week) or PBS for 22 days. FIG. 13B shows nonfasted plasma insulin on days 0, 4, 19 and 22 R138X and WT mice treated with S961 (20 nMol/week) or PBS. FIG. 13C shows plasma active GLP-1 levels in WT and R138X mice after 22 days of treatment. FIGS. 13D and 13E show fed and fasted glucose (FIG. 13D) and insulin (FIG. 13E) levels measured day 19 and 20 after initiation of treatment. FIG. 13F shows immunohistochemistry for KI-67 (white), insulin (green), and glucagon (red). FIG. 13G shows quantification of KI-67 and insulin double-positive cells. FIG. 13H shows quantification of pancreatic insulin staining shown in (FIG. 13I). FIG. 13I shows histology for insulin in pancreas isolated from WT and R138X mice after 22 days of treatment. Values represent the means±SEM (n=5-7 mice per treatment and genotype).

FIGS. 14A-14G show hormone levels and glucagon histology after 22 days of insulin receptor antagonist S961 (20 nMol/week) or PBS S961 treatment. Insulin (FIG. 14A), Proinsulin (FIG. 14B), C-peptide (FIG. 14C), Proinsulin/C-peptide ratio (FIG. 14D), Insulin/C-peptide ratio (FIG. 14E) in fed and fasted WT and R138X mice after 22 days of treatment. FIG. 14F shows quantification of pancreatic glucagon staining shown in FIG. 14G. FIG. 14G shows histology for glucagon in pancreas isolated from WT and R138X mice after 22 days of treatment. Values represent the means±SEM (n=5-7 mice per genotype).

FIG. 15 shows gene expression of Slc30a8, expressed in RPKM (reads per kilobase million).

FIGS. 16A-16C show gene expression changes in islets from WT versus R138X mice. FIG. 16A shows RPKM values for insulin 2 (Ins2) and insulin 1 (Ins1). FIG. 16B shows RPKM values for beta-cell regulators. FIG. 16C shows RPKM values for Hvcn1.

FIG. 17 shows circulatory zinc levels in fed and fasted male WT and homozygous R138X mice after 20 weeks on high-fat diet (HFD). The mice were approximately 29 weeks of age. 12 WT mice were assessed, and 13 R138X mice were assessed.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “targeting vector” refers to a recombinant nucleic acid that can be introduced by homologous recombination, non-homologous-end-joining-mediated ligation, or any other means of recombination to a target position in the genome of a cell.

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or non-human animal. For example, an endogenous Slc30a8 sequence of a non-human animal refers to a native Slc30a8 sequence that naturally occurs at the Slc30a8 locus in the non-human animal. The term “endogenous locus” refers to a location on a chromosome at which a particular genetic element is naturally found. For example, an endogenous Slc30a8 locus of a non-human animal refers to the naturally occurring Slc30a8 locus in the non-human animal (i.e., the region of the non-human animal genome at which the Slc30a8 gene is naturally found). As an example, the endogenous mouse Slc30a8 locus maps to chromosome 15 (NCBI RefSeq GeneID 239436; Assembly GRCm38.p4; location NC_000081.6 (52295553-52335733)). The term “endogenous Slc30a8 locus” of a non-human animal does not refer to a Slc30a8 gene randomly inserted into the genome of the non-human animal or inserted at a region other than the region of the non-human animal genome at which the Slc30a8 gene is naturally found. Likewise, the term “endogenous Slc30a8 locus” of a non-human animal does not refer to a Slc30a8 gene located, for example, in cells (e.g., human beta cells) grafted into the non-human animal.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, a “Slc30a8 locus” may refer to the specific location of a Slc30a8 gene, Slc30a8 DNA sequence, SLC30a8-encoding sequence, or Slc30a8 position on a chromosome of the genome of an organism that has been identified as to where such a sequence resides. A “Slc30a8 locus” may comprise a regulatory element of a Slc30a8 gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

The “coding region” or “coding sequence” of a gene consists of the portion of a gene's DNA or RNA, composed of exons, that codes for a protein. The region begins at the start codon on the 5′ end and ends at the stop codon on the 3′ end.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “variant” refers to a nucleotide sequence differing from the sequence most prevalent in a population (e.g., by one nucleotide) or a protein sequence different from the sequence most prevalent in a population (e.g., by one amino acid).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

The term “reporter gene” refers to a nucleic acid having a sequence encoding a gene product (typically an enzyme) that is easily and quantifiably assayed when a construct comprising the reporter gene sequence operably linked to an endogenous or heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreenl), yellow fluorescent proteins (e.g., YFP, eYFP, Citrine, Venus, YPet, PhiYFP, and ZsYellowl), blue fluorescent proteins (e.g., BFP, eBFP, eBFP2, Azurite, mKalamal, GFPuv, Sapphire, and T-sapphire), cyan fluorescent proteins (e.g., CFP, eCFP, Cerulean, CyPet, AmCyanl, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

Repair in response to double-strand breaks (DSBs) occurs principally through two conserved DNA repair pathways: homologous recombination (HR) and non-homologous end joining (NHEJ). See Kasparek & Humphrey (2011) Seminars in Cell & Dev. Biol. 22:886-897, herein incorporated by reference in its entirety for all purposes. Likewise, repair of a target nucleic acid mediated by an exogenous donor nucleic acid can include any process of exchange of genetic information between the two polynucleotides.

The term “recombination” includes any process of exchange of genetic information between two polynucleotides and can occur by any mechanism. Recombination can occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLOS ONE 7:e45768:1-9; and Wang et al. (2013) Nat Biotechnol. 31:530-532, each of which is herein incorporated by reference in its entirety for all purposes.

NHEJ includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23(3):539-546, each of which is herein incorporated by reference in its entirety for all purposes. If blunt ends are ligated, target and/or donor resection may be needed to generation regions of microhomology needed for fragment joining, which may create unwanted alterations in the target sequence.

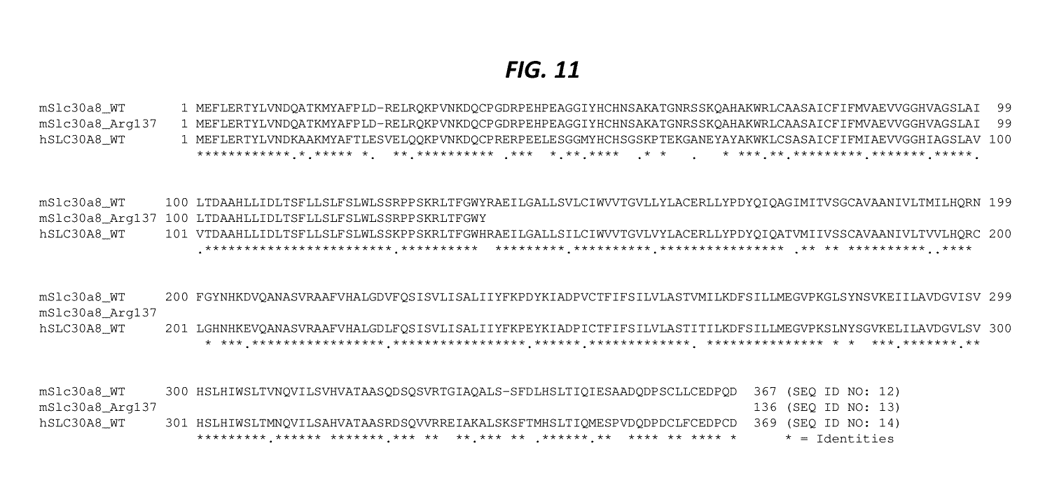

The term “R138X mice” refers to mice carrying a mutation in the mouse Slc30a8 locus corresponding to the human SLC30A8 putative LOF mutation, R138X. The mutation in the mouse Slc30a8 locus is c.409C>T (transcript accession number NM_172816.3), p.Arg137X, meaning a C to T substitution at nucleotide 409 of the coding sequence resulting in the codon encoding amino acid residue 137 being mutated from a codon encoding an arginine residue to a stop codon (R137*^{stop}). The corresponding human SLC30A8 mutation is c.412 C>T (transcript accession number NM_173851), p.Arg138X, meaning a C to T substitution at nucleotide 412 of the coding sequence resulting in the codon encoding amino acid residue 138 being mutated from a codon encoding an arginine residue to a stop codon (R138*^{stop}). Amino acid residue 137 of the mouse SLC30A8 protein corresponds with amino acid residue 138 of the human SLC30A8 protein when the two are optimally aligned. See FIG. 11. Although the mutation in the mouse Slc30a8 gene is in the codon encoding amino acid residue 137 of the mouse SLC30A8 protein, these mice are referred to herein as “R138X mice” in reference to the corresponding human SLC30A8 mutation.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

Unless otherwise apparent from the context, the term “about” encompasses values within a standard margin of error of measurement (e.g., SEM) of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Disclosed herein are non-human animal genomes, non-human animal cells, and non-human animals comprising in their genome a mutated Slc30a8 locus and methods of using such non-human animal cells and non-human animals. The zinc transporter SLC30A8 is primarily expressed in the islets of the endocrine pancreas. SLC30A8 is only one of three genes to date for which putative loss-of-function mutations have been shown to protect from development of type 2 diabetes. This is an interesting but also puzzling observation since mouse models deficient in Slc30a8 have mildly impaired glucose homeostasis. We have generated a mouse model comprising a mutated Slc30a8 locus mimicking a protective human SLC30A8 allele. This mouse model can for the first time explain why humans are protected from type 2 diabetes. The mice have increased insulin secretory capacity. In particular, the mice have higher glucose-induced insulin secretion, which may be mediated in part by an increased number of pancreatic beta cells. These SLC30A8 mutation data support the notion that beta-cell expansion is a valid therapeutic strategy to manage type 2 diabetes.

The cells and non-human animal models disclosed herein can be used for drug screening as well as to provide insights into the mechanism of T2D (e.g., the mechanism by which SLC30A8 contributes to insulin processing and diabetes) and potentially new therapeutic and diagnostic targets.

### II. Non-Human Animals Comprising a Mutated Slc30a8 Locus

The cells and non-human animals disclosed herein comprise (e.g., in their genome) a mutation (e.g., a mutation that does not naturally occur in non-human animals) in the Slc30a8 locus. The mutated Slc30a8 locus can have a premature termination codon and/or can encode a truncated SLC30A8 protein. The non-human animals comprising a mutated Slc30a8 locus have an enhanced capacity for insulin secretion when fed a high-fat diet relative to a non-human animal without the mutation when fed the same diet.

A. Slc30a8

The cells and non-human animals described herein comprise a mutated Slc30a8 (solute carrier family 30 member 8) locus. Other names for SLC30A8 include zinc transporter 8, ZnT-8, and Znt8. The protein encoded by Slc30a8 is a zinc transporter involved in the accumulation of zinc in intracellular vesicles. Slc30a8 is expressed at a high level in the pancreas, particularly in islets of Langerhans. The encoded protein co-localizes with insulin in the secretory pathway granules of the insulin-secreting INS-1 cells.

Human SLC30A8 maps to human 8q24.11 on chromosome 8 (NCBI RefSeq GeneID 169026; Assembly GRCh38.p7; location 116950273-117176714). The gene has been reported to have eight exons and seven introns. However, isoforms with more than eight exons (including non-coding exons) are also possible. The wild type human SLC30A8 protein has been assigned UniProt accession number Q8IWU4. Two isoforms are known, Q8IWU4 isoform 1 (SEQ ID NO: 14) and Q8IWU4 isoform 2. The mRNA encoding isoform 1 is assigned NCBI RefSeq NM_173851.2 (SEQ ID NO: 17). The full-length human protein has 369 amino acids including six transmembrane regions, four intracellular topological domains, and three extracellular topological domains. Delineations between these domains are as designated in UniProt. Reference to human SCL30A8 includes the canonical (wild type) forms as well as all allelic forms and isoforms. Any other forms of human SLC30A8 have amino acids numbered for maximal alignment with the wild type form, aligned amino acids being designated the same number.

Mouse Slc30a8 maps to chromosome 15 (NCBI RefSeq GeneID 239436; Assembly GRCm38.p4; location NC_000081.6 (52295553-52335733)). The gene has been reported to have eight exons and seven introns. The wild type mouse SLC30A8 protein has been assigned UniProt accession number Q8BGG0 (SEQ ID NO: 12). The mRNA encoding mouse SLC30A8 is assigned NCBI RefSeq NM_172816.3 (SEQ ID NO: 16). The full-length mouse protein has 367 amino acids including six transmembrane regions, four intracellular topological domains, and three extracellular topological domains. Delineations between these domains are as designated in UniProt. Reference to mouse SCL30A8 includes the canonical (wild type) forms, as well as all allelic forms and isoforms. Any other forms of mouse SLC30A8 have amino acids numbered for maximal alignment with the wild type form, aligned amino acids being designated the same number.

An exemplary rat SLC30A8 protein is designated by UniProt Accession Number P0CE46.

Polymorphisms in the human SLC30A8 gene are associated with altered risk of type 2 diabetes (T2D). See, e.g., Sladek et al. (2007) Nature 445(7130):881-885 and Davidson et al. (2014) Trends Endocrinol. Metab. 25(8):415-424, each of which is herein incorporated by reference in its entirety for all purposes. Some such polymorphisms or mutations in humans can reduce risk of T2D or are protective against T2D in humans. A polymorphism or mutation in the SLC30A8 gene that reduces risk of T2D includes a polymorphism or mutation that confers protection against T2D, that confers resistance to T2D, or that is associated with protection against or resistance to T2D. For example, various stop codon mutations, frameshift mutations, splice site mutations, or initiator codon variations that cause protein truncation in human SLC30A8 are protective against T2D when heterozygous. One of these is designated c.412 C>T (transcript accession number NM_173851), p.Arg138X meaning a C to T substitution at nucleotide 412 of the coding sequence resulting in the codon encoding amino acid residue 138 being mutated from a codon encoding an arginine residue to a stop codon (R138*^{stop}). See, e.g., Flannick et al. (2014) Nat. Genet. 46(4):357-363, herein incorporated by reference in its entirety for all purposes. These phenotypes have not been replicated in previous animal models but are replicated in the non-human animals disclosed herein.

B. Mutated Slc30a8 Loci

The mutated Slc30a8 loci disclosed herein result in non-human animals having an enhanced capacity for insulin secretion when fed a high-fat diet relative to a non-human animal without the mutation when fed the same diet. The mutated Slc30a8 loci may reduce T2D risk. Non-human animals comprising a mutated Slc30a8 locus can have one or more or all of the following characteristics relative to the non-human animal without the mutation (e.g., a wild type non-human animal): (1) increased glucose-induced insulin secretion when fed a high-fat diet; (2) increased pancreatic beta-cell proliferation when fed a high-fat diet; (3) increased number of pancreatic beta cells when fed a high-fat diet; and (4) increased fed plasma insulin levels after blockade of the insulin receptor (e.g., with S961). Additionally or alternatively, non-human animals comprising a mutated Slc30a8 locus can have one or more or all of the following characteristics relative to the non-human animal without the mutation (e.g., a wild type non-human animal): (1) increased circulating insulin levels after 20, 25, 30, 35, 40, 45, or 50 weeks (e.g., after 20 or 30 weeks) being fed on a high-fat diet; (2) increased number of pancreatic beta cells after 20, 25, 30, 35, 40, 45, or 50 weeks (e.g., after 20 or 30 weeks) being fed on a high-fat diet; (3) decrease in the proinsulin-to-insulin ratio when fed a high-fat diet; (4) increased fed plasma insulin levels after blockade of the insulin receptor (e.g., after 1, 2, 3, 4, 5, 6, 7, 8, 9, 10, 11, 12, 13, 14, 15, 16, 17, 18, 19, 20, 21, 22, 23, 24, or 25 days); and (5) pancreatic beta cells with improved insulin processing and better health (e.g., as evidenced by decrease in ratio of proinsulin-to-insulin when fed a high-fat diet). Additionally or alternatively, non-human animals comprising a mutated Slc30a8 locus can have significant Slc30a8 mRNA expression in islets (e.g., at least 5%, 10%, 15%, 20%, 25%, 30%, 35%, 40%, 45%, 50%, 55%, 60%, 65%, 70%, 75%, 80%, 85%, 90%, 95%, or 100% of wild type levels, such as at least 25% or 30% of wild type levels).

Compared to non-human animals without the mutation (e.g., wild type non-human animals), non-human animals with a mutated Slc30a8 locus can have a normal metabolic phenotype when fed a control chow diet. Compared to non-human animals without the mutation (e.g., wild type non-human animals), non-human animals with a mutated Slc30a8 locus can have normal glucose homeostasis and/or normal glucose-induced insulin secretion when fed a control chow diet. Compared to non-human animals without the mutation (e.g., wild type non-human animals), non-human animals with a mutated Slc30a8 locus can also have one or more or all of the following characteristics when fed a control chow diet: do not differ significantly in body weight, do not differ significantly in circulating blood glucose levels, do not differ significantly in circulating insulin levels, do not differ significantly in circulating proinsulin levels, do not differ significantly in circulating C-peptide levels, do not differ significantly in the ratio of proinsulin/C-peptide, do not differ significantly in glucose tolerance, do not differ significantly in insulin sensitivity, do not differ significantly in glucose-induced insulin secretion, do not differ significantly in beta-cell mass, and do not differ significantly in alpha-cell mass. Compared to non-human animals without the mutation (e.g., wild type non-human animals), non-human animals with a mutated Slc30a8 locus can have a decrease in the ratio of insulin/C-peptide when fed a control chow diet. This may indicate higher insulin clearance.

Compared to non-human animals without the mutation (e.g., wild type non-human animals), non-human animals with a mutated Slc30a8 locus have an increased capacity to secrete insulin. As one example, the non-human animals can have an enhanced capacity for insulin secretion when fed a high-fat diet. Such non-human animals can have increased insulin secretion relative to the non-human animal without the mutation when fed a high-fat diet, wherein the increased insulin secretion is associated with increased beta-cell proliferation or beta-cell mass relative to the non-human animal without the mutation. The increased beta-cell proliferation can be insulin-receptor-dependent (e.g., dependent on an insulin receptor in the beta cells). As another example, the non-human animals can have an enhanced capacity for insulin secretion in response to hyperglycemia, such as severe hyperglycemia. Such non-human animals can have increased insulin secretion in response to hyperglycemia induced by insulin receptor inhibition relative to the non-human animal without the mutation. For example, such non-human animals can have increased insulin secretion relative to the non-human animal without the mutation in response to hyperglycemia induced by insulin receptor inhibition, wherein the increased insulin secretion is not associated with increased beta-cell proliferation or beta-cell mass relative to the non-human animal without the mutation. The increased insulin secretion compared to non-human animals without the mutation can be at least about 5%, 10%, 15%, 20%, 25%, 30%, 35%, 40%, 45%, or 50%. For example, the non-human animals can have increased insulin secretion in response to hyperglycemia induced by insulin receptor inhibition, such as hyperglycemia caused by the insulin receptor antagonist S961. In some non-human animals, this effect is not associated with enhanced beta-cell proliferation or increased beta-cell mass. In some non-human animals, the increase in plasma insulin is not a result of one or more or all of the following: improved proinsulin processing, decreased insulin clearance, increased beta-cell proliferation, increased islet mass, increased beta-cell mass, increased alpha-cell mass, or insulin depletion in beta cells.

Compared to non-human animals without the mutation (e.g., wild type non-human animals), non-human animals with a mutated Slc30a8 locus can have decreased mitochondrial gene expression (e.g., decrease in expression of genes in the mitochondrial oxidative phosphorylation (OXPHOS) system). Examples of such genes are described in more detail in Example 2 and in Tables 7-9. Compared to non-human animals without the mutation (e.g., wild type non-human animals), non-human animals with a mutated Slc30a8 locus can have increased Hvcn1 (hydrogen voltage-gated channel 1, or voltage-gated hydrogen channel 1) expression.

Compared to non-human animals without the mutation (e.g., wild type non-human animals), non-human animals with a mutated Slc30a8 locus can have loss of islet zinc accumulation.

Compared to non-human animals without the mutation (e.g., wild type non-human animals), non-human animals with a mutated Slc30a8 locus can also have one or more or all of the following characteristics when fed a control chow diet: do not differ in body weight, blood glucose, insulin, or C-peptide levels (fed or fasted); have no change in fed proinsulin levels; might have decreased fasted proinsulin levels; have no change in the ratio of proinsulin to insulin (fed or fasted); have an increased fed ratio of C-peptide to insulin; have no change in the fasted ratio of C-peptide to insulin; and have no change in glucose tolerance or glucose-induced insulin secretion.

Compared to non-human animals without the mutation (e.g., wild type non-human animals), non-human animals with a mutated Slc30a8 locus can also have one or more or all of the following characteristics when fed a high-fat diet: have no change in fed or fasted blood glucose; have increased fed circulating insulin levels; have no change in fasted circulating insulin levels; have no change in fed proinsulin levels; have decreased fasted proinsulin levels; have increased fed circulating C-peptide levels; have no change in fasted circulating C-peptide levels; have a decreased ratio of proinsulin to insulin (fed and fasted); have no change in the ratio of C-peptide to insulin; have an increased insulin-positive area in the pancreas; have an increased islet number per pancreas area; have increased beta-cell proliferation; and have an increased number of insulin-producing beta cells. In addition, compared to non-human animals without the mutation, non-human animals with a mutated Slc30a8 locus can have increased fed plasma insulin levels after blockade of the insulin receptor (e.g., using S961).

A mutated Slc30a8 locus disclosed herein can be a Slc30a8 locus encoding a truncated SLC30A8 protein (i.e., protein-truncating variants). The mutation causing the truncation can be, for example, a frameshift mutation, a nonsense mutation, a splice site mutation, or an initiator codon SNV. See, e.g., Flannick et al. (2014) Nat. Genet. 46(4):357-363, herein incorporated by reference in its entirety for all purposes. A nonsense mutation is a mutation in which a sense codon that corresponds to one of the twenty amino acids specified by the genetic code is changed to a chain-terminating codon (i.e., termination codon or stop codon). A frameshift mutation arises when the normal sequence of codons is disrupted by the insertion or deletion of one or more nucleotides, provided that the number of nucleotides added or removed is not a multiple of three. A frameshift mutation is a sequence change between the translation initiation codon (start codon) and termination codon (stop codon) in which, compared to a reference sequence, translation shifts to another frame. For example, the reading frame can be shifted one nucleotide in the 5′ direction (−1 frameshift) or one nucleotide in the 3′ direction (+1 frameshift). A protein encoded by a gene with a frameshift mutation will be identical to the protein encoded by the wild type gene from the N-terminus to the frameshift mutation, but different beyond that point. Such frameshifts can result in a premature termination codon. A splice site mutation can result, for example, in skipping of an exon and potentially a subsequent frameshift that results in a premature termination codon. In one example, a mutated Slc30a8 locus disclosed herein can be a Slc30a8 locus in which the Slc30a8 gene has a premature termination codon (i.e., stop codon). In a specific example, the mutation comprises, consists essentially of, or consists of a nonsense mutation.

The mutation can be anywhere within the Slc30a8 locus. For example, the mutation can be in the first exon, the second exon, the third exon, the fourth exon, the fifth exon, the sixth exon, the seventh exon, or the eighth exon of the Slc30a8 gene. Similarly, the mutation can be in a region of the Slc30a8 gene corresponding to the first, second, third, fourth, fifth, sixth, seventh, or eighth exon of the human SLC30A8 gene when optimally aligned with the human SLC30A8 gene. In a specific example, the mutation is in the third exon of the Slc30a8 gene or in a region of the Slc30a8 gene corresponding to the third exon of the human SLC30A8 gene when optimally aligned with the human SLC30A8 gene. In a more specific example, the mutation is at the 3′ end of the third exon of the Slc30a8 gene or in a region of the Slc30a8 gene corresponding to the 3′ end of third exon of the human SLC30A8 gene when optimally aligned with the human SLC30A8 gene.

In one example, the mutation is at a residue corresponding to residue 412 of the human SLC30A8 coding sequence (CDS) set forth in SEQ ID NO: 21 (i.e., residue c.412, transcript accession number NM_173851) when the coding sequence of the mutated Slc30a8 gene is optimally aligned with SEQ ID NO: 21. Alternatively, the mutation is at a residue within 1, 2, 3, 4, 5, 10, 15, 20, 25, 30, 40, 50, 100, 200, 300, 400, or 500 nucleotides of residue 412 of the human SLC30A8 coding sequence set forth in SEQ ID NO: 21 (i.e., residue c.412, transcript accession number NM_173851) when the coding sequence of the mutated Slc30a8 gene is optimally aligned with SEQ ID NO: 21.

In another example, the mutation is at a residue corresponding to residue 409 of the mouse Slc30a8 coding sequence (CDS) set forth in SEQ ID NO: 20 (i.e., residue c.409, transcript accession number NM_173851.2) when the coding sequence of the mutated Slc30a8 gene is optimally aligned with SEQ ID NO: 20. Alternatively, the mutation is at a residue within 1, 2, 3, 4, 5, 10, 15, 20, 25, 30, 40, 50, 100, 200, 300, 400, or 500 nucleotides of residue 409 of the mouse Slc30a8 coding sequence set forth in SEQ ID NO: 20 (i.e., residue c.409, transcript accession number NM_173851.2) when the coding sequence of the mutated Slc30a8 gene is optimally aligned with SEQ ID NO: 20.

In another example, the mutation results in a stop codon in a codon corresponding to residue 138 in the human SLC30A8 protein set forth in SEQ ID NO: 14 (i.e., amino acid 138 in UniProt accession number Q8IWU4.2) when optimally aligned with SEQ ID NO: 14. Alternatively, the stop codon corresponds to a codon within 1, 2, 3, 4, 5, 10, 15, 20, 25, 30, 40, 50, 60, 70, 80, 90, 100, 110, 120, 130, 140, 150, 160, 170, 180, 190, 200, 210, 220, 230, 240, or 250 amino acids of residue 138 in the human SLC30A8 protein set forth in SEQ ID NO: 14 (i.e., amino acid 138 in UniProt accession number Q8IWU4.2) when optimally aligned with SEQ ID NO: 14.

In another example, the mutation results in a stop codon in a codon corresponding to residue 137 in the mouse SLC30A8 protein set forth in SEQ ID NO: 12 (i.e., amino acid 137 in UniProt accession number Q8BGG0.1) when optimally aligned with SEQ ID NO: 12. Alternatively, the stop codon corresponds to a codon within 1, 2, 3, 4, 5, 10, 15, 20, 25, 30, 40, 50, 60, 70, 80, 90, 100, 110, 120, 130, 140, 150, 160, 170, 180, 190, 200, 210, 220, 230, 240, or 250 amino acids of residue 137 in the mouse SLC30A8 protein set forth in SEQ ID NO: 12 (i.e., amino acid 137 in UniProt accession number Q8BGG0.1) when optimally aligned with SEQ ID NO: 12.

The mutated Slc30a8 locus can be endogenous to the non-human animal. In a specific example, the mutated Slc30a8 locus encodes an mRNA (cDNA) that comprises, consists essentially of, or consists of a sequence that is at least 85%, 90%, 95%, 96%, 97%, 98%, 99%, or 100% identical to SEQ ID NO: 18. The coding sequence of the mutated SLC30A8 locus can comprise, consist essentially of, or consist of a sequence that is at least 85%, 90%, 95%, 96%, 97%, 98%, 99%, or 100% identical to SEQ ID NO: 22 or degenerates thereof encoding the same amino acid sequence. The resulting SLC30A8 protein encoded by the mutated SLC30A8 locus can comprise, consist essentially of, or consist of a sequence that is at least 85%, 90%, 95%, 96%, 97%, 98%, 99%, or 100% identical to SEQ ID NO: 13.

Optionally, a mutated SLC30A8 locus can comprise other exogenous or heterologous elements. Examples of such elements can include selection cassettes, reporter genes, recombinase recognition sites, or other elements. As one example, a mutated SLC30A8 locus can comprise a removable selection cassette (e.g., a self-deleting selection cassette) flanked by recombinase recognition sequences (e.g., loxP sites). Alternatively, the mutated SLC30A8 locus can lack other elements (e.g., can lack a selection cassette and/or can lack a reporter gene). Examples of suitable reporter genes and reporter proteins are disclosed elsewhere herein. Examples of suitable selection markers include neomycin phosphotransferase (neoᵣ), hygromycin B phosphotransferase (hygᵣ), puromycin-N-acetyltransferase (puroᵣ), blasticidin S deaminase (bsrᵣ), xanthine/guanine phosphoribosyl transferase (gpt), and herpes simplex virus thymidine kinase (HSV-k). Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

Other elements such as reporter genes or selection cassettes can be self-deleting cassettes flanked by recombinase recognition sites. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. The polynucleotide encoding the selection marker can be operably linked to a promoter active in a cell being targeted. Examples of promoters are described elsewhere herein. As another specific example, a self-deleting selection cassette can comprise a hygromycin resistance gene coding sequence operably linked to one or more promoters (e.g., both human ubiquitin and EM7 promoters) followed by a polyadenylation signal, followed by a Crei coding sequence operably linked to one or more promoters (e.g., an mPrm1 promoter), followed by another polyadenylation signal, wherein the entire cassette is flanked by loxP sites.

The mutated SLC30A8 locus can also be a conditional allele. For example, the conditional allele can be a multifunctional allele, as described in US 2011/0104799, herein incorporated by reference in its entirety for all purposes. For example, the conditional allele can comprise: (a) an actuating sequence in sense orientation with respect to transcription of a target gene; (b) a drug selection cassette (DSC) in sense or antisense orientation; (c) a nucleotide sequence of interest (NSI) in antisense orientation; and (d) a conditional by inversion module (COIN, which utilizes an exon-splitting intron and an invertible gene-trap-like module) in reverse orientation. See, e.g., US 2011/0104799. The conditional allele can further comprise recombinable units that recombine upon exposure to a first recombinase to form a conditional allele that (i) lacks the actuating sequence and the DSC; and (ii) contains the NSI in sense orientation and the COIN in antisense orientation. See, e.g., US 2011/0104799.

C. Non-Human Animal Genomes, Non-Human Animal Cells, and Non-Human Animals Comprising a Mutated Slc30a8 Locus

Non-human animal genomes, non-human animal cells, and non-human animals comprising a mutated Slc30a8 locus as described elsewhere herein are provided. The genomes, cells, or non-human animals can be male or female. The genomes, cells, or non-human animals can be heterozygous or homozygous for the mutated Slc30a8 locus. A diploid organism has two alleles at each genetic locus. Each pair of alleles represents the genotype of a specific genetic locus. Genotypes are described as homozygous if there are two identical alleles at a particular locus and as heterozygous if the two alleles differ.

The non-human animal genomes or cells provided herein can be, for example, any non-human animal genome or cell comprising a Slc30a8 locus or a genomic locus homologous or orthologous to the human SLC30A8 locus. The genomes can be from or the cells can be eukaryotic cells, which include, for example, animal cells, mammalian cells, non-human mammalian cells, and human cells. The term “animal” includes any member of the animal kingdom, including, for example, mammals, fishes, reptiles, amphibians, birds, and worms. A mammalian cell can be, for example, a non-human mammalian cell, a rodent cell, a rat cell, a mouse cell, or a hamster cell. Other non-human mammals include, for example, non-human primates, monkeys, apes, orangutans, cats, dogs, rabbits, horses, livestock (e.g., bovine species such as cows, steer, and so forth; ovine species such as sheep, goats, and so forth; and porcine species such as pigs and boars). Domesticated animals and agricultural animals are also included. The term “non-human” excludes humans.

The cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells provided herein can also be germ cells (e.g., sperm or oocytes). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be beta cells, pancreatic islet cells, or pancreatic cells.

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, beta cells.

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Specific examples of immortalized cell lines are the EndoC-βH2, 1.1B4, 1.4E7, 1.1E7, RIN, HIT, MIN, INS-1, and βTC cell lines. See, e.g., Scharfmann et al. (2014) J. Clin. Invest. 124(5):2087-2098 and McCluskey et al. (2011) J. Biol. Chem. 286(25):21982-21992, each of which is herein incorporated by reference in its entirety for all purposes. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

Non-human animals comprising a mutated Slc30a8 locus as described herein can be made by the methods described elsewhere herein. The term “animal” includes any member of the animal kingdom, including, for example, mammals, fishes, reptiles, amphibians, birds, and worms. In a specific example, the non-human animal is a non-human mammal. Non-human mammals include, for example, non-human primates, monkeys, apes, orangutans, cats, dogs, horses, rabbits, rodents (e.g., mice, rats, hamsters, and guinea pigs), and livestock (e.g., bovine species such as cows and steer; ovine species such as sheep and goats; and porcine species such as pigs and boars). Domesticated animals and agricultural animals are also included. The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 129S1/SV, 129S1/Svlm), 129S2, 129S4, 129S5, 129S9/SvEvH, 129S6 (129/SvEvTac), 129S7, 129S8, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

### III. Methods of Making Non-Human Animals Comprising a Mutated Slc30a8 Locus

Various methods are provided for making a non-human animal genome, non-human animal cell, or non-human animal comprising a mutated Slc30a8 locus as disclosed elsewhere herein. Any convenient method or protocol for producing a genetically modified organism is suitable for producing such a genetically modified non-human animal. See, e.g., Cho et al. (2009) Current Protocols in Cell Biology 42:19.11:19.11.1-19.11.22 and Gama Sosa et al. (2010) Brain Struct. Funct. 214(2-3):91-109, each of which is herein incorporated by reference in its entirety for all purposes. Such genetically modified non-human animals can be generated, for example, through gene knock-in at a targeted Slc30a8 locus.

For example, the method of producing a non-human animal comprising a mutated Slc30a8 locus can comprise: (1) modifying the genome of a pluripotent cell to comprise the mutated Slc30a8 locus; (2) identifying or selecting the genetically modified pluripotent cell comprising the mutated Slc30a8 locus; (3) introducing the genetically modified pluripotent cell into a non-human animal host embryo; and (4) implanting and gestating the host embryo in a surrogate mother. For example, the method of producing a non-human animal comprising a mutated Slc30a8 locus can comprise: (1) modifying the genome of a pluripotent cell to comprise the mutated Slc30a8 locus; (2) identifying or selecting the genetically modified pluripotent cell comprising the mutated Slc30a8 locus; (3) introducing the genetically modified pluripotent cell into a non-human animal host embryo; and (4) gestating the host embryo in a surrogate mother. Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the mutated Slc30a8 locus.

The methods can further comprise identifying a cell or animal having a modified target genomic locus (i.e., a mutated Slc30a8 locus). Various methods can be used to identify cells and animals having a targeted genetic modification.

The step of modifying the genome can, for example, utilize exogenous repair templates (e.g., targeting vectors) to modify a Slc30a8 locus to comprise an Slc30a8 mutation disclosed herein. As one example, the targeting vector can be for generating a mutated Slc30a8 gene at an endogenous Slc30a8 locus (e.g., endogenous non-human animal Slc30a8 locus), wherein the targeting vector comprises a 5′ homology arm targeting a 5′ target sequence at the endogenous Slc30a8 locus and a 3′ homology arm targeting a 3′ target sequence at the endogenous Slc30a8 locus, wherein the targeting vector comprises a mutation in the Slc30a8 gene, wherein the mutated Slc30a8 gene encodes a truncated SLC30A8 protein. As a specific example, the mutation can result in a stop codon in a codon corresponding to residue 138 in the human SLC30A8 protein.

The exogenous repair templates can be for non-homologous-end-joining-mediated insertion or homologous recombination. Exogenous repair templates can comprise deoxyribonucleic acid (DNA) or ribonucleic acid (RNA), they can be single-stranded or double-stranded, and they can be in linear or circular form. For example, a repair template can be a single-stranded oligodeoxynucleotide (ssODN).

Exogenous repair templates can also comprise nucleic acid inserts including segments of DNA to be integrated in the Slc30a8 locus. Integration of a nucleic acid insert in the Slc30a8 locus can result in addition of a nucleic acid sequence of interest in the Slc30a8 locus, deletion of a nucleic acid sequence of interest in the Slc30a8 locus, or replacement of a nucleic acid sequence of interest in the Slc30a8 locus (i.e., deletion and insertion).

Exogenous repair templates can also comprise a heterologous sequence that is not present at an untargeted endogenous Slc30a8 locus. For example, an exogenous repair template can comprise a selection cassette, such as a selection cassette flanked by recombinase recognition sites.

Some exogenous repair templates comprise homology arms. If the exogenous repair template acid also comprises a nucleic acid insert, the homology arms can flank the nucleic acid insert. For ease of reference, the homology arms are referred to herein as 5′ and 3′ (i.e., upstream and downstream) homology arms. This terminology relates to the relative position of the homology arms to the nucleic acid insert within the exogenous repair template. The 5′ and 3′ homology arms correspond to regions within the Slc30a8 locus, which are referred to herein as “5′ target sequence” and “3′ target sequence,” respectively.

A homology arm and a target sequence “correspond” or are “corresponding” to one another when the two regions share a sufficient level of sequence identity to one another to act as substrates for a homologous recombination reaction. The term “homology” includes DNA sequences that are either identical or share sequence identity to a corresponding sequence. The sequence identity between a given target sequence and the corresponding homology arm found in the exogenous repair template can be any degree of sequence identity that allows for homologous recombination to occur. For example, the amount of sequence identity shared by the homology arm of the exogenous repair template (or a fragment thereof) and the target sequence (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target sequence can be of any length that is sufficient to promote homologous recombination. In some targeting vectors, the intended mutation in the Slc30a8 locus is included in one of the homology arms. In other targeting vectors, the intended mutation in the Slc30a8 locus is included in an insert nucleic acid flanked by the homology arms.

In cells other than one-cell stage embryos, the exogenous repair template can be a “large targeting vector” or “LTVEC,” which includes targeting vectors that comprise homology arms that correspond to and are derived from nucleic acid sequences larger than those typically used by other approaches intended to perform homologous recombination in cells. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb.

The screening step can comprise, for example, a quantitative assay for assessing modification of allele (MOA) of a parental chromosome. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Modification-of-allele (MOA) assays including loss-of-allele (LOA) and gain-of-allele (GOA) assays are described, e.g., in US 2014/0178879 and Frendewey et al. (2010) Methods Enzymol. 476:295-307, each of which is herein incorporated by reference in its entirety for all purposes. Retention assays as described in US 2016/0145646 and WO 2016/081923, each of which is herein incorporated by reference in its entirety for all purposes, can also be used.

Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

An example of a suitable pluripotent cell is an embryonic stem (ES) cell (e.g., a mouse ES cell or a rat ES cell). The modified pluripotent cell can be generated, for example, through recombination by (a) introducing into the cell one or more exogenous donor nucleic acids (e.g., targeting vectors) comprising an optional insert nucleic acid flanked, for example, by 5′ and 3′ homology arms corresponding to 5′ and 3′ target sites, wherein the insert nucleic acid or one of the homology arms comprises a mutated Slc30a8 locus or the mutation to be made at the Slc30a8 locus; and (b) identifying at least one cell comprising in its genome the insert nucleic acid integrated at the endogenous Slc30a8 locus. The modified pluripotent cell can be generated, for example, through recombination by (a) introducing into the cell one or more targeting vectors comprising an insert nucleic acid flanked by 5′ and 3′ homology arms corresponding to 5′ and 3′ target sites, wherein the insert nucleic acid comprises a mutated Slc30a8 locus or the mutation to be made at the Slc30a8 locus; and (b) identifying at least one cell comprising in its genome the insert nucleic acid integrated at the endogenous Slc30a8 locus. Any targeting vector can be used. In one example, a large targeting vector (LTVEC) is used. The LTVEC can be, for example, at least 10 kb in length or can have 5′ and 3′ homology arms, the sum total of which is at least 10 kb in length. As another example, the targeting vector can be a single-stranded oligodeoxynucleotide (ssODN). The ssODN can be, for example, between about 80 to about 200 nucleotides in length.

Alternatively, the modified pluripotent cell can be generated by (a) introducing into the cell: (i) a nuclease agent, wherein the nuclease agent induces a nick or double-strand break at a recognition site within the endogenous Slc30a8 locus; and (ii) one or more exogenous donor nucleic acids (e.g., targeting vectors) optionally comprising an insert nucleic acid flanked by, for example, 5′ and 3′ homology arms corresponding to 5′ and 3′ target sites located in sufficient proximity to the recognition site, wherein the insert nucleic acid or one of the homology arms comprises the mutated Slc30a8 locus or the mutation to be made at the Slc30a8 locus; and (c) identifying at least one cell comprising a modification (e.g., integration of the insert nucleic acid) at the endogenous Slc30a8 locus. Alternatively, the modified pluripotent cell can be generated by (a) introducing into the cell: (i) a nuclease agent, wherein the nuclease agent induces a nick or double-strand break at a recognition site within the endogenous Slc30a8 locus; and (ii) one or more targeting vectors comprising an insert nucleic acid flanked by 5′ and 3′ homology arms corresponding to 5′ and 3′ target sites located in sufficient proximity to the recognition site, wherein the insert nucleic acid comprises the mutated Slc30a8 locus or the mutation to be made at the Slc30a8 locus; and (c) identifying at least one cell comprising a modification (e.g., integration of the insert nucleic acid) at the endogenous Slc30a8 locus. Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes. Examples of suitable methods for making targeted modifications to cells are described, e.g., in US 2016/0145646 and WO 2016/081923, each of which is herein incorporated by reference in its entirety for all purposes.

The donor cell can be introduced into a host embryo at any stage, such as the blastocyst stage or the pre-morula stage (i.e., the 4-cell stage or the 8-cell stage). Progeny that are capable of transmitting the genetic modification though the germline are generated. See, e.g., U.S. Pat. No. 7,294,754, herein incorporated by reference in its entirety for all purposes.

Alternatively, the method of producing the non-human animals described elsewhere herein can comprise: (1) modifying the genome of a one-cell stage embryo to comprise the mutated Slc30a8 locus using the methods described above for modifying pluripotent cells; (2) selecting the genetically modified embryo; and (3) implanting and gestating the genetically modified embryo into a surrogate mother. Alternatively, the method of producing the non-human animals described elsewhere herein can comprise: (1) modifying the genome of a one-cell stage embryo to comprise the mutated Slc30a8 locus using the methods described above for modifying pluripotent cells; (2) selecting the genetically modified embryo; and (3) gestating the genetically modified embryo in a surrogate mother. Progeny that are capable of transmitting the genetic modification though the germline are generated.

Nuclear transfer techniques can also be used to generate the non-human animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The various methods provided herein allow for the generation of a genetically modified non-human F0 animal wherein the cells of the genetically modified F0 animal comprise the mutated Slc30a8 locus. Depending on the method used to generate the F0 animal, the number of cells within the F0 animal that have the mutated Slc30a8 locus will vary. The introduction of the donor ES cells into a pre-morula stage embryo from a corresponding organism (e.g., an 8-cell stage mouse embryo) via for example, the VELOCIMOUSE® method allows for a greater percentage of the cell population of the F0 animal to comprise cells having the nucleotide sequence of interest comprising the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal can comprise a cell population having the targeted modification.

The cells of the genetically modified F0 animal can be heterozygous for the mutated Slc30a8 locus or can be homozygous for the mutated Slc30a8 locus.

### IV. Methods of Screening Compounds

The non-human animals having the mutated Slc30a8 loci described herein can be used for screening compounds for activity potentially useful in inhibiting, ameliorating, or reducing type 2 diabetes or ameliorating type-2-diabetes-like symptoms or screening compounds for activity potentially harmful in promoting or exacerbating type 2 diabetes or type-2-diabetes-like symptoms. Wild type non-human animals or non-human animals that do not have the mutated Slc30a8 loci described herein can also be used for screening compounds for activity potentially useful in inhibiting, ameliorating, or reducing type 2 diabetes or ameliorating type-2-diabetes-like symptoms or screening compounds for activity potentially harmful in promoting or exacerbating type 2 diabetes or type-2-diabetes-like symptoms by monitoring phenotypic aspects associated with the protective mutated Slc30a8 loci described herein. Compounds having activity inhibiting, ameliorating, or reducing type 2 diabetes or ameliorating type-2-diabetes-like symptoms are potentially useful as therapeutics or prophylactics against type 2 diabetes. Compounds having activity promoting or exacerbating type 2 diabetes or type-2-diabetes-like symptoms are identified as toxic and should be avoided as therapeutics or in other circumstances in which they may come into contact with humans (e.g., in foods, agriculture, construction, or water supply).

Examples of compounds that can be screened include antibodies, antigen-binding proteins, site-specific DNA binding proteins (e.g., CRISPR-Cas complexes), polypeptides, beta-turn mimetics, polysaccharides, phospholipids, hormones, prostaglandins, steroids, aromatic compounds, heterocyclic compounds, benzodiazepines, oligomeric N-substituted glycines, and oligocarbamates. Large combinatorial libraries of the compounds can be constructed by the encoded synthetic libraries (ESL) method described in WO 1995/012608, WO 1993/006121, WO 1994/008051, WO 1995/035503, and WO 1995/030642, each of which is herein incorporated by reference in its entirety for all purposes. Peptide libraries can also be generated by phage display methods. See, e.g., U.S. Pat. No. 5,432,018, herein incorporated by reference in its entirety for all purposes. Use of libraries of guide RNAs for targeting CRISPR-Cas systems to different genes are disclosed, e.g., in WO 2014/204727, WO 2014/093701, WO 2015/065964, and WO 2016/011080, each of which is herein incorporated by reference in its entirety for all purposes.

Animal-based assays generally involve administering a compound to the non-human animal comprising the mutated Slc30a8 locus and assessing signs or symptoms closely resembling those of type 2 diabetes, signs or symptoms related to type 2 diabetes, or phenotypic aspects related to glucose, insulin, glucose metabolism, or type 2 diabetes in humans for change in response. The change can be assessed before and after contacting the non-human animal with the compound or by performing a control experiment performed with a control animal having the same Slc30a8 mutation (e.g., wild type cohort sibling) without the compound.

Suitable phenotypic aspects that can be monitored include, for example, capacity to secrete insulin. For example, the capacity to secrete insulin when fed a high-fat diet can be monitored. Alternatively, the capacity to secrete insulin in response to hyperglycemia, such as severe hyperglycemia, can be monitored. For example, the insulin secretion can be in response to hyperglycemia induced by insulin receptor inhibition, such as hyperglycemia caused by the insulin receptor antagonist S961.

Other suitable phenotypic aspects that can be monitored include mitochondrial gene expression (e.g., expression of genes in the mitochondrial oxidative phosphorylation (OXPHOS) system) or expression of Hvcn1. Examples of such mitochondrial genes are described in more detail in Example 2 and in Tables 6-8. Other suitable phenotypic aspects that can be monitored include ratio of insulin/C-peptide when fed a control chow diet or insulin clearance. Other suitable phenotypic aspects that can be monitored include islet zinc accumulation.

Other suitable phenotypic aspects that can be monitored include, for example, (1) glucose-induced insulin secretion when fed a high-fat diet (e.g., circulating insulin levels after 20 weeks being fed on a high-fat diet); (2) pancreatic beta-cell proliferation levels when fed a high-fat diet (e.g., after 20 weeks being fed on a high-fat diet); (3) number of pancreatic beta cells when fed a high-fat diet (e.g., after 20 weeks being fed on a high-fat diet); and (4) fed plasma insulin levels after blockade of the insulin receptor (e.g., with using S961). Alternatively or additionally, suitable phenotypic aspects that can be monitored include, for example, proinsulin-to-insulin ratio when fed a high-fat diet or insulin processing and health of pancreatic beta cells.

For example, activity for exacerbating type 2 diabetes can be identified by one or both of: (1) decreased capacity to secrete insulin (e.g., when fed a high-fat diet or in response to hyperglycemia) or (2) reduced insulin clearance (e.g., increased ratio of insulin/C-peptide when fed a control chow diet). Activity for exacerbating type 2 diabetes can also be identified by one or both of (1) increased mitochondrial gene expression (e.g., expression of genes in the mitochondrial oxidative phosphorylation (OXPHOS) system) or (2) decreased expression of Hvcn1.

Activity for exacerbating type 2 diabetes can also be identified by one or more or all of: (1) decreased glucose-induced insulin secretion when fed a high-fat diet (e.g., decreased circulating insulin levels after 20 weeks being fed on a high-fat diet); (2) decreased pancreatic beta-cell proliferation when fed a high-fat diet (e.g., after 20 weeks being fed on a high-fat diet); (3) decreased number of pancreatic beta cells when fed a high-fat diet (e.g., after 20 weeks being fed on a high-fat diet); and (4) decreased fed plasma insulin levels after blockade of the insulin receptor. Alternatively or additionally, activity for exacerbating type 2 diabetes can be identified by one or more or all of increased ratio of proinsulin-to-insulin and worsened insulin processing and health of pancreatic beta cells (e.g., as evidenced by increased ratio of proinsulin-to-insulin when fed a high-fat diet).

Alternatively, activity for ameliorating type 2 diabetes can be identified by one or both of: (1) increased capacity to secrete insulin (e.g., when fed a high-fat diet or in response to hyperglycemia) or (2) increased insulin clearance (e.g., increased ratio of insulin/C-peptide when fed a control chow diet). Activity for exacerbating type 2 diabetes can also be identified by one or both of: (1) decreased mitochondrial gene expression (e.g., expression of genes in the mitochondrial oxidative phosphorylation (OXPHOS) system) or (2) increased expression of Hvcn1.

Activity for ameliorating type 2 diabetes can also be identified by one or more or all of (1) increased glucose-induced insulin secretion when fed a high-fat diet (e.g., increased circulating insulin levels after 20 weeks being fed on a high-fat diet); (2) increased pancreatic beta-cell proliferation when fed a high-fat diet (e.g., after 20 weeks being fed on a high-fat diet); (3) increased number of pancreatic beta cells when fed a high-fat diet (e.g., after 20 weeks being fed on a high-fat diet); and (4) increased fed plasma insulin levels after blockade of the insulin receptor. Alternatively or additionally, activity for ameliorating type 2 diabetes can be identified by one or more or all of decreased ratio of proinsulin-to-insulin and improved insulin processing and health of pancreatic beta cells (e.g., as evidenced by decreased ratio of proinsulin-to-insulin when fed a high-fat diet).

Such phenotypic aspects can be assayed as described in the examples provided herein. The decrease or increase can be statistically significant. For example, the decrease or increase can be by at least about 1%, at least about 2%, at least about 3%, at least about 4%, at least about 5%, at least about 10%, at least about 15%, at least about 20%, at least about 30%, at least about 40%, at least about 50%, at least about 60%, at least about 70%, at least about 80%, at least about 90%, or 100%.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2
Description of Sequences.

| SEQ ID NO | Type | Description |
|---|---|---|
| 1 | DNA | 8084 AS Fwd Primer |
| 2 | DNA | 8084 AS WT Probe |
| 3 | DNA | 8084 AS Rev Primer |
| 4 | DNA | 8084 AS Mut Probe |
| 5 | DNA | 8084 TD Fwd Primer |
| 6 | DNA | 8084 TD Probe |
| 7 | DNA | 8084 TD Rev Primer |
| 8 | DNA | Slc30a8 intron 2/Slc30a8 exon 3 pArg137*/ |
| | | Slc30a8 intron 3 |
| 9 | DNA | Slc30a8 intron 3/XhoI/(loxP) self-deleting |
| | | neomycin selection cassette |
| 10 | DNA | Self-deleting neomycin selection cassette (loxP)/ |
| | | ICEUI/NheI/Slc30a8 intron 3 |
| 11 | DNA | Slc30a8 intron 3/XhoI/loxP/ICEUI/NheI/Slc30a8 |
| | | intron 3 |
| 12 | Protein | Mouse SLC30A8 WT (UniProt Q8BGG0.1) |
| 13 | Protein | Mouse SLC30A8 p.Arg137X |
| 14 | Protein | Human SLC30A8 WT (UniProt Q8IWU4.2) |
| 15 | Protein | Human SLC30A8 p.Arg138X |
| 16 | DNA | Mouse Slc30a8 cDNA WT (NM_172816.3) |
| 17 | DNA | Human SLC30A8 cDNA WT (NM_173851.2) |
| 18 | DNA | Mouse Slc30a8 cDNA c.409C > T |
| 19 | DNA | Human SLC30A8 cDNA c.412C > T |
| 20 | DNA | Mouse Slc30a8 CDS WT |
| 21 | DNA | Human SLC30A8 CDS WT |
| 22 | DNA | Mouse Slc30a8 CDS c.409C > T |
| 23 | DNA | Human SLC30A8 CDS c.412C > T |
| 24 | DNA | Deleted Sequence in Mouse Slc30a8 in |
| | | p.Arg137X Allele (MAID 8084) |
| 25 | DNA | Probe |
| 26 | DNA | F Primer |
| 27 | DNA | R Primer |

### EXAMPLES

### Example 1. Generation of Mice Comprising a Mutated Slc30a8 Locus

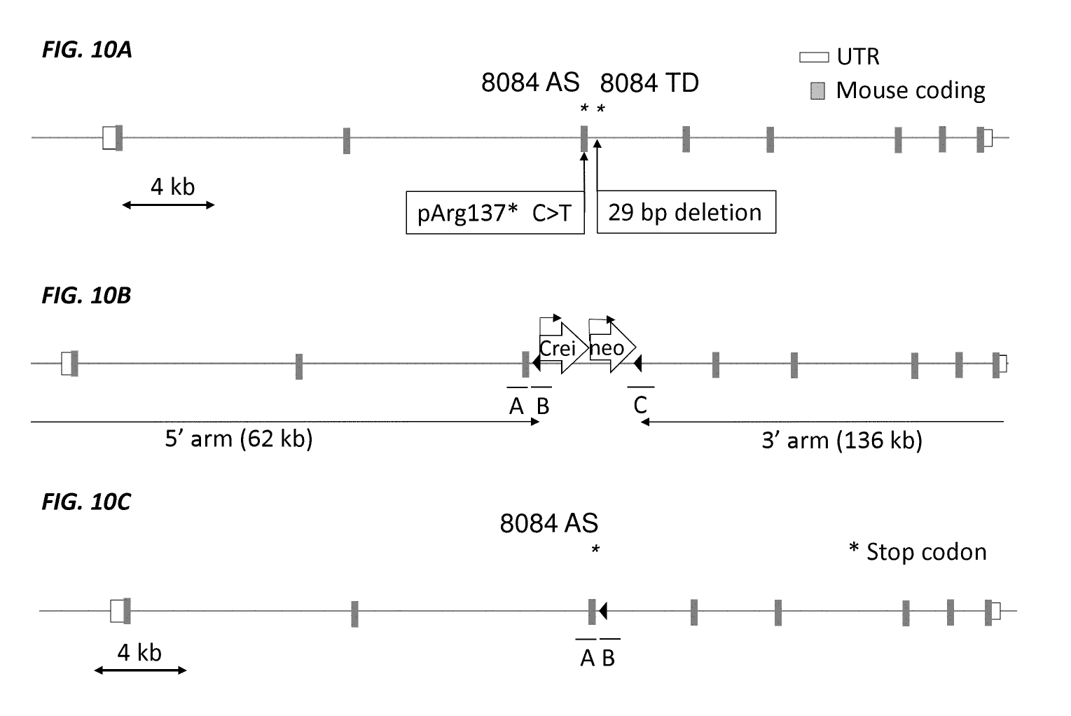

To generate a mouse model carrying an allele corresponding to putative human SLC30A8 LOF allele (c.412C>T (transcript accession number NM_173851.2), p.Arg138X), a mutated mouse Slc30a8 allele was generated (c.409C>T (transcript accession number NM_172816.3), p.Arg137X). Information on the mouse Slc30a8 and human SLC30A8 genes is provided in Table 3. An alignment of the human SLC30A8 and the mouse SLC30A8 proteins is shown in FIG. 11. The mutated allele has a replacement of dC for dT (c.409C>T) at the mouse Slc30a8 codon encoding R137 in exon 3, changing the codon from CGA to TGA (p.Arg137X), making a stop codon expected to produce a truncated protein. The mutated allele also has a self-deleting neomycin selection cassette flanked by loxP sites (loxP-mPrm1-Crei-hUb1-em7-Neo-pA-loxP cassette (4,810 bp)) inserted at intron 3, deleting 29 bp of endogenous intron 3 sequence. The endogenous Slc30a8 locus is shown in FIG. 10A, and the targeted Slc30a8 locus is shown in FIG. 10B. The expected sequence for the region including the boundaries of Slc30a8 intron 2/Slc30a8 exon 3 pArg137*/Slc30a8 intron 3 (denoted by horizontal line “A” in FIG. 10B) is set forth in SEQ ID NO: 8. The expected sequence for the region including the boundaries of Slc30a8 intron 3/XhoI/(loxP) self-deleting cassette (denoted by horizontal line “B” in FIG. 10B) is set forth in SEQ ID NO: 9. The expected sequence for the region including the boundaries of self-deleting cassette (loxP)/ICEUI/NheI/Slc30a8 intron 3 (denoted by horizontal line “C” in FIG. 10B) is set forth in SEQ ID NO: 10.

TABLE 3
Mouse Slc30a8 and Human SLC30A8.

| | Mouse | Human |
|---|---|---|
| Official Symbol | Slc30a8 | SLC30A8 |
| NCBI GeneID | 239436 | 169026 |
| Primary Source | MGI: 2442682 | HGNC: 20303 |
| RefSeq mRNA | NM_172816.3 | NM_173851.2 |
| ID | | |
| UniProt ID | Q8BGG0 | Q8IWU4 |
| Genomic | GRCm38.p4 | GRCh38.p7 |
| Assembly | | |
| Location | Chr 15: | Chr 8: |
| | 52295553-52335733 (+) | 116950273-117176714 (+) |

The targeted Slc30a8 locus after deletion of the self-deleting loxP-mPrm1-Crei-hUb1-em7-Neo-pA-loxP cassette is shown in FIG. 10C. After cassette deletion, loxP and cloning sites (77 bp) remain in Slc30a8 intron 3. The expected sequence for the region including the boundaries of Slc30a8 intron 2/Slc30a8 exon 3 pArg137*/Slc30a8 intron 3 (denoted by horizontal line “A” in FIG. 10C) is set forth in SEQ ID NO: 8. The expected sequence for the region including the boundaries of Slc30a8 intron 3/XhoI/loxP/ICEUI/NheI/Slc30a8 intron 3 (denoted by horizontal line “B” in FIG. 10C) is set forth in SEQ ID NO: 11.

A large targeting vector was generated comprising a 5′ homology arm including 62 kb of sequence (but with the point mutation in the mouse Slc30a8 codon encoding R137) upstream from the region in Slc30a8 targeted for deletion and 136 kb of the sequence downstream of the region in Slc30a8 targeted for deletion. The large targeting vector was designed to introduce a point mutation in the mouse Slc30a8 codon in exon 3 encoding R137 and to replace the 29 bp region in intron 3 with the 4,810 bp self-deleting neomycin selection cassette. See FIG. 10B. The self-deleting neomycin selection cassette included the following components from 5′ to 3′: loxP site, mouse protamine (Prm1) promoter, Crei (Cre coding sequence optimized to include intron), human ubiquitin promoter, EM7 promoter, neomycin phosphotransferase (neoᵣ) coding sequence, polyA, and loxP site. To generate the targeted allele, CRISPR/Cas9 components were introduced into F1H4 mouse embryonic stem cells together with the large targeting vector. Loss-of-allele assays and allele-specific TAQMAN® assays using the primers and probes set forth in Table 4 were performed to confirm correct modification of the Slc30a8 allele. The loss-of-allele assays are denoted as “8084 TD” (TAQMAN® Downstream Assay) and the allele-specific assays are denoted as “8084 AS” (Allele-Specific Assay) in FIG. 10A. Such assays are described, for example, in US 2014/0178879; US 2016/0145646; WO 2016/081923; and Frendewey et al. (2010) Methods Enzymol. 476:295-307, each of which is herein incorporated by reference in its entirety for all purposes.

TABLE 4
Assays for Detection of Targeted Slc30a8 Locus.

| Assay | Primer or Probe | Sequence |
|---|---|---|
| 8084 AS WT | Fwd | CGAGGCCCCCTTCCAA |
| | | (SEQ ID NO: 1) |
| | Probe (FAM-MGB) | CATTTGGGTGGTATCGA |
| | | (SEQ ID NO: 2) |
| | Rev | TGCGCAATCAGAGAATGTTACC |
| | | (SEQ ID NO: 3) |
| 8084 AS Mut | Fwd | CGAGGCCCCCTTCCAA |
| | | (SEQ ID NO: 1) |
| | Probe (VIC-MGB) | CATTTGGGTGGTATTGA |
| | | (SEQ ID NO: 4) |
| | Rev | TGCGCAATCAGAGAATGTTACC |
| | | (SEQ ID NO: 3) |
| 8084 TD | Fwd | TGTGGGAATGTGAGCTCTCAAC |
| | | (SEQ ID NO: 5) |
| | Probe (Cal O-BHQ1) | CAGCTGACAGCAAGATTAATGGAAGTACC |
| | | (SEQ ID NO: 6) |
| | Rev | GACCCTGGAGACAGAAATGTC |
| | | (SEQ ID NO: 7) |

F0 mice were then generated from targeted ES cell clones using the VELOCIMOUSE® method. See, e.g., U.S. Pat. Nos. 7,576,259; 7,659,442; 7,294,754; US 2008/007800; and Poueymirou et al. (2007) Nature Biotech. 25(1):91-99, each of which is herein incorporated by reference in its entirety for all purposes.

The sequence of the expected SLC30A8 protein expressed from the wild type mouse locus is set forth in SEQ ID NO: 12. The sequence of the expected truncated SLC30A8 protein expressed from the targeted mouse locus (SLC30A8 Arg137*) is set forth in SEQ ID NO: 13. An alignment of the two sequences is shown in FIG. 11.

### Example 2. Characterization of SLC30A8 R138X Mice

### Summary

SLC30A8 encodes a zinc transporter that is primarily expressed in the pancreatic islets of Langerhans. In beta cells, it transports zinc into insulin-containing secretory granules. Loss-of-function (LOF) mutations in SLC30A8 protect against type 2 diabetes in humans. Several Slc30a8 knockout mouse lines have been characterized but do not fully recapitulate the human phenotype. Indeed, Slc30a8 deficient mice have reduced plasma insulin levels and impaired glucose tolerance, a phenotype that we replicate here in control Slc30a8-deficient mice. We generated a knock-in mouse model carrying a mutation in the mouse Slc30a8 locus (c.409C>T (transcript accession number NM_172816.3), p.Arg137X) corresponding to the human SLC30A8 putative LOF mutation, R138X. Although the mutation in the mouse Slc30a8 gene is in the codon encoding amino acid residue 137 of the mouse SLC30A8 protein, these mice are referred to herein as “R138X mice” in reference to the corresponding human mutation. Interestingly, the R138X mice had increased glucose-induced insulin secretion when fed a high-fat diet. This was associated with increased beta-cell proliferation and expansion of the beta-cell area. Importantly, heterozygous R138X mice showed a similar increase in glucose-induced insulin secretion. In contrast, chow-fed R138X mice had normal body weight, glucose tolerance, and pancreatic beta-cell mass. Interestingly, in hyperglycemic conditions induced by the insulin receptor antagonist S961, the R138X mice showed about 50% increase in insulin secretion. This effect was not associated with enhanced beta-cell proliferation or beta-cell mass. These data suggest that the SLC30A8 R138X mutation in humans protects against type 2 diabetes in part by increasing the capacity of beta cells to secrete insulin. This may in part be mediated by an increased number of pancreatic beta cells.

### Introduction

SLC30A8 is a zinc transporter (ZnT8) located in the islet cells of the endocrine pancreas. In the beta cells, SLC30A8 transports zinc into the insulin-containing granules. Two zinc ions bind with and stabilize a hexameric form of insulin triggering insulin crystallization. See, e.g., Dodson and Steiner (1998) Curr. Opin. Struct. Biol. 8(2):189-194, herein incorporated by reference in its entirety for all purposes. While loss of islet SLC30A8 substantially decreases islet zinc content and insulin crystallization, its effects on proinsulin processing, secretion and regulation of glucose metabolism are still unclear. See, e.g., Lemaire et al. (2009) Proc. Natl. Acad. Sci. U.S.A. 106(35):14872-14877 and Nicolson et al. (2009) Diabetes 58(9):2070-2083, each of which is herein incorporated by reference in its entirety for all purposes. Several groups have generated Slc30a8 knockout (KO) mouse models either globally or restricted to the pancreatic beta cells. The observed metabolic phenotype of these models is weak, and, although there are differences between studies, most of the models show either no change or a mild impairment of glucose tolerance associated with reduced or unchanged plasma insulin levels. See, e.g., Rutter et al. (2016) Proc. Nutr. Soc. 75(1):61-72, herein incorporated by reference in its entirety for all purposes. Most importantly, none of the studies have reported beneficial effects associated with Slc30a8 deficiency. Therefore, it came as a surprise when Flannick and colleagues reported that haploinsufficiency of SLC30A8 is protective against the development of type 2 diabetes (T2D) in humans. See, e.g., Flannick et al. (2014) Nat. Genet. 46(4):357-363, herein incorporated by reference in its entirety for all purposes. Their study identified 12 putative loss-of-function (LOF) variants that collectively decreased the risk for T2D by 65%. One of the most abundant variants, p.Arg138* (referred to as R138X herein), causing a premature stop codon, was individually associated with protection from T2D and was reported to encode an unstable ZnT8 protein. See, e.g., Flannick et al. (2014) Nat. Genet. 46(4):357-363, herein incorporated by reference in its entirety for all purposes. Thus, mouse and human data seemingly contradict each other, and it is currently not clear how the rare putative LOF variants reduce the risk of T2D.

To investigate this discrepancy between the effect of human SLC30A8 putative LOF variants and the Slc30a8 KO mouse models as well as gain insight as to why SLC30A8 putative LOF variants are protective in humans, we generated and phenotyped a new Slc30a8 mouse model carrying the R138X mutation as a representative human putative LOF variant. Interestingly, we found that the beta cells in the R138X mice have an extraordinary capacity to secrete insulin in response to severe hyperglycemia.

### Results

R138X Islets Appear to Show Loss of SLC30A8 Function Despite Partial Protection of the R138X Transcript from Nonsense-Mediated Decay.

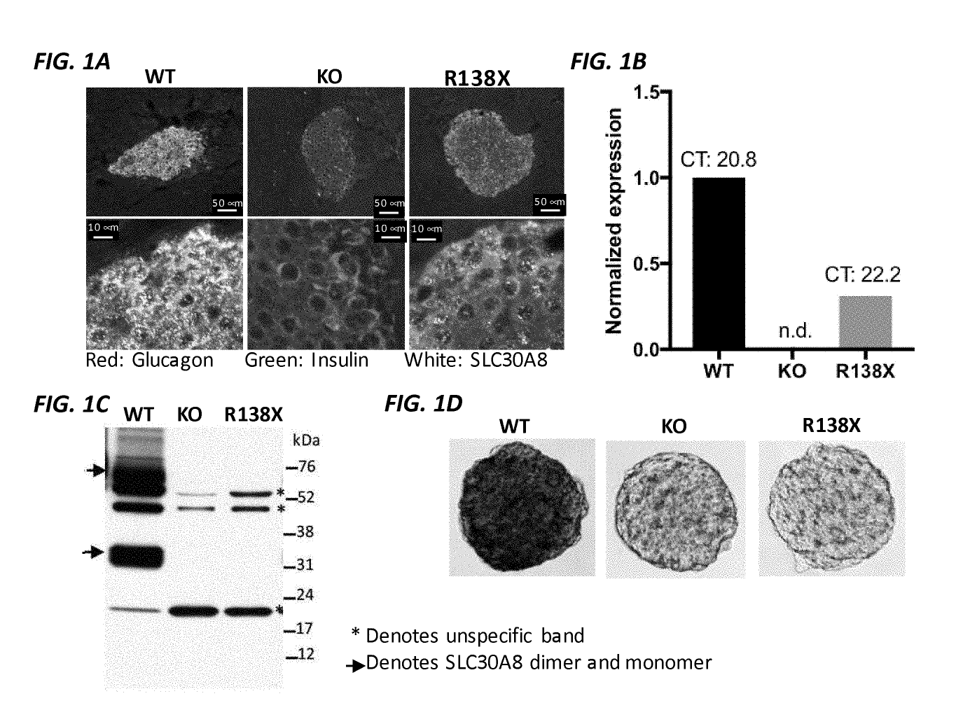

To investigate how human SLC30A8 putative LOF variants influence the risk of T2D, we altered the codon of Slc30a8 in the mouse genome encoding amino acid residue 137 (codon 137, equivalent to codon 138 in human) from CGA to TGA, changing the arginine into a stop codon (R138X) as detailed in Example 1. The R138X mice were born with the expected Mendelian ratio and no obvious growth abnormalities. RNA in situ hybridization showed strong Slc30a8 mRNA staining in pancreatic islets from wild type (WT) mice but no staining in islets from homozygous Slc30a8 KO mice, validating the specificity of the probe (FIG. 1A). In contrast to islets from Slc30a8 KO mice, islets from R138X mice showed a substantial amount of Slc30a8 RNA staining (FIG. 1A). Quantification of Slc30a8 mRNA using real-time PCR revealed a 70% reduction in R138X islets, although the absolute Slc30a8 transcript level in the R138X islets (CT value: 22.2) was still high and comparable with the expression level of the housekeeping gene Gapdh (CT value: 22.1) (FIG. 1B). The presence of this high level of Slc30a8 RNA in the R138X mice suggests that the R138X transcript cannot effectively be degraded by nonsense-mediated mRNA decay, a process that degrades mRNAs carrying premature translation termination codons. See, e.g., Holbrook et al. (2004) Nat. Genet. 36(8):801-808, herein incorporated by reference in its entirety for all purposes.

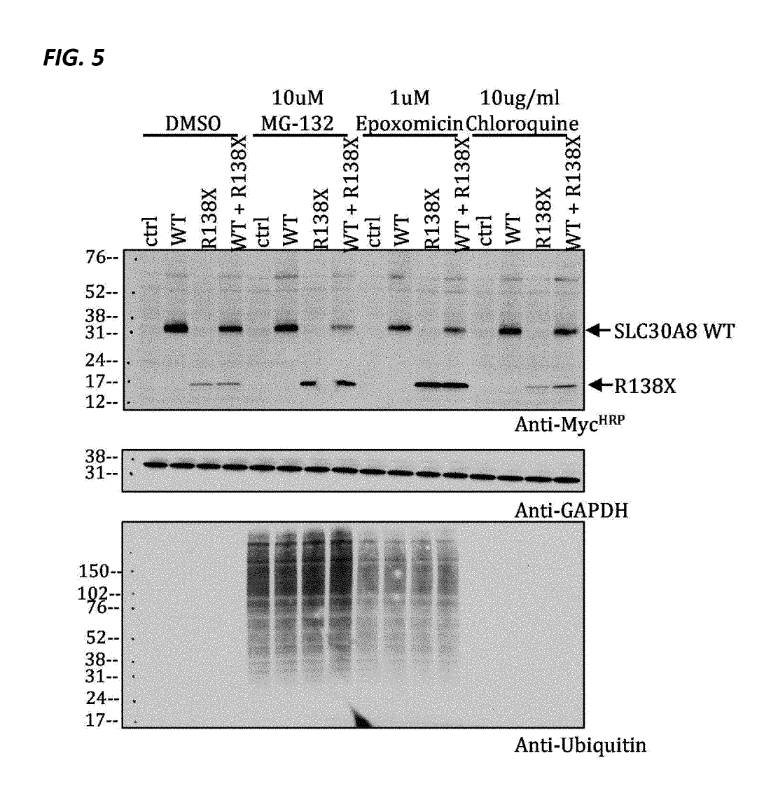

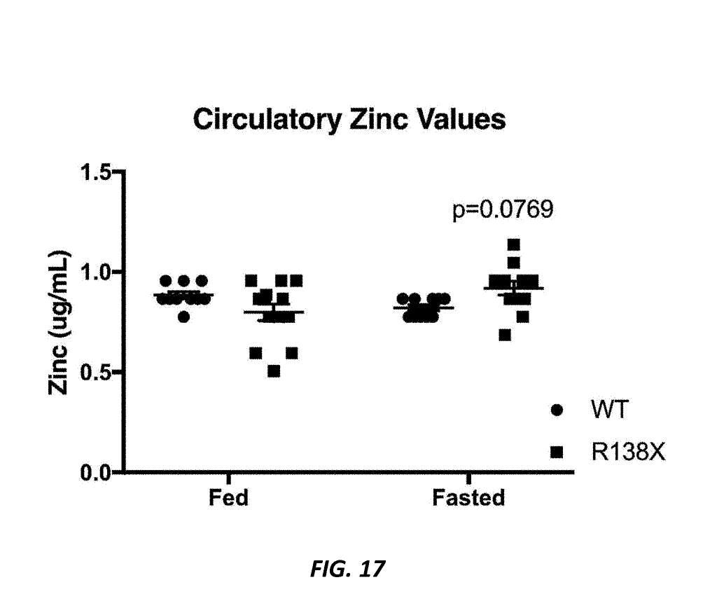

To determine whether the detected RNA in the R138X mice is translated into a truncated protein, we performed Western (immuno-) blotting for SLC30A8 protein using an antibody raised against an N-terminal peptide of the mouse protein. We were not able to identify a truncated protein at the expected molecular weight (˜16 kDa for the monomer) in lysates from isolated islets under the current conditions (FIG. 1C), despite being able to detect the R138X protein and stabilize it by proteasomal inhibition when overexpressed in HEK293 cells (FIG. 5). Similarly, mass spectrometry analysis of whole cell lysates or immunoprecipitates from WT and R138X islets did not identify SLC30A8 peptides in R138X islets (data not shown). In addition, zinc staining using dithizone revealed that islets from KO and R138X mice were zinc depleted demonstrating the absence of a functional SLC30A8 protein in both mouse lines (FIG. 1D). However, no significant change was observed in circulating zinc levels (FIG. 17). In summary, introduction of the human R138X putative LOF variant into the mouse Slc30a8 gene appears to result in loss of SLC30A8 function.

R138X Mice on Chow Diet have a Normal Metabolic Phenotype.

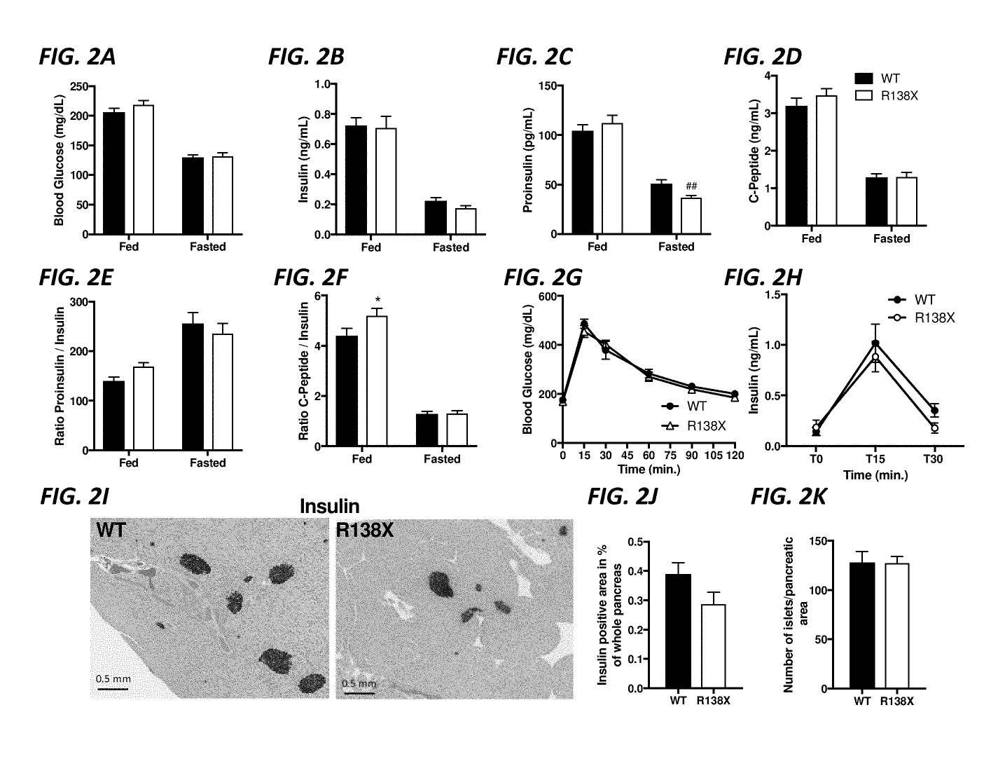

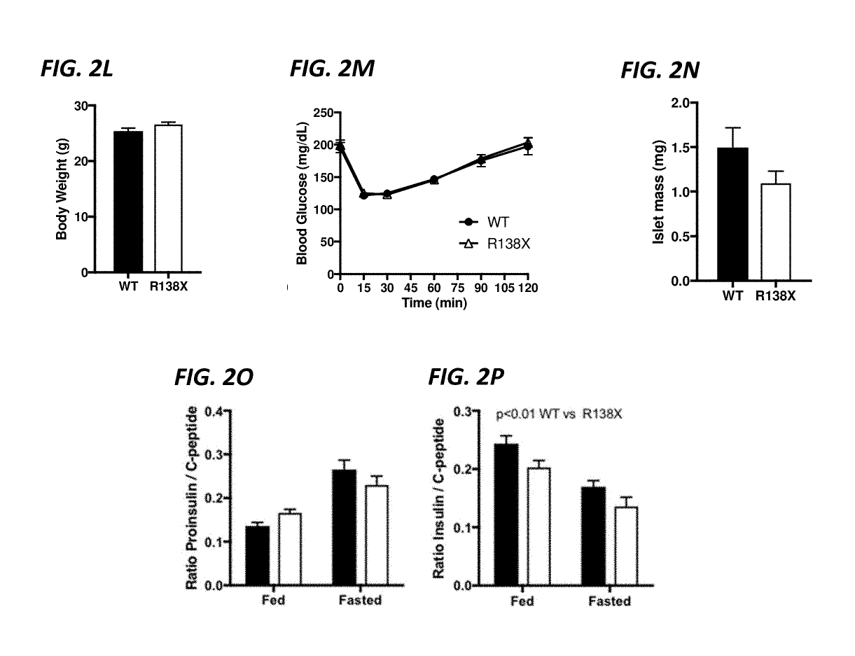

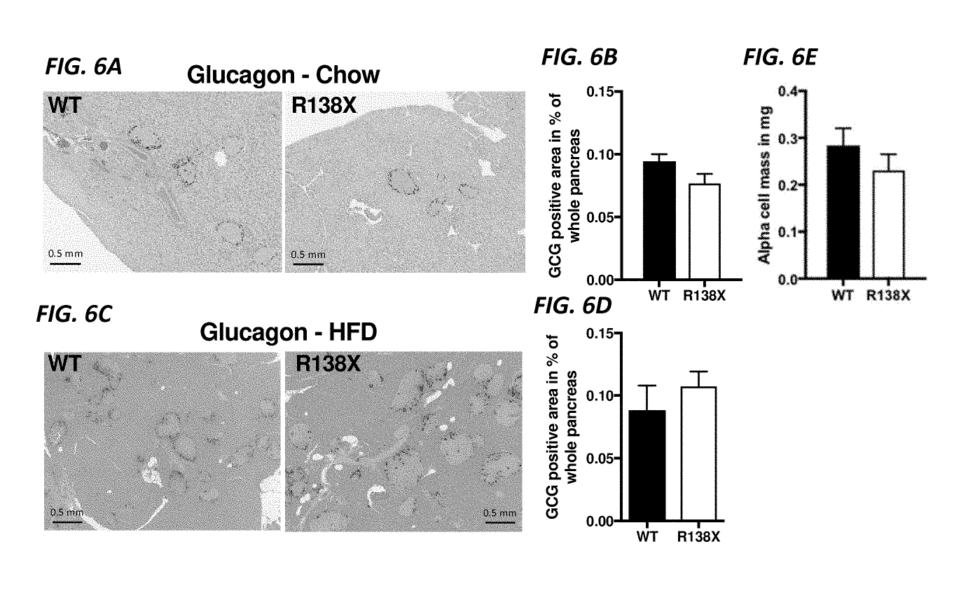

We first investigated whether the introduction of the R138X stop codon altered metabolic parameters in chow-fed mice. WT and R138X mice did not differ in their body weight and circulating blood glucose and insulin levels (FIGS. 2A-2C, 2L; Table 5). Because zinc has been implicated in insulin processing and clearance, we also measured circulating proinsulin and C-peptide levels and calculated the ratio of these hormones to insulin (FIGS. 2C-2F). While there was no difference in circulating C-peptide, fasted proinsulin levels were slightly reduced in the R138X mice (FIG. 2C). The ratio of proinsulin to insulin did not differ between WT and R138X mice, but the ratio of C-peptide to insulin in the fed state was slightly elevated suggesting reduced insulin clearance (FIGS. 2E-2F). Circulating proinsulin and C-peptide levels were similar between WT and R138X mice (FIGS. 2C-2D). While the ratio of proinsulin/C-peptide did not change, we did observe a mild decrease in the ratio of insulin/C-peptide, suggesting higher insulin clearance in R138X mice similar to what has been observed in Slc30a8 KO mice (FIG. 2P). See, e.g., Tamaki et al. (2013) J. Clin. Invest. 123(10):4513-4524, herein incorporated by reference in its entirety for all purposes. Reduced glucose tolerance was not apparent in the R138X mice, and we did not observe changes in glucose-induced insulin secretion (FIGS. 2G-2H). Glucose tolerance and insulin sensitivity were similar in the R138X and control mice, and we did not observe changes in glucose-induced insulin secretion (FIGS. 2G, 2H, and 2M). In addition, the absence of islet zinc did not affect islet morphology as insulin and glucagon staining was not altered (FIGS. 2I-2K; FIGS. 6A-6B). In addition, the absence of islet zinc did not affect islet morphology as beta-cell and alpha-cell masses were not altered (FIGS. 2I, 2N, 6A, and 6E). Taken together, these data show that the R138X variant does not affect glucose homeostasis or glucose-induced insulin secretion in chow-fed mice.

TABLE 5
Body Weight of SLC30A8 KO and R138X Mice.

| Genotype | Sex | Diet | BW (g) |
|---|---|---|---|
| WT | Male | Chow | 26 ± 0.4 |
| R138K | Male | Chow | 27 ± 0.4 |
| WT | Male | Chow | 29 ± 1.1 |
| KO | Male | Chow | 27 ± 0.6 |
| WT | Male | HFD | 55 ± 1.9 |
| R138X | Male | HFD | 57 ± 0.9 |
| WT | Male | HFD | 53 ± 1.4 |
| KO | Male | HFD | 54 ± 1.2 |
| WT | Male | HFD | 51 ± 0.9 |
| R138 HET | Male | HFD | 48 ± 0.8 |
| WT | Female | HFD | 54 ± 4.2 |
| R138X | Female | HFD | 54 ± 2.1 |

TABLE 6
Body Composition of Chow-Fed SLC30A8 KO and R138X Mice.

| | | Lean | Fat | Bone | | Bone Mineral |
|---|---|---|---|---|---|---|
| | | Volume | Volume | Volume | Bone Density | Content |
| Genotype | Sex | (cm³) | (cm³) | (cm³) | (mgHA/cm³) | (mgHA) |
| WT | Male | 13.5 ± 0.4 | 2.6 ± 0.2 | 1.1 ± 0.03 | 283 ± 3.3 | 320 ± 12.4 |
| R138X | Male | 13.4 ± 0.3 | 3.1 ± 0.2 | 1.1 ± 0.11 | 281 ± 2.8 | 311 ± 6.6 |
| WT | Female | 13.7 ± 0.3 | 4.4 ± 0.5 | 1.2 ± 0.02 | 443 ± 3.4 | 525 ± 5.4 |
| R138X | Female | 13.8 ± 0.4 | 5.5 ± 0.5 | 1.2 ± 0.02 | 439 ± 5.2 | 519 ± 15.8 |
| WT | Male | 14.1 ± 0.3 | 4.4 ± 0.3 | 1.2 ± 0.01 | 294 ± 2.9 | 339 ± 6.3 |
| KO | Male | 13.9 ± 0.2 | 4.2 ± 0.4 | 1.2 ± 0.01 | 297 ± 2.9 | 343 ± 5.9 |

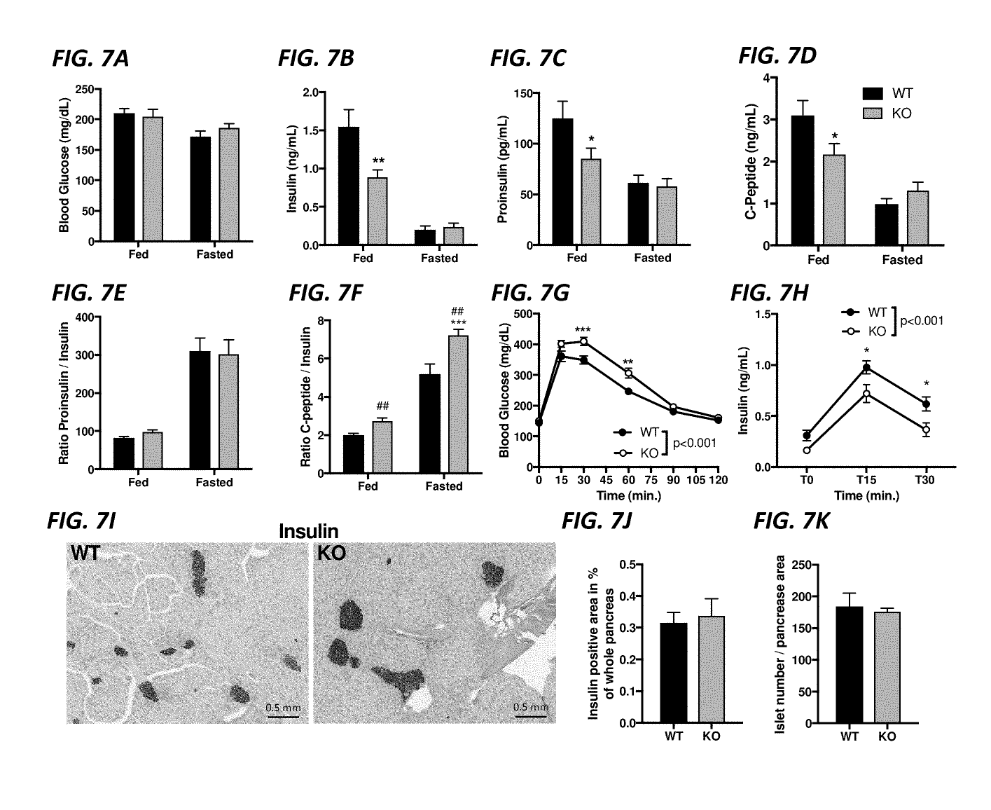

We also generated Slc30a8 KO mice in which the coding sequence from the Slc30a8 start codon to the Slc30a8 stop codon (i.e., the entire coding sequence) was deleted. We then phenotyped Slc30a8 KO mice on chow diet (FIGS. 7A-7K). In agreement with published results, the Slc30a8 KO mice showed mild impaired glucose tolerance and reduced glucose-induced plasma insulin levels (FIGS. 7B, 7G, 7H). See, e.g., Lemaire et al. (2009) Proc. Natl. Acad. Sci. U.S.A. 106(35):14872-14877; Nicolson et al. (2009) Diabetes 58(9):2070-2083; and Rutter et al. (2016) Proc. Nutr. Soc. 75(1):61-72, each of which is herein incorporated by reference in its entirety for all purposes. In addition, Slc30a8 KO mice had a higher ratio of C-peptide to insulin, suggesting increased insulin clearance similar to a recent report and presumably reflecting impaired zinc secretion from Slc30a8 KO islets. See, e.g., Tamaki et al. (2013) J. Clin. Invest. 123(10):4513-4524 and Mitchell et al. (2016) Mol. Endocrinol. 30(1):77-91, each of which is herein incorporated by reference in its entirety for all purposes.

Taken together, these data show that introduction of the R138X putative LOF variant has minor effects on proinsulin processing and does not alter glucose homeostasis or glucose-induced insulin secretion in mice maintained on regular chow diet. This R138X phenotype thus differs from that observed in Slc30a8 KO mice, which have impaired glucose tolerance and reduced glucose-induced plasma insulin levels when maintained on regular chow diet.

R138X Mice on High-Fat Diet have Increased Insulin Secretion and Beta-Cell Number.

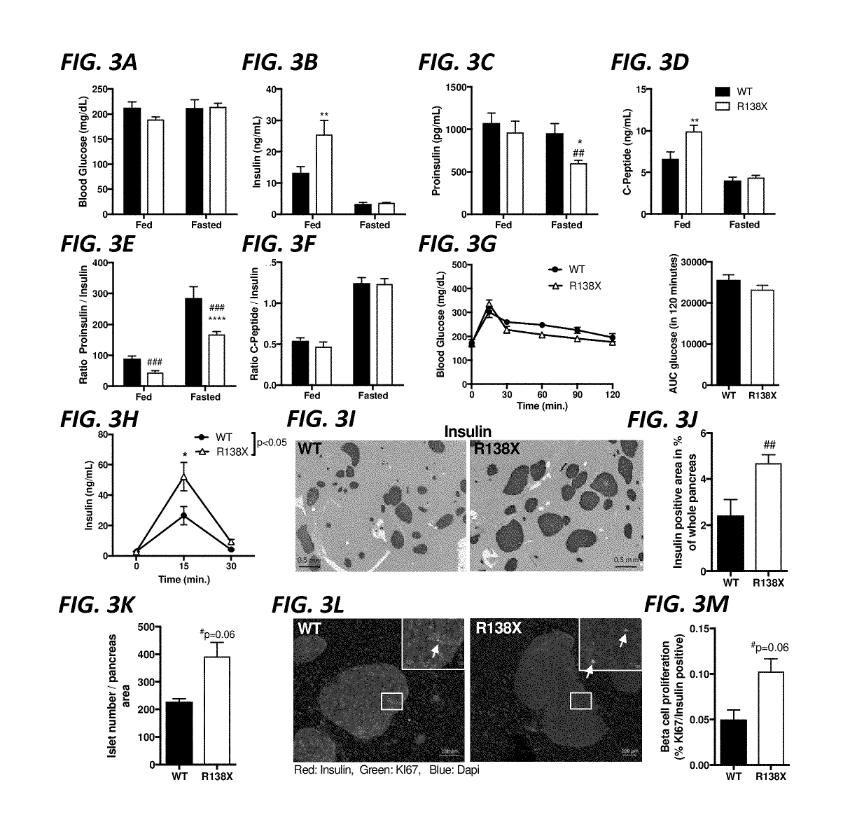

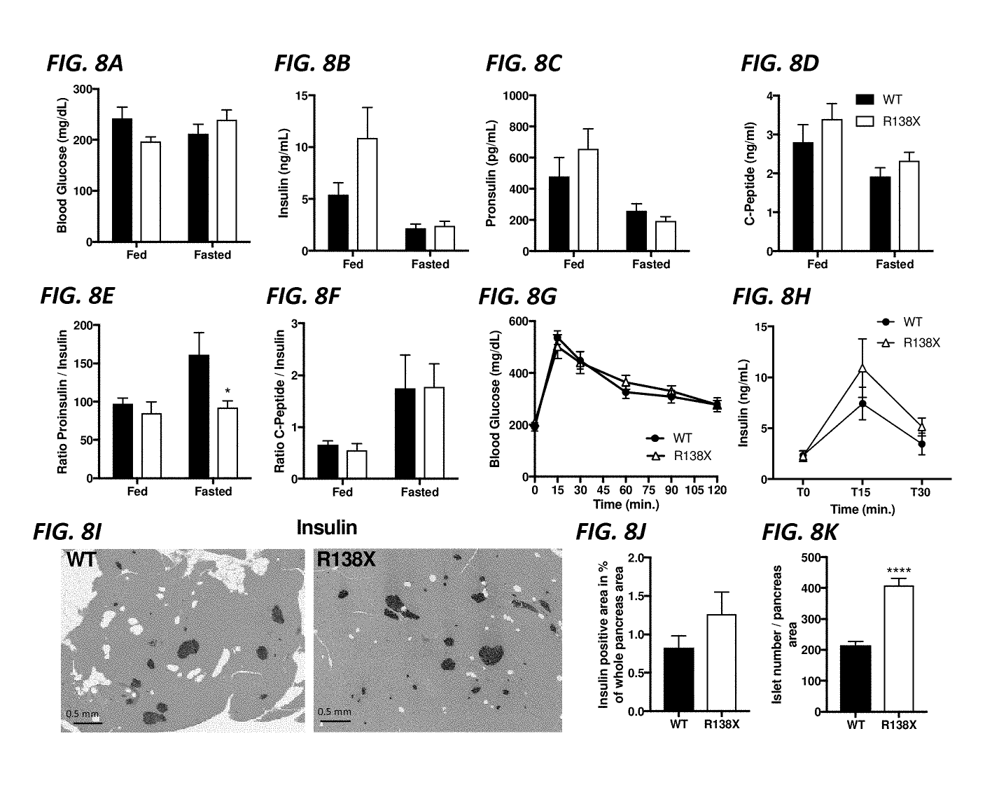

Since carriers of the human R138X are protected from development of T2D, we challenged the mice with a high-fat diet (HFD). Body weight and fed glucose levels did not differ between WT and R138X mice (Table 5, FIG. 3A). However, after 20 weeks on HFD we observed higher circulating insulin levels in fed R138X mice compared to WT mice (FIG. 3B). While circulating C-peptide levels mimicked the insulin levels, fasted proinsulin levels were lower in the R138X mice, resulting in a decreased proinsulin to insulin ratio (FIGS. 3C-3F). Despite the increase in fed insulin, glucose tolerance was not significantly improved in R138X mice (FIG. 3G). To examine insulin secretion in more detail, we measured circulating insulin after an oral glucose tolerance test (oGTT). In R138X mice, an oral glucose bolus doubled the amount of circulating insulin at 15 min when compared to WT mice (FIG. 3H). Strikingly, this was associated with an increased number of insulin-producing beta cells (FIG. 3I). Quantification of the insulin staining showed a doubling of the insulin-positive area, as well as the islet number (FIGS. 3J-3K). In contrast, we detected no change in the number of alpha cells using glucagon staining (FIGS. 6C-6D). Ki67 staining suggested that the increase in beta-cell area was secondary to enhanced beta-cell proliferation. The number of insulin- and Ki67-double positive cells increased two-fold in the pancreas of R138X mice compared to WT mice. A similar, although less pronounced, metabolic phenotype was observed in female R138X mice (FIGS. 8A-8K). After more than 20 weeks on HFD, female R138X mice showed a reduced proinsulin to insulin ratio when fasted, and an increase in circulating insulin levels associated with a higher number of islets (FIGS. 8B-8K).

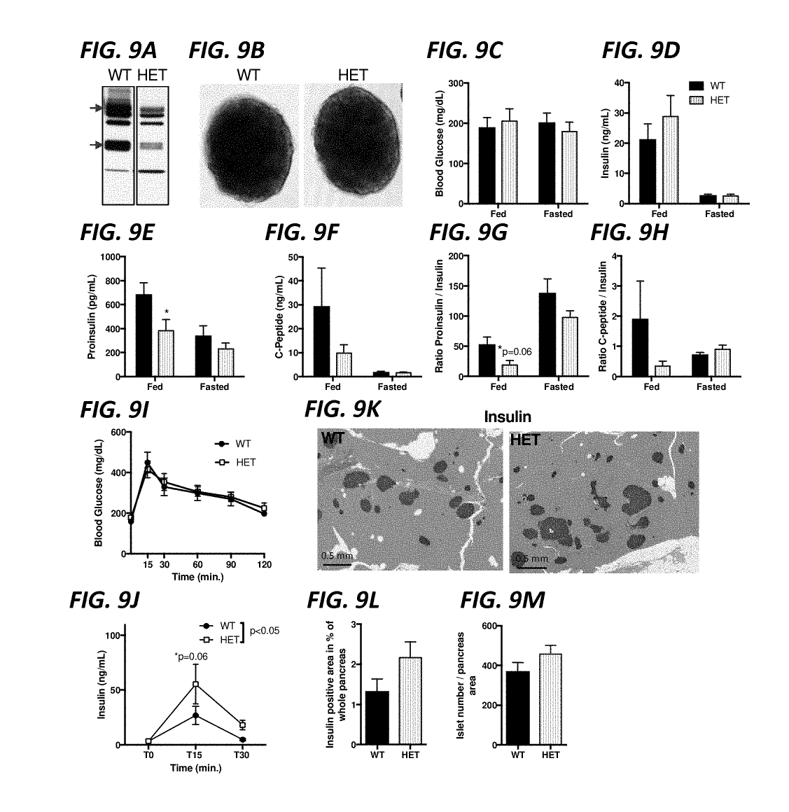

Because the protective phenotype of the R138X variant was observed in heterozygous carriers, we also analyzed heterozygous R138X mice on HFD (FIGS. 9A-9M). Western blot analysis showed a strong reduction of SLC30A8 protein in islets from heterozygous R138X mice (FIG. 9A), while islet zinc levels were comparable to WT mice when examined by dithizone staining (FIG. 9B). Although the phenotype was milder compared to the homozygous R138X mice, heterozygous R138X mice showed an increase in glucose-induced insulin secretion (FIG. 9J) associated with an expansion of beta-cell area after more than 20 weeks on HFD (FIG. 9K-9M). Taken together, these data suggest that HFD mice carrying the human R138X mutation have greater capacity to secrete insulin, which in part results from an increased number of beta cells.

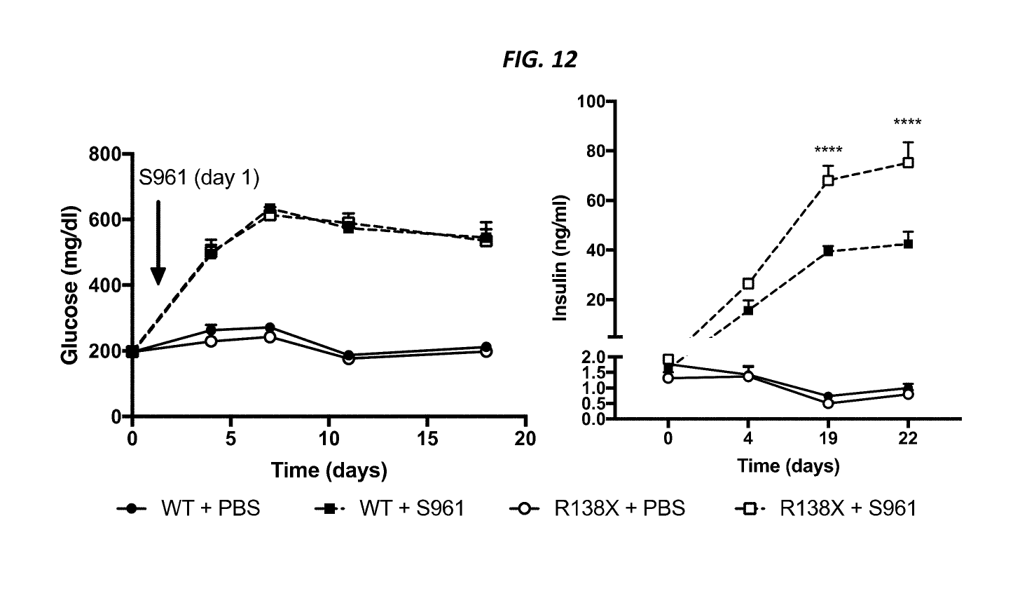

As an alternative way to induce insulin resistance and increase beta-cell insulin secretion, we used the insulin receptor antagonist S961 to block the insulin receptor in R138X mice and WT mice. Blockade of the insulin receptor increased circulating glucose and insulin levels as expected in both R138X mice and WT mice. However, we observed a doubling of the circulating insulin in R138X mice compared to WT mice. See FIG. 12. SLC30A8 KO mice do not show this increase in insulin secretion over WT mice (data not shown). These data are consistent with the data suggesting that HFD mice carrying the human R138X mutation have greater capacity to secrete insulin.

Slc30a8 KO Mice on High-Fat Diet do not have Increased Circulating Insulin Levels or Beta-Cell Number.

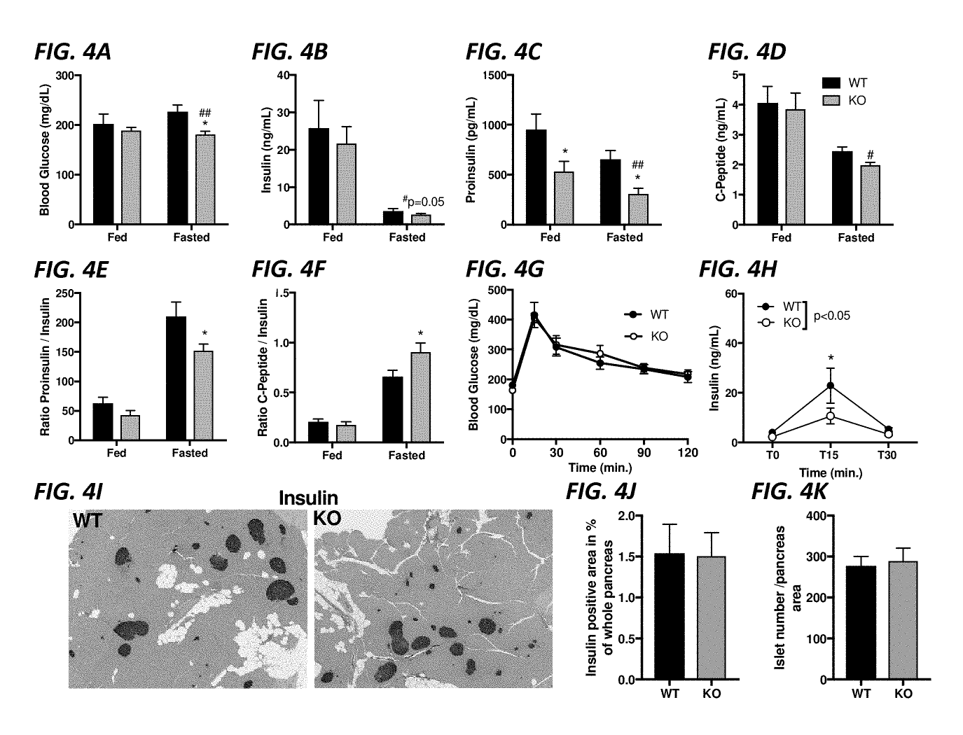

We also examined our Slc30a8 KO mice after 20 weeks on HFD (FIGS. 4A-4K). These mice did not show an increase in fed circulating insulin levels, but had reduced fasted insulin, proinsulin and C-peptide levels (FIGS. 4B-4D). Similar to the R138X mice, the proinsulin to insulin ratio was lower in the Slc30a8 KO mice (FIG. 4E). However, the C-peptide to insulin ratio was elevated in the Slc30a8 KO mice similar to what was observed on chow diet. While glucose tolerance was unchanged, Slc30a8 KO mice showed lower glucose-induced circulating insulin levels. In contrast to what we observed in the R138X mice, beta-cell number was unchanged in the Slc30a8 KO mice (FIGS. 4I-4K).

R138X Mice have Increased Insulin Secretion in Response to Insulin Receptor Inhibition-Induced Hyperglycemia.

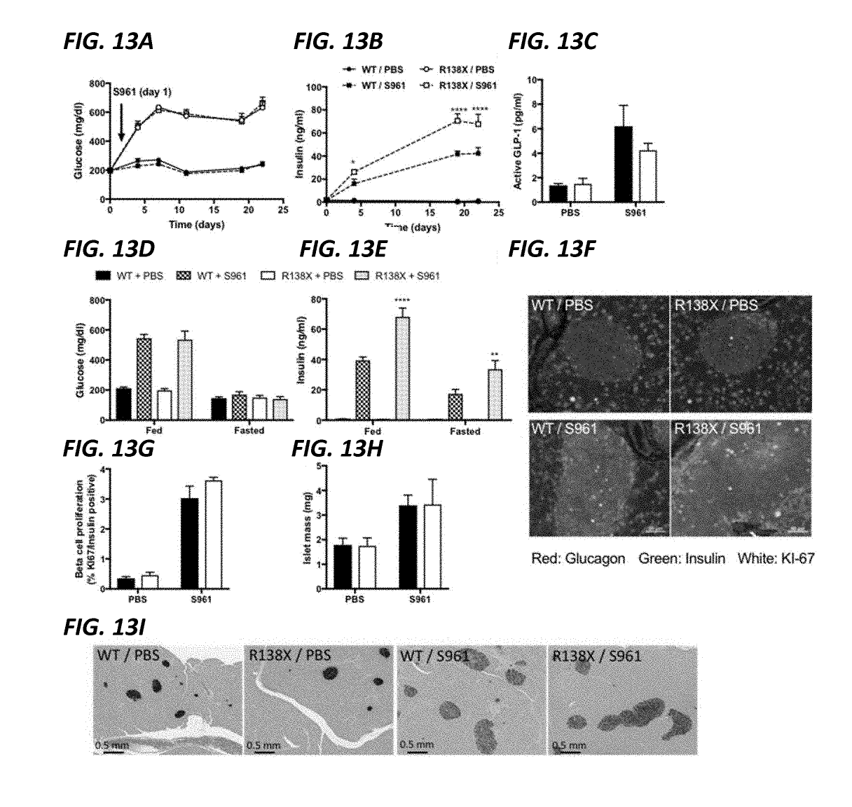

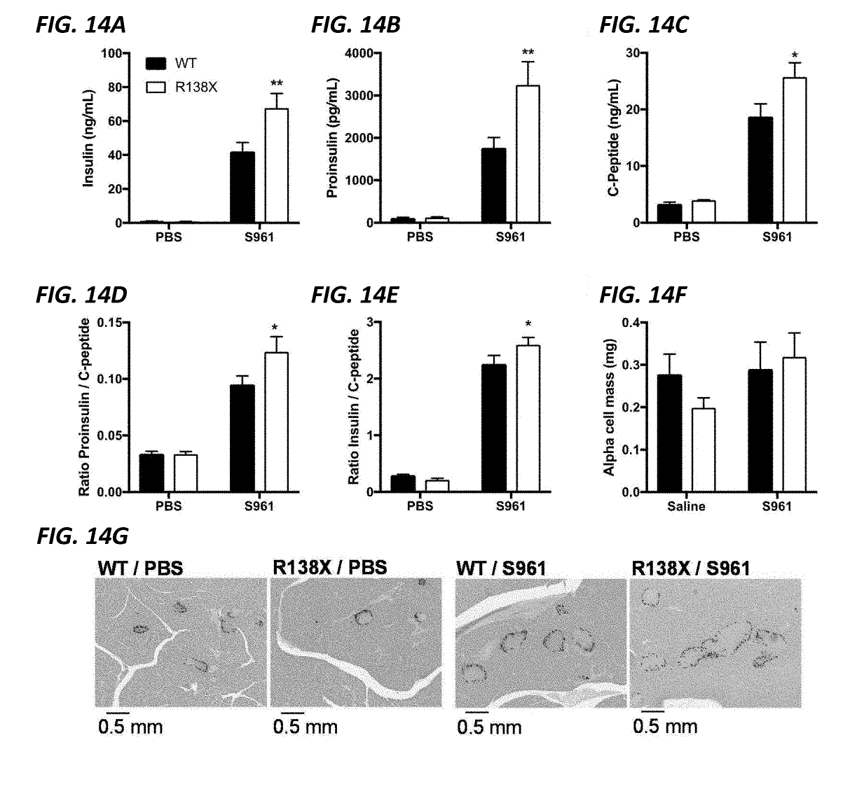

Next, we wanted to investigate a potential effect of the R138X mutation in metabolically challenged mice. We used the insulin receptor antagonist S961 to induce severe insulin resistance and hyperglycemia. See, e.g., Schaffer et al. (2008) Biochem. Biophys. Res. Commun. 376(2):380-383, herein incorporated by reference in its entirety for all purposes. As expected, S961 caused hyperglycemia (approximately 600 mg/dl) in all treated animals, while the control animals kept normal fed glucose levels (approximately 200 mg/dl) (FIG. 13A). Circulating insulin levels rose in response to S961 in all treated mice. Strikingly, R138X mice had more than 50% (WT: 42.33±5.03 ng/ml; R138X: 67.75±19.16 ng/ml) higher circulating insulin levels upon S961-induced hyperglycemia compared to S961-treated WT mice (FIG. 13B). The increase in plasma insulin was not a result of improved proinsulin processing or decreased insulin clearance (FIGS. 14A-14E), or changes in circulating GLP-1 levels between the R138X and WT mice (FIG. 13C). Circulating insulin was higher in fed and fasted condition in R138X mice treated with S961, but blood glucose levels did not differ between genotypes because of the inhibition of insulin action on the insulin receptor by S961 (FIGS. 13D-13E). Of note, fasted insulin levels were substantially elevated with S961 treatment in WT and R138X mice when compared to PBS-treated mice despite normal fasting blood glucose levels.

To investigate whether the higher insulin levels in the mutant mice were a consequence of increased beta-cell proliferation, we measured Ki-67/insulin positive beta cells. While beta-cell proliferation clearly increased with S961 treatment, there was no difference in beta-cell proliferation between R138X and WT mice (FIGS. 13F-13G). Consistent with the proliferation data, there was no difference in islet or alpha-cell mass in S961-treated WT and R138X mice (FIGS. 13F and 13H, FIGS. 14F-14G). Despite the large increase in blood insulin, beta cells from S961-treated R138X mice did not appear to be more insulin-depleted than beta cells from the S961-treated WT mice (FIG. 13F). In summary, these data suggest that islets from the R138X mice have higher capacity to secrete insulin when challenged with hyperglycemia.

Islets from R138X Mice have Decreased Mitochondrial Gene Expression and Increased Hvcn1 Expression.

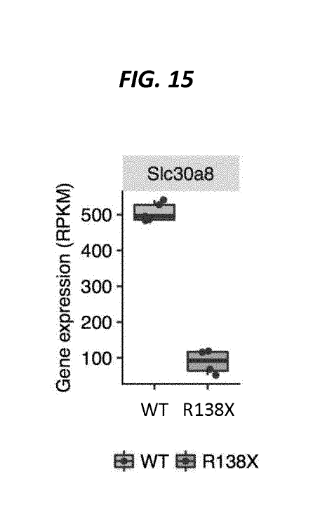

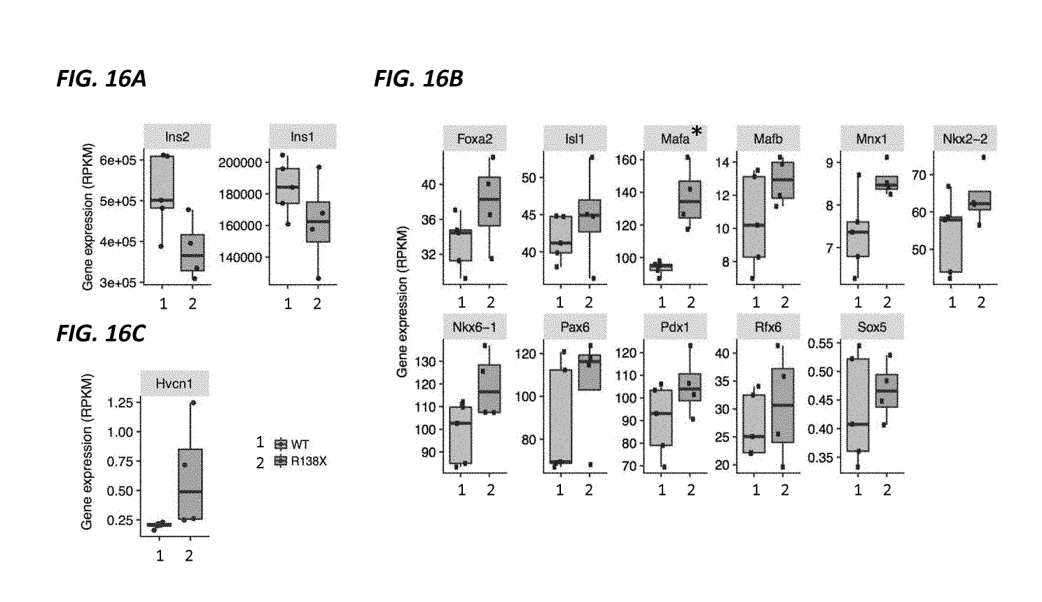

To understand the differences between WT and R138X islets in more detail, we performed RNA sequencing on islets isolated from chow-fed mice. In confirmation with our RNAscope and TAQMAN® data (FIGS. 1A-1B), the amount of Slc30a8 mRNA was strongly reduced (from approximately 500 RPKM in WT to approximately 100 RKPM in R138X), but not absent (FIG. 15). We found that 61 genes (19 genes increased, 43 genes decreased) were differentially regulated in R138X islets (Table 7). Interestingly, this gene list does not contain genes involved in zinc metabolism or insulin biosynthesis. However, the expression of insulin 2 (Ins2) was significantly down-regulated and the expression of insulin 1 (Ins1) trended lower too (FIG. 16A). This prompted us to look more carefully into the expression levels of beta-cell regulators, including transcription factors for insulin. As shown in FIG. 16B, Mafa was significantly upregulated in islets of R138X mice but had missed our cut-off of 1.5-fold. Since Mafa positively regulates insulin transcription and its expression is usually positively associated with the expression of the hormone, it does not explain the reduction the Ins2 gene expression. Out of the 43 down-regulated genes, 12 genes were mitochondrial proteins and 7 belonged to the group of Oxphos genes (Table 7). We therefore looked into the expression level of all Oxphos genes and noted that the expression of most of these genes was reduced (Table 8, Table 9). Complex I, III, and V were mainly affected with a reduction in gene expression of about 40%, suggesting a reduced capacity to generate ATP.

TABLE 7
Differentially Regulated Genes in Islets from Chow-Fed WT and
R138X Mice (Mitochondrial Genes Marked in Bold).

| Gene.ID | Symbol | Fold change | P value | Description |
|---|---|---|---|---|
| 170942 | Erdr1 | 3.34 | 9.80E−04 | erythroid differentiation regulator 1 |
| 74096 | Hvcn1 | 2.87 | 3.90E−03 | hydrogen voltage-gated channel 1 |
| 12483 | Cd22 | 2.79 | 2.70E−03 | CD22 antigen |
| 19354 | Rac2 | 2.63 | 6.60E−04 | RAS-related C3 botulinum substrate 2 |
| 12265 | Ciita | 2.47 | 2.50E−03 | class II transactivator |
| 17691 | Sik1 | 2.37 | 7.10E−08 | salt-inducible kinase 1 |
| 18636 | Cfp | 2.01 | 7.50E−03 | complement factor properdin |
| 17318 | Mid1 | 1.79 | 1.70E−03 | midline 1 |
| 170935 | Grid2ip | 1.75 | 8.60E−03 | glutamate receptor, ionotropic, delta 2 (Grid2) interacting protein 1 |
| 666048 | Trabd2b | 1.72 | 4.20E−03 | TraB domain-containing 2B |
| 11899 | Astn1 | 1.67 | 1.60E−03 | astrotactin 1 |
| 23984 | Pde10a | 1.66 | 4.50E−03 | phosphodiesterase 10A |
| 244723 | Olfm2 | 1.62 | 7.40E−03 | olfactomedin 2 |
| 17294 | Mest | 1.60 | 8.30E−04 | mesoderm specific transcript |
| 319504 | Nrcam | 1.56 | 6.10E−06 | neuron-glia-CAM-related cell adhesion molecule |
| 104601 | Mycbpap | 1.53 | 3.80E−03 | MYCBP associated protein |
| 12545 | Cdc7 | 1.53 | 8.00E−03 | cell division cycle 7 (S. cerevisiae) |
| 544617 | Arhgap27 | 1.52 | 3.50E−03 | Rho GTPase activating protein 27 |
| 319760 | D130020L05Rik | −1.52 | 1.10E−03 | RIKEN cDNA D130020L05 gene |
| 195531 | Gm13152 | −1.54 | 3.40E−03 | predicted gene 13152 |
| 66091 | Ndufa3 | −1.54 | 1.20E−03 | NADH dehydrogenase (ubiquinone) 1 alpha subcomplex, 3 |
| 16334 | Ins2 | −1.54 | 7.20E−03 | insulin II |
| 235043 | Tmem205 | −1.54 | 8.70E−03 | transmembrane protein 205 |
| 14470 | Rabac1 | −1.56 | 2.90E−03 | Rab acceptor 1 (prenylated) |
| 14776 | Gpx2 | −1.59 | 1.80E−07 | glutathione peroxidase 2 |
| 66416 | Ndufa7 | −1.59 | 6.20E−03 | NADH dehydrogenase (ubiquinone) 1 alpha subcomplex, 7 (B14.5a) |
| 228715 | Gm561 | −1.59 | 6.90E−03 | predicted gene 561 |
| 66845 | Mrpl33 | −1.59 | 2.10E−03 | mitochondrial ribosomal protein L33 |
| 17178 | Fxyd3 | −1.59 | 3.10E−03 | FXYD domain-containing ion transport regulator 3 |
| 20832 | Ssr4 | −1.59 | 6.40E−03 | signal sequence receptor, delta |
| 66594 | Uqcr11 | −1.61 | 7.50E−03 | ubiquinol-cytochrome c reductase, complex III subunit XI |
| 66152 | Uqcr10 | −1.61 | 7.30E−03 | ubiquinol-cytochrome c reductase, complex III subunit X |
| 67267 | Uqcc2 | −1.61 | 7.60E−03 | ubiquinol-cytochrome c reductase complex assembly factor 2 |
| 69038 | Tmem258 | −1.61 | 4.60E−03 | transmembrane protein 258 |
| 67885 | 1500011K16Rik | −1.61 | 9.30E−03 | RIKEN cDNA 1500011K16 gene |
| 100502825 | Rpl37rt | −1.61 | 5.40E−03 | predicted gene 13826 |
| 66117 | 1110001J03Rik | −1.61 | 4.40E−03 | RIKEN cDNA 1110001J03 gene |
| 69094 | Tmem160 | −1.61 | 8.50E−03 | transmembrane protein 160 |
| 67941 | Rps27l | −1.61 | 4.00E−03 | ribosomal protein S27-like |
| 234421 | Cib3 | −1.64 | 5.30E−03 | calcium and integrin binding family member 3 |
| 11807 | Apoa2 | −1.67 | 3.80E−04 | apolipoprotein A-II |
| 20892 | Stra13 | −1.67 | 2.90E−03 | stimulated by retinoic acid 13 |
| 66477 | Usmg5 | −1.67 | 5.60E−03 | upregulated during skeletal muscle growth 5 |
| 68563 | Dpm3 | −1.67 | 2.50E−03 | dolichyl-phosphate mannosyltransferase polypeptide 3 |
| 22177 | Tyrobp | −1.67 | 9.90E−03 | TYRO protein tyrosine kinase binding protein |
| 69386 | Hist1h4h | −1.69 | 5.00E−03 | histone cluster 1, H4h |
| 268686 | S100z | −1.69 | 9.90E−04 | S100 calcium binding protein, zeta |
| 73720 | Cst6 | −1.69 | 2.80E−03 | cystatin E/M |
| 27425 | Atp5l | −1.69 | 4.90E−03 | ATP synthase, H+ transporting, mitochondrial F0 complex, subunit G |
| 68194 | Ndufb4 | −1.72 | 4.70E−03 | NADH dehydrogenase (ubiquinone) 1 beta subcomplex 4 |
| 449000 | Zfp960 | −1.72 | 1.30E−03 | zinc finger protein 960 |
| 93757 | Immp2l | −1.75 | 3.20E−03 | IMP2 inner mitochondrial membrane peptidase-like [symbol] |
| 19735 | Rgs2 | −1.82 | 7.40E−03 | regulator of G-protein signaling 2 |
| 17873 | Gadd45b | −1.82 | 1.40E−04 | growth arrest and DNA-damage-inducible 45 beta |
| 20335 | Sec61g | −1.82 | 3.20E−03 | SEC61, gamma subunit |
| 13190 | Dct | −1.89 | 4.10E−04 | dopachrome tautomerase |
| 11811 | Apobec2 | −1.89 | 3.30E−03 | apolipoprotein B mRNA editing enzyme, catalytic polypeptide 2 |
| 100169864 | SlcUbl4a | −2.13 | 2.90E−05 | Slc10a3-Ubl4 read-through |
| 19782 | Rmrp | −2.17 | 8.40E−03 | RNA component of mitochondrial RNase P |
| 100568459 | Bc1 | −2.27 | 4.10E−03 | brain cytoplasmic RNA 1 |
| 239436 | Slc30a8 | −6.67 | 1.40E−43 | solute carrier family 30 (zinc transporter), member 8 |

TABLE 8
Summary of Oxphos Genes Expression Changes in Islets of WT and
R138X Mice.

| Complex | Total | Significantly Regulated (p < 0.05) | % Regulated |
|---|---|---|---|
| Complex I | 43 | 19 | 44.19 |
| Complex II | 4 | 1 | 25.00 |
| Complex III | 10 | 4 | 40.00 |
| Complex IV | 19 | 5 | 26.32 |
| Complex V | 20 | 9 | 45.00 |

TABLE 9
Change in Expression of All Oxphos Genes in Isolated Islets from WT
and R138X Mice (Genes that Changed Significantly in Bold).

| | R138X.vs. | R138X.vs. | | |
|---|---|---|---|---|
| | WT_islets. | WT_islets. | | Mitochondrial |
| symbol | fc | pval | Name | complex |
| MT-ND1 | 0.85 | 0.42 | mitochondrially encoded NADH:ubiquinone oxidoreductase core subunit 1 | 1 |
| MT-ND2 | 0.93 | 0.67 | mitochondrially encoded NADH:ubiquinone oxidoreductase core subunit 2 | 1 |
| MT-ND3 | 0.81 | 0.27 | mitochondrially encoded NADH:ubiquinone oxidoreductase core subunit 3 | 1 |
| MT-ND4 | 0.84 | 0.36 | mitochondrially encoded NADH:ubiquinone oxidoreductase core subunit 4 | 1 |
| MT-ND4L | 0.8 | 0.23 | mitochondrially encoded NADH:ubiquinone oxidoreductase core subunit 4L | 1 |
| MT-ND5 | 0.91 | 0.59 | mitochondrially encoded NADH:ubiquinone oxidoreductase core subunit 5 | 1 |
| MT-ND6 | 0.77 | 0.066 | mitochondrially encoded NADH:ubiquinone oxidoreductase core subunit 6 | 1 |
| NDUFS1 | 0.95 | 0.54 | NADH:ubiquinone oxidoreductase core subunit S1 | 1 |
| NDUFS2 | 0.79 | 0.0077 | NADH:ubiquinone oxidoreductase core subunit S2 | 1 |
| NDUFS3 | 0.86 | 0.18 | NADH:ubiquinone oxidoreductase core subunit S3 | 1 |
| NDUFS7 | 0.79 | 0.081 | NADH:ubiquinone oxidoreductase core subunit S7 | 1 |
| NDUFS8 | 0.75 | 0.055 | NADH:ubiquinone oxidoreductase core subunit S8 | 1 |
| NDUFV1 | 0.89 | 0.15 | NADH:ubiquinone oxidoreductase core subunit V1 | 1 |
| NDUFV2 | 0.8 | 0.2 | NADH:ubiquinone oxidoreductase core subunit V2 | 1 |
| NDUFAB1 | 0.81 | 0.058 | NADH dehydrogenase (ubiquinone) 1, alpha/beta subcomplex, 1 | 1 |
| NDUFA1 | 0.68 | 0.033 | NADH dehydrogenase (ubiquinone) 1 alpha subcomplex, 1 | 1 |
| NDUFA2 | 0.64 | 0.023 | NADH dehydrogenase (ubiquinone) 1 alpha subcomplex, 2 | 1 |
| NDUFA3 | 0.65 | 0.0012 | NADH dehydrogenase (ubiquinone) 1 alpha subcomplex, 3 | 1 |
| NDUFA5 | 0.72 | 0.021 | NADH dehydrogenase (ubiquinone) 1 alpha subcomplex, 5 | 1 |
| NDUFA6 | 0.76 | 0.098 | NADH dehydrogenase (ubiquinone) 1 alpha subcomplex, 6 (B14) | 1 |
| NDUFA7 | 0.63 | 0.0062 | NADH dehydrogenase (ubiquinone) 1 alpha subcomplex, 7 (B14.5a) | 1 |
| NDUFA8 | 0.91 | 0.35 | NADH dehydrogenase (ubiquinone) 1 alpha subcomplex, 8 | 1 |
| NDUFA9 | 0.87 | 0.22 | NADH dehydrogenase (ubiquinone) 1 alpha subcomplex, 9 | 1 |
| NDUFA10 | 0.91 | 0.15 | NADH dehydrogenase (ubiquinone) 1 alpha subcomplex 10 | 1 |
| NDUFA11 | 0.71 | 0.016 | NADH dehydrogenase (ubiquinone) 1 alpha subcomplex 11 | 1 |
| NDUFA12 | 0.69 | 0.017 | NADH dehydrogenase (ubiquinone) 1 alpha subcomplex, 12 | 1 |
| NDUFA13 | 0.7 | 0.01 | NADH dehydrogenase (ubiquinone) 1 alpha subcomplex, 13 | 1 |
| NDUFB2 | 0.55 | 0.012 | NADH dehydrogenase (ubiquinone) 1 beta subcomplex, 2 | 1 |
| NDUFB3 | 0.82 | 0.17 | NADH dehydrogenase (ubiquinone) 1 beta subcomplex 3 | 1 |
| NDUFB4 | 0.58 | 0.0047 | NADH dehydrogenase (ubiquinone) 1 beta subcomplex 4 | 1 |
| NDUFB5 | 0.8 | 0.11 | NADH dehydrogenase (ubiquinone) 1 beta subcomplex, 5 | 1 |
| NDUFB6 | 0.76 | 0.047 | NADH dehydrogenase (ubiquinone) 1 beta subcomplex, 6 | 1 |
| NDUFB7 | 0.79 | 0.12 | NADH dehydrogenase (ubiquinone) 1 beta subcomplex, 7 | 1 |
| NDUFB8 | 0.72 | 0.022 | NADH dehydrogenase (ubiquinone) 1 beta subcomplex 8 | 1 |
| NDUFB9 | 0.74 | 0.045 | NADH dehydrogenase (ubiquinone) 1 beta subcomplex, 9 | 1 |
| NDUFB10 | 0.73 | 0.027 | NADH dehydrogenase (ubiquinone) 1 beta subcomplex, 10 | 1 |
| NDUFB11 | 0.7 | 0.048 | NADH dehydrogenase (ubiquinone) 1 beta subcomplex, 11 | 1 |
| NDUFC1 | 0.73 | 0.03 | NADH dehydrogenase (ubiquinone) 1, subcomplex unknown, 1 | 1 |
| NDUFC2 | 0.75 | 0.074 | NADH dehydrogenase (ubiquinone) 1, subcomplex unknown, 2 | 1 |
| NDUFS4 | 0.76 | 0.039 | NADH dehydrogenase (ubiquinone) Fe—S protein 4 | 1 |
| NDUFS5 | NaN | NaN | NADH dehydrogenase (ubiquinone) Fe—S protein 5 | 1 |
| NDUFS6 | 0.72 | 0.017 | NADH dehydrogenase (ubiquinone) Fe—S protein 6 | 1 |
| NDUFV3 | 0.78 | 0.056 | NADH dehydrogenase (ubiquinone) flavoprotein 3 | 1 |
| SDHA | 0.97 | 0.52 | succinate dehydrogenase complex flavoprotein subunit A | 2 |
| SDHB | 0.75 | 0.031 | succinate dehydrogenase complex iron sulfur subunit B | 2 |
| SDHC | 0.91 | 0.22 | succinate dehydrogenase complex subunit C | 2 |
| SDHD | 0.81 | 0.12 | succinate dehydrogenase complex subunit D | 2 |
| CYC1 | 0.91 | 0.37 | cytochrome c1 | 3 |
| MT-CYB | 0.89 | 0.5 | mitochondrially encoded cytochrome b | 3 |
| UQCRB | 0.77 | 0.11 | ubiquinol-cytochrome c reductase binding protein | 3 |
| UQCRC1 | 0.93 | 0.48 | ubiquinol-cytochrome c reductase core protein 1 | 3 |
| UQCRC2 | 0.83 | 0.12 | ubiquinol-cytochrome c reductase core protein 2 | 3 |
| UQCRFS1 | 0.94 | 0.55 | ubiquinol-cytochrome c reductase, Rieske iron-sulfur polypeptide 1 | 3 |
| UQCRH | 0.64 | 0.016 | ubiquinol-cytochrome c reductase hinge protein | 3 |
| UQCRQ | 0.66 | 0.021 | ubiquinol-cytochrome c reductase complex III subunit VII | 3 |
| UQCR10 | 0.62 | 0.0073 | ubiquinol-cytochrome c reductase, complex III subunit X | 3 |
| UQCR11 | 0.62 | 0.0075 | ubiquinol-cytochrome c reductase, complex III subunit XI | 3 |
| COX4I1 | 0.73 | 0.058 | cytochrome c oxidase subunit 4I1 | 4 |
| C0X4I2 | 0.75 | 0.21 | cytochrome c oxidase subunit 412 | 4 |
| COX5A | 0.91 | 0.37 | cytochrome c oxidase subunit 5A | 4 |
| COX5B | 0.7 | 0.01 | cytochrome c oxidase subunit 5B | 4 |
| COX6A1 | 0.8 | 0.059 | cytochrome c oxidase subunit 6A1 | 4 |
| COX6A2 | 0.85 | 0.077 | cytochrome c oxidase subunit 6A2 | 4 |
| COX6B1 | 0.71 | 0.011 | cytochrome c oxidase subunit 6B1 | 4 |
| COX6B2 | NaN | NaN | cytochrome c oxidase subunit 6B2 | 4 |
| COX6C | 0.67 | 0.033 | cytochrome c oxidase subunit 6C | 4 |
| COX7A1 | NaN | NaN | cytochrome c oxidase subunit 7A1 | 4 |
| COX7A2 | 0.62 | 0.017 | cytochrome c oxidase subunit 7A2 | 4 |
| COX7B | 0.74 | 0.072 | cytochrome c oxidase subunit 7B | 4 |
| COX7B2 | NaN | NaN | cytochrome c oxidase subunit 7B2 | 4 |
| COX7C | 0.65 | 0.013 | cytochrome c oxidase subunit 7C | 4 |
| COX8A | 0.84 | 0.12 | cytochrome c oxidase subunit 8A | 4 |
| COX8C | NaN | NaN | cytochrome c oxidase subunit 8C | 4 |
| MT-CO1 | 0.87 | 0.45 | mitochondrially encoded cytochrome c oxidase I | 4 |
| MT-CO2 | 0.9 | 0.55 | mitochondrially encoded cytochrome c oxidase II | 4 |
| MT-CO3 | 0.77 | 0.2 | mitochondrially encoded cytochrome c oxidase III | 4 |
| ATP5F1A | 0.93 | 0.43 | ATP synthase F1 subunit alpha | 5 |
| ATP5F1B | 0.88 | 0.11 | ATP synthase F1 subunit beta | 5 |
| ATP5F1C | 0.82 | 0.13 | ATP synthase F1 subunit gamma | 5 |
| ATP5F1D | 0.85 | 0.16 | ATP synthase F1 subunit delta | 5 |
| ATP5F1E | 0.63 | 0.12 | ATP synthase F1 subunit epsilon | 5 |
| ATP5MC1 | 0.76 | 0.01 | ATP synthase membrane subunit c locus 1 | 5 |
| ATP5MC2 | 0.72 | 0.026 | ATP synthase membrane subunit c locus 2 | 5 |
| ATP5MC3 | 0.87 | 0.19 | ATP synthase membrane subunit c locus 3 | 5 |
| ATP5MD | 0.6 | 0.0056 | ATP synthase membrane subunit DAPIT | 5 |
| ATP5ME | 0.67 | 0.016 | ATP synthase membrane subunit e | 5 |
| ATP5MF | 0.64 | 0.022 | ATP synthase membrane subunit f | 5 |
| ATP5MG | 0.59 | 0.0049 | ATP synthase membrane subunit g | 5 |
| ATP5MPL | 0.68 | 0.03 | ATP synthase membrane subunit 6.8PL | 5 |
| MT-ATP6 | 0.82 | 0.32 | mitochondrially encoded ATP synthase membrane subunit 6 | 5 |
| MT-ATP8 | 0.82 | 0.36 | mitochondrially encoded ATP synthase membrane subunit 8 | 5 |
| ATP5PB | 0.84 | 0.18 | ATP synthase peripheral stalk-membrane subunit b | 5 |
| ATP5PD | 0.67 | 0.0081 | ATP synthase peripheral stalk subunit d | 5 |
| ATP5PF | 0.8 | 0.11 | ATP synthase peripheral stalk subunit F6 | 5 |
| ATP5PO | 0.78 | 0.046 | ATP synthase peripheral stalk subunit OSCP | 5 |
| ATP5IF1 | 0.74 | 0.07 | ATP synthase inhibitory factor subunit 1 | 5 |

Of note, the expression of the voltage-gated proton channel Hv1 (Hvcn1, alternate names VSOP, HV1) was upregulated in islets from R138X mice. Since this channel is Zn²⁺-regulated and present in the insulin secretory granules, and loss of Hvcn1 expression impairs insulin secretion in vivo and in vitro, this might be important for the observed phenotype in the R138X mice. See, e.g., Zhao et al. (2015) Biochem. Biophys. Res. Commun. 468(4):746-751; Wang et al. (2017) “The Voltage-gated Proton Channel Hv1 Is Required for Insulin Secretion in Pancreatic β Cells,” bioRxiv 097816, doi.org/10.1101/097816; and Qiu et al. (2016) Proc. Natl. Acad. Sci. U.S.A. 113(40):E5962-E5971, each of which is herein incorporated by reference in its entirety for all purposes.

### Discussion

In this study, we generated a new Slc30a8 putative LOF mouse model (R138X mice) mimicking one of the two most common human SLC30A8 putative LOFs (p.Arg138*). See, e.g., Flannick et al. (2014) Nat. Genet. 46(4):357-363, herein incorporated by reference in its entirety for all purposes. Data obtained from these mice can for the first time explain why SLC30A8 putative LOF mutations in humans are protective against development of T2D. Introduction of the R138X mutation resulted in an increased number of beta cells allowing for higher insulin secretion in an insulin resistant state. These data indicate that R138X beta cells can therefore better compensate for the increased insulin demand preventing and/or delaying the failure of beta cells and onset of T2D. Using this model, we show that the introduction of the R138X mutation results in the secretion of 50% more insulin in response to hyperglycemia. These data suggest that the protection from T2D in humans is mediated, at least in part, by an increase in the insulin secretory capacity allowing the beta cells to compensate for enhanced insulin demand thereby preventing or delaying the failure of these cells and onset of T2D. The present approach thus demonstrates the feasibility of using mouse genetics to explore the mechanisms of how identified human T2D risk or protective genes act. The present approach also emphasizes the utility of mouse genetics to explore the mechanisms of genetic variants which affect the risk of T2D in humans.

More than eight different Slc30a8 knockout mouse models have been reported. See, e.g., Lemaire et al. (2009) Proc. Natl. Acad. Sci. U.S.A. 106(35):14872-14877; Nicolson et al. (2009) Diabetes 58(9):2070-2083; Tamaki et al. (2013) J. Clin. Invest. 123(10):4513-4524; Pound et al. (2009) Biochem. J. 421(3):371-376; Pound et al. (2012) PLoS One 7(7):e40972; Wijesekara et al. (2010) Diabetologia 53(8):1656-1668; and Hardy et al. (2012) Am. J. Physiol. Endocrinol. Metab. 302(9):E1084-E1096, each of which is herein incorporated by reference in its entirety for all purposes. These models vary in genetic background, Slc30a8 targeting strategy, and the specific Cre-line that was used when mice with beta-cell-specific deletions were generated. The metabolic phenotypes of these mice vary too. Differences in body weight gain, glucose tolerance and insulin secretion have been reported on chow and high-fat diet (HFD). See, e.g., Rutter et al. (2016) Proc. Nutr. Soc. 75(1):61-72, herein incorporated by reference in its entirety for all purposes. However, all published Slc30a8 knockout phenotypes point towards a worsening of glucose control with impaired glucose tolerance observed in at least six publications. For comparative reasons, we also generated a Slc30a8 KO mouse line on 100% C57BL/6 background, same as the R138X. This knockout mouse phenocopied to a large extend the published data showing impaired glucose tolerance (on chow diet), lower glucose-induced insulin secretion (on chow and HFD), and impaired insulin clearance (on chow and HFD). On the contrary, we did not observe a major metabolic phenotype, including worsening of glucose control in the R138X mice on chow diet. The difference between the knockout and the R138X mouse lines became even more apparent after more than 20 weeks on HFD. Insulin secretion and the amount of beta cells was increased in R138X mice, but not in Slc30a8 KO mice. The late appearance of the observed phenotype suggests that the compensatory increase in beta-cell proliferation and number happened rather late in HFD feeding. However, we cannot exclude the possibility that the number of beta cells differed already after 10 weeks on HFD when no change in insulin secretion was observed. Hardy et al. observed an increase in the number of beta cells and circulating insulin in their global Slc30a8 KO mouse after a HFD treatment of 16 weeks. See, e.g., Hardy et al. (2012) Am. J. Physiol. Endocrinol. Metab. 302(9):E1084-E1096, herein incorporated by reference in its entirety for all purposes. However, this was most likely a consequence of the deteriorated glucose control with marked obesity, hyperglycemia, and enhanced insulin resistance in their KO mice compared to wild type mice, an effect not observed in the present study. Importantly, while Hardy et al. observed an increase in proinsulin to insulin ratio in the Slc30a8 KO mice, we observed a decrease in this ratio in the R138X mice on HFD, suggesting that the beta cells had improved insulin processing and better health than the WT beta cells. See, e.g., Hardy et al. (2012) Am. J. Physiol. Endocrinol. Metab. 302(9):E1084-E1096, herein incorporated by reference in its entirety for all purposes. Interestingly, the improvement in proinsulin to insulin ratio was also observed in our Slc30a8 KO mice, showing that knockout and R138X mice do not differ in all metabolic aspects. Since a high proinsulin to insulin ratio is associated with progression to T2D in humans, this could also contribute to SLC30A8 LOF-mediated protection from T2D. See, e.g., Kirchhoff et al. (2008) Diabetologia 51(4):597-601; Loopstra-Masters et al. (2011) Diabetologia 54(12):3047-3054; and Saad et al. (1990) J. Clin. Endocrinol. Metab. 70(5):1247-1253, each of which is herein incorporated by reference in its entirety for all purposes.

All Slc30a8 KO phenotypes show either no change or a decrease in circulating insulin, which was in some instances associated with worsening of glucose tolerance. See, e.g., Nicolson et al., (2009) Diabetes 58(9):2070-2083; Pound et al. (2009) Biochem. J. 421(3):371-376; and Mitchell et al. (2016) Mol. Endocrinol. 30(1):77-91, each of which is herein incorporated by reference in its entirety for all purposes. On the contrary, an increase in in vitro insulin secretion has been observed in islets from three KO models. See, e.g., Nicolson et al., (2009) Diabetes 58(9):2070-2083; Tamaki et al. (2013) J. Clin. Invest. 123(10):4513-4524; and Hardy et al. (2012) Am. J. Physiol. Endocrinol. Metab. 302(9):E1084-E1096, each of which is herein incorporated by reference in its entirety for all purposes. We did not observe a major metabolic phenotype, including a change in glucose-induced insulin secretion, in chow-fed R138X mice. When challenged with S961, however, these mice were able to secrete much more insulin than their WT counterparts. This increase in insulin secretion was not secondary to an increase in beta-cell mass and has not been reported in any Slc30a8 KO model.

It is unclear why the metabolic phenotype of the R138X mice differs so much from the Slc30a8 KO mice, but the data suggest a potential gain of function for the R138X allele. Currently, it is unclear whether the R138X mice are like Slc30a8 KO mice, since we cannot rule out that the remaining mRNA is translated into a truncated protein. We were not able to detect a truncated version of the SLC30A8 protein that could readily explain phenotype differences in the islets of the R138X mice. However, the RNA is clearly present, and overexpressed R138X protein was detected in HEK293 cells and accumulated by proteasomal inhibition (FIG. 5), which is contrary to what was previously observed in HeLa cells. See, e.g., Flannick et al. (2014) Nat. Genet. 46(4):357-363, herein incorporated by reference in its entirety for all purposes. Thus, we cannot exclude the existence of low levels of protein, perhaps in a subset of the islet cells from the R138X mice. Independent of the potential expression of SLC30A8 protein in R138X mice, dithizone staining clearly indicated that the introduction of the R138X putative LOF mutation resulted in a loss of islet zinc accumulation consistent with what is described in the Slc30a8 KO mice. Another possibility is that the truncated Slc30a8 mRNA, while no longer coding for a peptide, now serves a role as a long non-coding RNA, affecting the transcription of related genes (possibly including other Slc30a family members) in trans. See, e.g., Batista and Chang (2013) Cell 152(6):1298-1307, herein incorporated by reference in its entirety for all purposes.

Changes in subcellular free Zn²⁺ may lead to increased proliferation and/or insulin secretion for a number of reasons. A plethora of intracellular signaling systems are affected by free Zn²⁺ in mammalian cells with almost 10% of the cellular proteome able to bind these ions. See, e.g., Rutter et al. (2016) Proc. Nutr. Soc. 75(1):61-72, herein incorporated by reference in its entirety for all purposes. Although free Zn²⁺ concentrations are likely to be lowered globally in the cytosol by Slc30a8 inactivation in the beta cell, highly localized increases in free Zn²⁺ close to the plasma membrane in R138X beta cells might enhance signaling by receptor tyrosine kinases (e.g. insulin or IGF1 receptors) by inhibiting protein tyrosine phosphatases, thus promoting cell growth. See, e.g., Gerber et al. (2014) Diabetologia 57(8):1635-1644 and Bellomo et al. (2014) Metallomics 6(7):1229-1239, each of which is herein incorporated by reference in its entirety for all purposes. On the other hand, globally lowered cytosolic Zn²⁺ levels are expected to reduce apoptotic rates, for example by interaction with caspases. See, e.g., Fukamachi et al. (1998) Biochem. Biophys. Res. Commun. 246(2):364-369, herein incorporated by reference in its entirety for all purposes.

Insulin secretion may be upregulated in R138X mice through upregulation of the voltage-gated proton channel Hvcn1. Hvcn1 is present in secretory granules and inhibited by zinc ions. See, e.g., Qiu et al. (2016) Proc. Nat. Acad. Sci. U.S.A. 113(40):E5962-E5971, herein incorporated by reference in its entirety for all purposes. In addition, Hvcn1 KO mice have decreased insulin secretion and impaired glucose control. Wang et al. (2017) “The Voltage-gated Proton Channel Hv1 Is Required for Insulin Secretion in Pancreatic β Cells,” bioRxiv 097816, doi.org/10.1101/097816, herein incorporated by reference in its entirety for all purposes. Since islets from the R138X mice are zinc-depleted, one would expect an increase in the activity of this channel. On top of that, we see a 3-fold upregulation of the Hvcn1 transcript in islets from R138X mice. While these data suggest that the increase in insulin secretion is at least in part mediated by Hvcn1, further experiments have to validate this hypothesis. Another interesting finding is the reduction of mitochondrial gene expression in R138X mice. This seems rather counterintuitive, since ATP synthesis is required for proper insulin secretion. See, e.g., Wiederkehr and Wollheim (2012) Mol. Cell. Endocrinol. 353(1-2):128-137, herein incorporated by reference in its entirety for all purposes. However, a recent study described an alternative pathway to activate insulin secretion in beta cells. See, e.g., De Marchi et al. (2017) J. Cell. Sci. 130(11):1929-1939, herein incorporated by reference in its entirety for all purposes. This pathway is independent of ATP synthesis (oligomycin-resistant), depends on permissive cytosolic Ca²⁺, is accompanied by smaller mitochondrial glutathione reduction, and is achieved by activating mitochondrial complex II. This is interesting taking into consideration that Gpx2 expression (glutathione peroxidase 2) (Table 7) and only one gene from Oxphos Complex II was reduced in the R138X mice. Correspondingly, deletion of the protein kinase Liver Kinase B1 (LKB1) selectively in beta cells leads to marked mitochondrial defects and impaired Ca²⁺ dynamics, while glucose-stimulated insulin secretion is enhanced most likely due to the upregulation of the latter pathway. See, e.g., Swisa et al. (2015) J. Biol. Chem. 290(34):20934-20946, herein incorporated by reference in its entirety for all purposes. Further experiments examining respiration and insulin secretion can be undertaken to investigate whether this alternative pathway is predominantly used in beta cells from R138X mice.

Restoration of beta-cell number is a key strategy for the development of novel T2D drugs. One of the biggest challenges to this approach is that factors that promote beta-cell replication in rodents have often failed to do so in human beta cells. However, our data in mice modeling the human SLC30A8 R138X mutation suggest that an increase in beta-cell number is one of the mechanisms underlying the T2D protective effects of some SLC30A8 mutations in humans. Therefore, understanding the mechanisms underlying the observed expansion of beta-cell area in R138X mice could lead to the identification of novel therapeutic targets with higher chance of translating into humans.

In conclusion, our data from the R138X mice offer for the first time an explanation as to why humans carrying the R138X mutation are protected against T2D. See, e.g., Flannick et al. (2014) Nat. Genet. 46(4):357-363, herein incorporated by reference in its entirety for all purposes. It therefore is a relevant model to study the mechanism underlying this protection. This is especially important given that increasing insulin secretion is a prominent therapeutic strategy to combat T2D. Therefore, understanding the mechanism underlying the increased insulin secretion in R138X mice could lead to the identification of novel therapeutic targets.

### Mechanism of Action

We have generated a new mouse model (R138X) mimicking the human SLC30A8 LoF mutation p.Arg138* that protects humans from the development of type 2 diabetes. R138X mice in high-fed-diet conditions have increased insulin secretion associated with increased beta-cell mass and proliferation. Alternatively, when we induce hyperglycemia using S961 (insulin receptor inhibitor), R138X mice have 50% increased insulin secretion that is independent from beta-cell mass and proliferation changes compared to WT mice. This suggests that the insulin receptor is required for the increase in proliferation in R138X mice. In addition, the insulin receptor in beta cells has been shown to be required for an increase in beta-cell proliferation in mice on high-fat diet. Since R138X mice have increased capacity to secrete insulin, the higher insulin can act on the insulin receptor in beta cells and induce proliferation to a larger extent than in WT mice. To investigate this, activation of the insulin pathway is examined by doing histology for p-Akt, p-S6, and p-Erk in pancreata from WT and R138X mice treated with high-fat diet or S961. In addition, isolated islets are examined to determine whether they have higher p-AKT and p-Erk levels and whether they proliferate more in vitro.

### Methods

Animals.

All procedures were conducted in compliance with protocols approved by the Regeneron Pharmaceuticals Institutional Animal Care and Use Committee. The Slc30a8 R138X and knockout mouse lines were made in pure C57BL/6NTac background using VELOCIGENE technology. See, e.g., Valenzuela et al. (2003) Nat. Biotechnol. 21(6):652-659, herein incorporated by reference in its entirety for all purposes. For the generation of the knockout mouse line, the whole Slc30a8 coding region was deleted and replaced with a LacZ gene. The neomycin gene was removed through the use of a self-deleting neomycin selection cassette. For the generation of the R138X mice, nucleotide 409 was changed from T into C in exon 3, which changes the arginine into a stop codon. The mutated allele has a self-deleting neomycin selection cassette flanked by loxP sites inserted at intron 3, deleting 29 bp of endogenous intron 3 sequence (CAGCTGACAGCAAGATTAATGGAAGTACC (SEQ ID NO: 24)). Mice were housed (up to five mice per cage) in a controlled environment (12-h light/dark cycle, 22±1° C., 60-70% humidity) and fed ad libitum with either chow (Purina Laboratory 23 Rodent Diet 5001, LabDiet) or high-fat diet (Research Diets, D12492; 60% fat by calories) starting at age 12-18 weeks. All data shown are compared to WT littermates.

Glucose Tolerance Test.

Mice were fasted overnight (16 hr) followed by oral gavage of glucose (Sigma) at 2 g/kg body weight. Blood samples were obtained from the tail vein at the indicated times and glucose levels were measured using the AlphaTrak2 glucometer (Abbott). Submandibular bleeds for insulin were done at 0, 15, and 30 min post-injection in separate experiments to not interfere with glucose measurements.

Insulin Tolerance Test.

Mice were fasted for 4 hr followed by intraperitoneal injection of either 0.75 U/kg (chow-fed) or 1.5 U/kg (high-fat-fed) of human insulin (Eli Lilly). Blood samples were obtained from the tail vein at the indicated times and glucose was measured using the AlphaTrak2 glucometer (Abbott).

Plasma Insulin, Proinsulin and C-peptide Measurements.

Submandibular bleeds of either overnight fasted or fed animals were done in the morning or following an oGTT. Insulin was analyzed with the mouse insulin ELISA (Mercodia). Proinsulin was analyzed with the mouse proinsulin ELISA (Mercodia). C-peptide was analyzed with the mouse C-peptide ELISA (ALPCO). All ELISAs were performed according to the manufacturer's instructions.

Histology.

Pancreata were fixed in 10% neutral buffered formalin solution for 48 h and then embedded in paraffin. Two sections of the pancreas from each animal were stained with an α-glucagon (REGN745, an α-glucagon monoclonal antibody generated in-house) or an α-insulin (Dako) antibody, and areas of glucagon and insulin positive cells were measured using Halo digital imaging analysis software (Indica Labs). The percent of glucagon and insulin positive areas in proportion to the whole pancreas area were calculated. The islet number per slide was counted and normalized to the whole pancreas area. The analysis was set to capture every cluster of insulin-positive cells down to a single cell resolution. We excluded artifacts (e.g., debris) smaller than one single beta cell (<150 μm²; area calculated using a diameter of about 14 μm). For fluorescence staining, pancreas sections were stained with a combination of α-insulin (Dako, A0564) antibody and α-KI67 antibody (R&D) followed by appropriate secondary antibodies. Fluorescent signal was detected using a microscope slide scanner (Zeiss Axio Scan.Z1). Islet cell types were quantified using the HALO image analysis using the Cytonuclear Fluorescence module (Indica Labs). The percent of glucagon- and insulin-positive areas relative to the whole pancreas area were calculated. Taking the area as basis, α-cell mass was calculated by multiplying the α-cell area for each animal by the weight of the animal's pancreas. To quantify islet mass, islet number and size was measured by counting the number of insulin-positive islets on a section. Every stained area larger than 150 μm was counted as an islet. The islet number was normalized to the whole pancreas area multiplied by the individual pancreas weight to get the islet mass.

RNA In Situ Hybridization.

For RNA analysis, pancreas tissue was permeabilized and hybridized with combinations of mRNA probes for mouse Gcg, Ins2, and Slc30A8 according to the manufacturer's instructions (Advanced Cell Diagnostics). A fluorescent kit was used to amplify the mRNA signal. Fluorescent signal was detected using a microscope slide scanner (Zeiss Axio Scan.Z1).

Islet Isolation and Dithizone Staining.

Mouse islets were isolated by density gradient separation after perfusing pancreas with Liberase TL (Roche, #05401020001) through the common bile duct. Following 13 min digestion at 37° C., the pancreas solution was washed and filtered through a 400-μm wire mesh strainer and islets were separated by Histopaque gradient centrifugation (Sigma-Aldrich, #H 1077) or by hand picking under a dissection microscope. Isolated islets were cultured overnight in RPMI-1640 medium (Gibco), supplemented with 10% (v/v) FBS, 10 mM HEPES, 50 μM β-mercaptoethanol, 1.0 mM sodium-pyruvate, 100 U/mL penicillin and 100 μg/mL streptomycin at 37° C. with a 5% CO₂ in air atmosphere. Up to 50 islets were hand-picked and transferred to a new dish containing 100 g/ml dithizone solution. Islets were stained for up to 10 min and transferred back into a PBS solution for microscopy (Zeiss Confocal microscope).

Western (Immuno-) Blot Analysis.

Islets were lysed in RIPA lysis buffer and the protein concentration was determined. 20 μg of total cell lysate was resolved by SDS/PAGE using Criterion TGX 4-20% precast gel (Bio-Rad) under reducing conditions and transferred to nitrocellulose membranes. The membranes were probed with a polyclonal α-mouse-SLC30A8 Rabbit Ab (custom made from Thermo Fisher) and detected using an enhanced chemiluminescent detection system. The SLC30A8 polyclonal Ab was raised against a peptide spanning amino acids 29-43 of the mouse SLC30A8 protein.

R138X Overexpression and Proteasomal Inhibition.

Human SLC30A8 WT and the first 137 amino acids followed by a stop codon were cloned with a C-terminal myc tag. HEK293 cells (0.4×10⁶ cells/well) were seeded into a 6-well plate. The next day, cells were transfected with 0.1 μg DNA/well using Mirus TransIT-TL1 following the manufacturer's instructions. Two days after transfection either DMSO, proteasomal (10 μM MG-132 or 1 μM Epoxomicin) or lysosomal inhibitors (10 μg/ml Cloroquine Salt) were spiked in. Cells were harvested after 6 h treatment in RIPA buffer. 30 μg of the cell lysates were analyzed by Western blotting.

qPCR Analysis.

Between 37.5 nanograms and 375 nanograms of RNA, per RT-qPCR assay to be run, was mixed with SuperScript® VILO™ Master Mix (ThermoFisher, Cat#11755500) and cycled according to the manufacturer's instructions. The cDNA was diluted with nuclease free water to between 0.5 nanograms per microliter and 5 nanograms per microliter. RT-qPCR assays were made by combining water, mastermix (ThermoFisher, Cat #4370074 or Bioline, Cat #CSA-01113), and 20× assay mix. The assay mix was a commercially available mixture of forward and reverse primers combined with a fluorescently labeled and quenched probe sequence (ThermoFisher, Cat#351372 or LGC BioSearch, Cat# DLO-RFBL-MIX). Samples were run in triplicate on a 384-well plate (ThermoFisher, Cat#4343370) by pipetting 5 microliters diluted cDNA and 10 microliters of appropriate RT-PCR assay into each well. The 384-well plate was covered with an optically clear seal (Agilent, Cat#16985-001), spun down, and read on an ABI 7900HT Fast Real-Time PCR System with 384-Well Block Module and Automation Accessory (ThermoFisher, Cat#4329002) for 40 cycles according to the specifics of the mastermix used. The sequences of the Slc30a8 probe and primers are as follows:

Probe:
(SEQ ID NO: 25)
TCCAAAACTGGGCAGTGAGTTCAACA,
Forward primer:
(SEQ ID NO: 26)
AATTGCAGTGCTGCTTTGC,
Reverse primer:
(SEQ ID NO: 27)
AGCTGCGGCTGTTGTTGTC.

RNA Preparation.

Islets were isolated as described above and incubated over night at 37° C. The next day, 500 islets per genotype were picked into Trizol (Invitrogen) as one sample and kept at −80° C. until RNA extraction and sequencing. Five different WT samples and four different R138× samples were analyzed. Total RNA was purified from all samples using MagMAX™-96 for Microarrays Total RNA Isolation Kit (Ambion by Life Technologies) according to the manufacturer's specifications. Genomic DNA was removed using MagMAX™Turbo™DNase Buffer and TURBO DNase from the MagMAX kit listed above (Ambion by Life Technologies). mRNA was purified from total RNA using Dynabeads® mRNA Purification Kit (Invitrogen). Strand-specific RNA-seq libraries were prepared using KAPA mRNA-Seq Library Preparation Kit (Kapa Biosystems). Twelve-cycle PCR was performed to amplify libraries. Sequencing was performed on Illumina HiSeq®2500 (Illumina) by multiplexed single-read run with 33 cycles.

RNAseq Read Mapping and Statistical Analysis of Differentially Expressed RNA.

Raw sequence data (BCL files) were converted to FASTQ format via Illumina bcl2fastq v2.17. Reads were decoded based on their barcodes and read quality was evaluated with FastQC. Reads were mapped to the mouse genome (NCBI GRCm38) using ArrayStudio® software (OmicSoft®) allowing two mismatches. Reads mapped to the exons of a gene were summed at the gene level. Differential expressed genes were identified by DESeq2 package and significantly perturbed genes were defined with fold changes no less than 1.5 in either up or down direction and with p-values of at least 0.01.

Data Analyses.

Data are reported as mean±SEM. Statistical analyses were performed using Prism 6.0 (GraphPad Software). All parameters were analyzed by two-way ANOVA (*p) and Students TTest (^{#}p) as indicated always comparing genotypes not treatments; a threshold of P<0.05 was considered statistically significant. Significant difference between genotypes by two-way ANOVA is indicated on the top of the graph. Significant differences of specific data points are indicated on top of the point/graph. *p/^{#}p<0.5, **p/^{##}p<0.01, ***p/^{###}p<0.001, ****p/^{####}p<0.0001.

1. A non-human animal whose genome comprises an endogenous Slc30a8 locus comprising a mutated Slc30a8 gene, wherein the mutated Slc30a8 gene encodes a truncated SLC30A8 protein and results in the non-human animal having an enhanced capacity for insulin secretion relative to a non-human animal without the mutation.

2. The non-human animal of claim 1, wherein the enhanced capacity for insulin secretion is in response to hyperglycemia.

3. The non-human animal of claim 2, wherein the non-human animal has increased insulin secretion in response to hyperglycemia induced by insulin receptor inhibition relative to the non-human animal without the mutation.

4. The non-human animal of claim 3, wherein the increased insulin secretion in response to hyperglycemia induced by insulin receptor inhibition is not associated with increased beta-cell proliferation or beta-cell mass relative to the non-human animal without the mutation.

5. The non-human animal of claim 1, wherein the enhanced capacity for insulin secretion is when fed a high-fat diet.

6. The non-human animal of claim 5, wherein the non-human animal has increased insulin secretion relative to the non-human animal without the mutation when fed a high-fat diet, wherein the increased insulin secretion is associated with increased beta-cell proliferation or beta-cell mass relative to the non-human animal without the mutation.

7. The non-human animal of claim 6, wherein the increased beta-cell proliferation is insulin-receptor-dependent.

8. The non-human animal of claim 1, wherein the mutated Slc30a8 gene has a premature termination codon.

9. The non-human animal of claim 1, wherein the mutated Slc30a8 gene comprises a mutation is in the third exon of the Slc30a8 gene.

10. The non-human animal of claim 9, wherein the mutation is at the 3′ end of the third exon of the Slc30a8 gene.

11. The non-human animal of claim 1, wherein the mutated Slc30a8 gene comprises a nonsense mutation.

12. The non-human animal of claim 11, wherein the nonsense mutation is in a codon corresponding to the codon encoding R138 in SEQ ID NO: 14 when the SLC30A8 protein encoded by the mutated Slc30a8 gene is optimally aligned with SEQ ID NO: 14.

13. The non-human animal of claim 11, wherein the nonsense mutation is at a position corresponding to residue 412 in SEQ ID NO: 21 when the coding sequence of the mutated Slc30a8 gene is optimally aligned with SEQ ID NO: 21.

14. The non-human animal of claim 1, wherein the mutated Slc30a8 gene is endogenous to the non-human animal.

15. The non-human animal of claim 1, wherein the non-human animal is a rat or a mouse.

16. The non-human animal of claim 15, wherein the non-human animal is a mouse.

17. The non-human animal of claim 16, wherein the mutated Slc30a8 gene encodes a SLC30A8 protein comprising the sequence set forth in SEQ ID NO: 13.

18. The non-human animal of claim 16, wherein the mutated Slc30a8 gene comprises the coding sequence set forth in SEQ ID NO: 22.

19. The non-human animal of claim 1, wherein the non-human animal has decreased mitochondrial gene expression relative to the non-human animal without the mutation.

20. The non-human animal of claim 1, wherein the non-human animal has increased Hvcn1 expression relative to the non-human animal without the mutation.

21. The non-human animal of claim 1, wherein the non-human animal has normal glucose homeostasis and glucose-induced insulin secretion on a control chow diet relative to the non-human animal without the mutation.

22. The non-human animal of claim 1, wherein the non-human animal has a normal metabolic phenotype on a control chow diet relative to the non-human animal without the mutation.

23. The non-human animal of claim 1, wherein the non-human animal has one or more of the following characteristics relative to the non-human animal without the mutation:
(a) increased glucose-induced insulin secretion when fed the high-fat diet;
(b) increased pancreatic beta-cell proliferation when fed the high-fat diet;
(c) increased number of pancreatic beta cells when fed the high-fat diet; and
(d) increased fed plasma insulin levels after blockade of the insulin receptor.

24. The non-human animal of claim 1, wherein the non-human animal has all of the following characteristics relative to the non-human animal without the mutation:
(a) increased glucose-induced insulin secretion when fed the high-fat diet;
(b) increased pancreatic beta-cell proliferation when fed the high-fat diet;
(c) increased number of pancreatic beta cells when fed the high-fat diet; and
(d) increased fed plasma insulin levels after blockade of the insulin receptor.

25. The non-human animal of claim 1, wherein the non-human animal has one or more of the following characteristics relative to the non-human animal without the mutation:
(a) increased circulating insulin levels after fed the high-fat diet for 20 weeks;
(b) increased number of pancreatic beta cells after fed the high-fat diet for 20 weeks;
(c) decreased proinsulin-to-insulin ratio when fed the high-fat diet; and
(d) increased fed plasma insulin levels after blockade of the insulin receptor.

26. The non-human animal of claim 1, wherein the non-human animal has all of the following characteristics relative to the non-human animal without the mutation:
(a) increased circulating insulin levels after fed the high-fat diet for 20 weeks;
(b) increased number of pancreatic beta cells after fed the high-fat diet for 20 weeks;
(c) decreased proinsulin-to-insulin ratio when fed the high-fat diet; and
(d) increased fed plasma insulin levels after blockade of the insulin receptor.

27. The non-human animal of claim 1, wherein Slc30a8 mRNA expression levels in the islets of the non-human animal are at least 25% of Slc30a8 mRNA expression levels in the islets of the non-human animal without the mutation.

28. The non-human animal of claim 1, wherein the non-human animal is heterozygous for the mutation.

29. The non-human animal of claim 1, wherein the non-human animal is homozygous for the mutation.

30. The non-human animal of claim 1, wherein the non-human animal is male.

31. The non-human animal of claim 1, wherein the non-human animal is female.

32. A method of making the non-human animal of claim 1, comprising:
(a) contacting the genome of a non-human animal pluripotent cell that is not a one-cell stage embryo with:
(i) an exogenous repair template comprising an insert nucleic acid flanked by a 5′ homology arm that hybridizes to a 5′ target sequence at the Slc30a8 locus and a 3′ homology arm that hybridizes to a 3′ target sequence at the Slc30a8 locus, wherein the insert nucleic acid comprises the mutation;
(ii) a Cas9 protein; and
(iii) a guide RNA that hybridizes to a guide RNA recognition sequence within the Slc30a8 locus,
wherein the Slc30a8 gene is modified to comprise the mutation; and
(b) introducing the modified non-human animal pluripotent cell into a host embryo; and
(c) implanting the host embryo into a surrogate mother to produce a genetically modified F0 generation non-human animal in which the Slc30a8 gene is modified to comprise the mutation, wherein the mutation results in the F0 generation non-human animal having an enhanced capacity for insulin secretion relative to a non-human animal without the mutation when fed the high-fat diet.

33. (canceled)

34. (canceled)

35. A method of making the non-human animal of claim 1, comprising:
(a) contacting the genome of a non-human animal one-cell stage embryo with:
(i) an exogenous repair template comprising an insert nucleic acid flanked by a 5′ homology arm that hybridizes to a 5′ target sequence at the Slc30a8 locus and a 3′ homology arm that hybridizes to a 3′ target sequence at the Slc30a8 locus, wherein the insert nucleic acid comprises the mutation;
(ii) a Cas9 protein; and
(iii) a guide RNA that hybridizes to a guide RNA recognition sequence within the Slc30a8 locus,
wherein the Slc30a8 gene is modified to comprise the mutation; and
(b) implanting the modified non-human animal one-cell stage embryo into a surrogate mother to produce a genetically modified F0 generation non-human animal in which the Slc30a8 gene is modified to comprise the mutation, wherein the mutation results in the F0 generation non-human animal having an enhanced capacity for insulin secretion relative to a non-human animal without the mutation when fed the high-fat diet.

36.-38. (canceled)

39. A method of screening a compound for activity for ameliorating or exacerbating type-2-diabetes, comprising:
(a) contacting a subject non-human animal of claim 1 with the compound; and
(b) measuring one or more of the following in the subject non-human animal relative to a control non-human animal not contacted with the compound, wherein the control non-human animal comprises the same Slc30a8 mutation as the subject non-human animal: (1) glucose-induced insulin secretion when fed a high-fat diet; (2) pancreatic beta-cell proliferation levels when fed the high-fat diet; (3) number of pancreatic beta cells when fed the high-fat diet; and (4) fed plasma insulin levels after blockade of the insulin receptor,
whereby activity for ameliorating type 2 diabetes is identified by one or more of the following in the subject non-human animal compared with the control non-human animal: (1) increased glucose-induced insulin secretion when fed the high-fat diet; (2) increased pancreatic beta-cell proliferation when fed the high-fat diet; (3) increased number of pancreatic beta cells when fed the high-fat diet; and (4) increased fed plasma insulin levels after blockade of the insulin receptor, and
whereby activity for exacerbating type 2 diabetes is identified by one or more of the following in the subject non-human animal compared with the control non-human animal: (1) decreased glucose-induced insulin secretion when fed the high-fat diet; (2) decreased pancreatic beta-cell proliferation when fed the high-fat diet; (3) decreased number of pancreatic beta cells when fed the high-fat diet; and (4) decreased fed plasma insulin levels after blockade of the insulin receptor.

40. A method of screening a compound for activity for ameliorating or exacerbating type-2-diabetes, comprising:
(a) contacting a subject non-human animal of claim 1 with the compound; and
(b) measuring one or more of the following in the subject non-human animal relative to a control non-human animal not contacted with the compound, wherein the control non-human animal comprises the same Slc30a8 mutation as the subject non-human animal: (1) capacity to secrete insulin in response to hyperglycemia; (2) insulin clearance; (3) mitochondrial gene expression; and (4) Hvcn1 expression,
whereby activity for ameliorating type 2 diabetes is identified by one or more of the following in the subject non-human animal compared with the control non-human animal: (1) increased capacity to secrete insulin in response to hyperglycemia; (2) increased insulin clearance; (3) decreased mitochondrial gene expression; and (4) increased Hvcn1 expression, and
whereby activity for exacerbating type 2 diabetes is identified by one or more of the following in the subject non-human animal compared with the control non-human animal: (1) decreased capacity to secrete insulin in response to hyperglycemia; (2) decreased insulin clearance; (3) increased mitochondrial gene expression; and (4) decreased Hvcn1 expression.

41. A non-human animal cell of the non-human animal of claim 1, wherein the genome of the non-human animal cell comprises an endogenous Slc30a8 locus comprising a mutated Slc30a8 gene, wherein the mutated Slc30a8 gene encodes a truncated SLC30A8 protein, and wherein a non-human animal comprising the mutated Slc30a8 gene has an enhanced capacity for insulin secretion relative to a non-human animal without the mutation.

42. (canceled)

43. A targeting vector for generating a mutated Slc30a8 gene at an endogenous Slc30a8 locus in a non-human animal, wherein the targeting vector comprises a 5′ homology arm targeting a 5′ target sequence at the endogenous Slc30a8 locus and a 3′ homology arm targeting a 3′ target sequence at the endogenous Slc30a8 locus, wherein the targeting vector comprises a mutation in the Slc30a8 gene, wherein the mutated Slc30a8 gene encodes a truncated SLC30A8 protein, and wherein a non-human animal comprising the mutated Slc30a8 gene has an enhanced capacity for insulin secretion relative to a non-human animal without the mutation.


SEQUENCE LISTING (27 sequences)

SEQ ID NO: 1 - DNA, 16 bp, Artificial Sequence
  Synthetic
cgaggcccccttccaa

SEQ ID NO: 2 - DNA, 17 bp, Artificial Sequence
  Synthetic
catttgggtggtatcga

SEQ ID NO: 3 - DNA, 22 bp, Artificial Sequence
  Synthetic
tgcgcaatcagagaatgttacc

SEQ ID NO: 4 - DNA, 17 bp, Artificial Sequence
  Synthetic
catttgggtggtattga

SEQ ID NO: 5 - DNA, 22 bp, Artificial Sequence
  Synthetic
tgtgggaatgtgagctctcaac

SEQ ID NO: 6 - DNA, 29 bp, Artificial Sequence
  Synthetic
cagctgacagcaagattaatggaagtacc

SEQ ID NO: 7 - DNA, 21 bp, Artificial Sequence
  Synthetic
gaccctggagacagaaatgtc

SEQ ID NO: 8 - DNA, 229 bp, Artificial Sequence
  Synthetic
  misc_feature (1)..(51): Slc30a8 intron 2
  misc_feature (52)..(198): Slc30a8 exon3 pArg137X
  misc_feature (199)..(229): Slc30a8 intron 3
cctaattgcattcccagcctttaacccagtgtgatattgtgcatctcacaggtggacacgttgctgggagtctggctatcctcactgatgcggctcatctcttaattgacctgactagtttcctgctcagtctcttttctttgtggttgtcatcgaggcccccttccaagcggctgacatttgggtggtattgagcaggtaacattctctgattgcgcaatagtgaatg

SEQ ID NO: 9 - DNA, 224 bp, Artificial Sequence
  Synthetic
  misc_feature (1)..(47): Slc30a8 intron 3
  misc_feature (48)..(53): XhoI
  misc_feature (54)..(87): loxP
  misc_feature (54)..(224): Self-deleting neomycin selection cassette
ctcaggataaaataaccatgtgggaatgtgagctctcaacattaattctcgagataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttacta

SEQ ID NO: 10 - DNA, 273 bp, Artificial Sequence
  Synthetic
  misc_feature (1)..(129): Self-deleting neomycin selection cassette
  misc_feature (90)..(123): loxP
  misc_feature (130)..(155): ICEUI
  misc_feature (156)..(161): NheI
  misc_feature (162)..(273): Slc30a8 intron 3
gatcagcagcctctgttccacatacacttcattctcagtattgttttgccaagttctaattccatcagacctcgacctgcagcccctagataacttcgtataatgtatgctatacgaagttatgctaggtaactataacggtcctaaggtagcgagctagctcctctactatgggacaacagaaatagctgtatcgtgatgccagacatttctgtctccagggtctaggagatgattctttcggttactctacatccttcgaagaaaggatga

SEQ ID NO: 11 - DNA, 424 bp, Artificial Sequence
  Synthetic
  misc_feature (1)..(137): Slc30a8 intron 3
  misc_feature (138)..(143): XhoI
  misc_feature (144)..(177): loxP
  misc_feature (184)..(209): ICEUI
  misc_feature (210)..(215): NheI
  misc_feature (216)..(424): Slc30a8 intron 3
atgatatagattcaaacagagcttgaaaaaaagggggaaggtaaagagaaagccttgcccaataattccctctgggctacaagcctgctgctcaggataaaataaccatgtgggaatgtgagctctcaacattaattctcgagataacttcgtataatgtatgctatacgaagttatgctaggtaactataacggtcctaaggtagcgagctagctcctctactatgggacaacagaaatagctgtatcgtgatgccagacatttctgtctccagggtctaggagatgattctttcggttactctacatccttcgaagaaaggatgaaggcagagaatctagggtttagtgattgtccctagggagttaggcataaaatatggactttcttaatcacacacatagtcattcctacgatctgaat

SEQ ID NO: 12 - protein, 367 aa, Mus musculus
MEFLERTYLVNDQATKMYAFPLDRELRQKPVNKDQCPGDRPEHPEAGGIYHCHNSAKATGNRSSKQAHAKWRLCAASAICFIFMVAEVVGGHVAGSLAILTDAAHLLIDLTSFLLSLFSLWLSSRPPSKRLTFGWYRAEILGALLSVLCIWVVTGVLLYLACERLLYPDYQIQAGIMITVSGCAVAANIVLTMILHQRNFGYNHKDVQANASVRAAFVHALGDVFQSISVLISALIIYFKPDYKIADPVCTFIFSILVLASTVMILKDFSILLMEGVPKGLSYNSVKEIILAVDGVISVHSLHIWSLTVNQVILSVHVATAASQDSQSVRTGIAQALSSFDLHSLTIQIESAADQDPSCLLCEDPQD

SEQ ID NO: 13 - protein, 136 aa, Artificial Sequence
  Synthetic
MEFLERTYLVNDQATKMYAFPLDRELRQKPVNKDQCPGDRPEHPEAGGIYHCHNSAKATGNRSSKQAHAKWRLCAASAICFIFMVAEVVGGHVAGSLAILTDAAHLLIDLTSFLLSLFSLWLSSRPPSKRLTFGWY

SEQ ID NO: 14 - protein, 369 aa, Homo sapiens
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 15 - protein, 137 aa, Homo sapiens
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWH

SEQ ID NO: 16 - DNA, 1906 bp, Mus musculus
atgtgcgcgcacgcgcgcgcgcacacacacacacacacacacacacacacacacacacacacacttattacaattgtacctttgagtctcccagaaaagcagtttctgtgagtgatagaatgagtctaggtgattttccaaggagactgttaattttcacagatttacccacaacactgatataggaggcctcattaactcaagtactggaagaatttttctaatttcttaggaagttgtgtgaatataataatatcagtgcttctttacttccaaaactggacagcgcatcaaacatcagaaacaacagtatcagctcctgtcccaactaccatggagtttcttgagagaacttatcttgtgaatgatcaagccaccaagatgtacgccttccctctagacagagaacttcgacagaagcctgtgaataaagatcagtgtcctggagacaggccagagcatccagaggcaggaggcatctatcactgccacaacagcgccaaggccacagggaacaggtcgagcaagcaagcgcatgccaagtggagactctgtgctgcttcagcaatatgcttcatctttatggtggcagaggtggtgggtggacacgttgctgggagtctggctatcctcactgatgcggctcatctcttaattgacctgactagtttcctgctcagtctcttttctttgtggttgtcatcgaggcccccttccaagcggctgacatttgggtggtatcgagcagagatcctcggtgccctgctgtctgtcctttgcatctgggtggtgactggtgtgctgctgtaccttgcctgtgagcgccttttgtatcctgattaccagatccaagcaggtatcatgatcactgtttcaggctgtgcagtggcagccaacattgtactaactatgattttgcaccaacggaactttggctacaaccacaaggatgtacaagctaatgccagtgtccgagcagcctttgtgcatgccctgggggatgtatttcagagcatcagtgtgctaattagtgctctcattatctactttaagcctgactacaaaattgctgatccagtgtgcacatttatcttttccatcctggttttggccagcaccgtcatgatcttaaaagacttctccatcttactcatggaaggtgttccaaagggcctgagttacaacagtgtgaaagagatcatcctcgcagttgatggcgtgatctccgtgcacagtctacacatctggtcactgactgtgaaccaagtgattctctctgttcatgttgctacagctgccagccaggacagccagtctgtgcggacaggaattgctcaagccctcagcagctttgatcttcactctcttaccattcagatagaatctgcagcagaccaggaccccagctgccttctctgcgaagaccctcaggactagctcggtcacactgtcagcttcctgtgtttcctaggccatgataagatgcagcaaagtttctgcaatgcacaatgaggcagccgtcggaatagatttgagaaagtcatgatgatgcaatgtgcacactcttcctttgtatttatctctatccaccatgaacgaggatgcatgggatttgtcggcttcttggattatacactaatcagtagttgtgctcaattgtagtatatatagattattcctaactggagctgaaataacagatgtttgcaatcataggtaatgaatgattcacttgcctacaatagtgggtatagttttactcggaaatgcctttctaggaatccacagcatgagaaacaaacatttgaagagaatttgaggccttagaatttgattctgccaccatactcaatgagatctttattttttgtgaaacagtaaaacttccctcttttgaccttgcatg

SEQ ID NO: 17 - DNA, 5373 bp, Homo sapiens
agcagtttttgtaggtgaaaacaatgaagccaggtaatattgcaaggaggctgtaattttagcagacctaccaacaacactgatgtaggaagctcattattttaatttctggagccttttaattttttctttagaaagtgtataaataattgcagtgctgctttgcttccaaaactgggcagtgagttcaacaacaacgacaacaacagccgcagctcatcctggccgtcatggagtttcttgaaagaacgtatcttgtgaatgataaagctgccaagatgtatgctttcacactagaaagtgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcaccgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactagctcagtcacaccgtcagtttcccaaatttgacaggccaccttcaaacatgctgctatgcagtttctgcatcatagaaaataaggaaccaaaggaagaaattcatgtcatggtgcaatgcacattttatctatttatttagttccattcaccatgaaggaagaggcactgagatccatcaatcaattggattatatactgatcagtagctgtgttcaattgcaggaatgtgtatatagattattcctgagtggagccgaagtaacagctgtttgtaactatcggcaataccaaattcatctcccttccaataatgcatcttgagaacacataggtaaatttgaactcaggaaagtcttactagaaatcagtggaagggacaaatagtcacaaaattttaccaaaacattagaaacaaaaaataaggagagccaagtcaggaataaaagtgactctgtatgctaacgccacattagaacttggttctctcaccaagctgtaatgtgatttttttttctactctgaattggaaatatgtatgaatatacagagaagtgcttacaactaatttttatttacttgtcacattttggcaataaatccctcttatttctaaattctaacttgtttatttcaaaactttatataatcactgttcaaaaggaaatattttcacctaccagagtgcttaaacactggcaccagccaaagaatgtggttgtagagacccagaagtcttcaagaacagccgacaaaaacattcgagttgaccccaccaagttgttgccacagataatttagatatttacctgcaagaaggaataaagcagatgcaaccaattcattcagtccacgagcatgatgtgagcactgctttgtgctagacattgggcttagcattgaaactataaagaggaatcagacgcagcaagtgcttctgtgttctggtagcaactcaacactatctgtggagagtaaactgaagatgtgcaggccaacattctggaaatcctatgtcaatgggtttggtttggaacctggacttctgcatttttaaaagttacccagagatgcttctaaagatgagccatagtctagaagattgtcaaccacaggagttcattgagtgggacagctagacacatacattggcagctacaatagtatcatgaattgcaatgatgtagtggggtataaaaggaaagcgatggatattgccggatgggcatggccagtgatgtttcacgtcattgaggtgacagctctgctggactttgaattacatatggaggctctccaggaagacgaagaagagaaggacattctaggcaaaaagaagactaggcacaaggcacacttatgtttgtctgttagcttttagttgaaaaagcaaaatacatgatgcaaagaaacctctccacgctgtgatttttaaaactacatactttttgcaactttatggttatgagtattgtagagaacaggagataggtcttagatgatttttatgttgttgtcagactctagcaaggtactagaaacctagcaggcattaataattgttgaggcaatgactctgaggctatatctgggccttgtcattatttatcatttatatttgtatttttttctgaaatttgagggccaagaaaacattgactttgactgaggaggtcacatctgtgccatctctgcaaatcaatcagcaccactgaaataactacttagcattctgctgagctttccctgctcagtagagacaaatatactcatcccccacctcagtgagcttgtttaggcaaccaggattagagctgctcaggttcccaacgtctcctgccacatcgggttctcaaaatggaaagaatggtttatgccaaatcacttttcctgtctgaaggaccactgaatggttttgtttttccatattttgcataggacgccctaaagactaggtgacttggcaaacacacaagtgttagtataattctttgcttctgcttctttttgaaaatcatgtttagatttgattttaagtcagaaattcactgaatgtcaggtaatcattatggagggagatttgtgtgtcaaccaaagtaattgtcccatggccccagggtatttctgttgtttccctgaaattctgcttttttagtcagctagattgaaaactctgaacagtagatgtttatatggcaaaatgcaagacaatctacaagggagattttaaggattttgagatgaaaaaacagatgctactcaggggctttatgaaccatccatcaattctgaagttctgactctcccattaccctttccctggtgtggtcagaactccaggtcactggaagttagtggaatcatgtagttgaattctttacttcaagacattgtattctctccagctatcaaaacattaatgatcttttatgtcttttttttgttattgttatactttaagttctggggtacatgtgcggaacatgtaggtttgttacataggtatacatgtgccatggtggtttgctgcactcatcaacctgtcatctacattcttttatgtctgtctttcaaagcaacactctgttcttctgagtagtgaaatcaggtcaactttaccaccagcctccatttttaatatgcttcaccatcatccagcacctacttaagatttatctagggctctgtggtgatgttaggacccataaaagaaatttatgccttccatatgtttggttacagatgggaaatgggaatgttgaaggacatgaaagaaaggatgtttacacattaagcatcagttctgaagctagattgtctgagtttgaatcttagctcttccctttattagctctgtgacctcgagctagttacttaaatgctctgatcctctatttcctgatcagtgaaacctccctattcaaatgtgtgagagtttaataaattaggacacttaaaaatgttggagcagtgcatagcatgtagtgttcagtacatgttaaatgttgttttttattatgtacaaacatgagtgggcacagaattttaaatcatctcaacttttgagaaattttgagttatcaacaccgttcccacaagacagtggcaaaattattggtgagaattaaacagctgtttctcagaggaagcaatggaggcttgctgggataaaggcatttactgagaggctgttacctagtgagagtgatgaattaattaaaatagtcgaatccctttctgactgtctctgaaagcttccgcttttatctttgaagagcagaattgtcactccaaggacatttattaataaaaagaacaactgtccagtgcaatgaaggcaaagtcataggtctcccaagtcttaccccattcctgtgaaatatcaagttcttggcttttctctgtcatgtagcctcaactttctctgaccgggtgcatttctttctctggtttctaaattgccagtggcaaatttggatcacttacttaatatctgttaaattttgtgacccaacaaagtcttttagcactgtggtgtcaaaaagaaaaacacctcccaggcatatacattttatagattcctggagaatgttgctctccagctccatccccacccaatgaaatatgatccagagagtcttgcaaagagacaagcctcattttccacaattagctctaaagtgcctccaggaaatgattttctcagctcatctctctgtattccctgttttggatcacagggcaatctgtttaaatgactaattacagaaatcattaaaggcaccaagcaaatgtcatctctgaatacacacatcccaagctttacaaatcctgcctggcttgacagtgatgaggccacttaacagtccagcgcaggcggatgttaaaaaaaataaaaaggtgaccatctgcggtttagttttttaactttctgatttcacacttaacgtctgtcattctgttactgggcacctgtttaaattctattttaaaatgttaatgtgtgttgtttaaaataaaatcaagaaagagaga

SEQ ID NO: 18 - DNA, 1906 bp, Artificial Sequence
  Synthetic
atgtgcgcgcacgcgcgcgcgcacacacacacacacacacacacacacacacacacacacacacttattacaattgtacctttgagtctcccagaaaagcagtttctgtgagtgatagaatgagtctaggtgattttccaaggagactgttaattttcacagatttacccacaacactgatataggaggcctcattaactcaagtactggaagaatttttctaatttcttaggaagttgtgtgaatataataatatcagtgcttctttacttccaaaactggacagcgcatcaaacatcagaaacaacagtatcagctcctgtcccaactaccatggagtttcttgagagaacttatcttgtgaatgatcaagccaccaagatgtacgccttccctctagacagagaacttcgacagaagcctgtgaataaagatcagtgtcctggagacaggccagagcatccagaggcaggaggcatctatcactgccacaacagcgccaaggccacagggaacaggtcgagcaagcaagcgcatgccaagtggagactctgtgctgcttcagcaatatgcttcatctttatggtggcagaggtggtgggtggacacgttgctgggagtctggctatcctcactgatgcggctcatctcttaattgacctgactagtttcctgctcagtctcttttctttgtggttgtcatcgaggcccccttccaagcggctgacatttgggtggtattgagcagagatcctcggtgccctgctgtctgtcctttgcatctgggtggtgactggtgtgctgctgtaccttgcctgtgagcgccttttgtatcctgattaccagatccaagcaggtatcatgatcactgtttcaggctgtgcagtggcagccaacattgtactaactatgattttgcaccaacggaactttggctacaaccacaaggatgtacaagctaatgccagtgtccgagcagcctttgtgcatgccctgggggatgtatttcagagcatcagtgtgctaattagtgctctcattatctactttaagcctgactacaaaattgctgatccagtgtgcacatttatcttttccatcctggttttggccagcaccgtcatgatcttaaaagacttctccatcttactcatggaaggtgttccaaagggcctgagttacaacagtgtgaaagagatcatcctcgcagttgatggcgtgatctccgtgcacagtctacacatctggtcactgactgtgaaccaagtgattctctctgttcatgttgctacagctgccagccaggacagccagtctgtgcggacaggaattgctcaagccctcagcagctttgatcttcactctcttaccattcagatagaatctgcagcagaccaggaccccagctgccttctctgcgaagaccctcaggactagctcggtcacactgtcagcttcctgtgtttcctaggccatgataagatgcagcaaagtttctgcaatgcacaatgaggcagccgtcggaatagatttgagaaagtcatgatgatgcaatgtgcacactcttcctttgtatttatctctatccaccatgaacgaggatgcatgggatttgtcggcttcttggattatacactaatcagtagttgtgctcaattgtagtatatatagattattcctaactggagctgaaataacagatgtttgcaatcataggtaatgaatgattcacttgcctacaatagtgggtatagttttactcggaaatgcctttctaggaatccacagcatgagaaacaaacatttgaagagaatttgaggccttagaatttgattctgccaccatactcaatgagatctttattttttgtgaaacagtaaaacttccctcttttgaccttgcatg

SEQ ID NO: 19 - DNA, 5373 bp, Homo sapiens
agcagtttttgtaggtgaaaacaatgaagccaggtaatattgcaaggaggctgtaattttagcagacctaccaacaacactgatgtaggaagctcattattttaatttctggagccttttaattttttctttagaaagtgtataaataattgcagtgctgctttgcttccaaaactgggcagtgagttcaacaacaacgacaacaacagccgcagctcatcctggccgtcatggagtttcttgaaagaacgtatcttgtgaatgataaagctgccaagatgtatgctttcacactagaaagtgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcactgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactagctcagtcacaccgtcagtttcccaaatttgacaggccaccttcaaacatgctgctatgcagtttctgcatcatagaaaataaggaaccaaaggaagaaattcatgtcatggtgcaatgcacattttatctatttatttagttccattcaccatgaaggaagaggcactgagatccatcaatcaattggattatatactgatcagtagctgtgttcaattgcaggaatgtgtatatagattattcctgagtggagccgaagtaacagctgtttgtaactatcggcaataccaaattcatctcccttccaataatgcatcttgagaacacataggtaaatttgaactcaggaaagtcttactagaaatcagtggaagggacaaatagtcacaaaattttaccaaaacattagaaacaaaaaataaggagagccaagtcaggaataaaagtgactctgtatgctaacgccacattagaacttggttctctcaccaagctgtaatgtgatttttttttctactctgaattggaaatatgtatgaatatacagagaagtgcttacaactaatttttatttacttgtcacattttggcaataaatccctcttatttctaaattctaacttgtttatttcaaaactttatataatcactgttcaaaaggaaatattttcacctaccagagtgcttaaacactggcaccagccaaagaatgtggttgtagagacccagaagtcttcaagaacagccgacaaaaacattcgagttgaccccaccaagttgttgccacagataatttagatatttacctgcaagaaggaataaagcagatgcaaccaattcattcagtccacgagcatgatgtgagcactgctttgtgctagacattgggcttagcattgaaactataaagaggaatcagacgcagcaagtgcttctgtgttctggtagcaactcaacactatctgtggagagtaaactgaagatgtgcaggccaacattctggaaatcctatgtcaatgggtttggtttggaacctggacttctgcatttttaaaagttacccagagatgcttctaaagatgagccatagtctagaagattgtcaaccacaggagttcattgagtgggacagctagacacatacattggcagctacaatagtatcatgaattgcaatgatgtagtggggtataaaaggaaagcgatggatattgccggatgggcatggccagtgatgtttcacgtcattgaggtgacagctctgctggactttgaattacatatggaggctctccaggaagacgaagaagagaaggacattctaggcaaaaagaagactaggcacaaggcacacttatgtttgtctgttagcttttagttgaaaaagcaaaatacatgatgcaaagaaacctctccacgctgtgatttttaaaactacatactttttgcaactttatggttatgagtattgtagagaacaggagataggtcttagatgatttttatgttgttgtcagactctagcaaggtactagaaacctagcaggcattaataattgttgaggcaatgactctgaggctatatctgggccttgtcattatttatcatttatatttgtatttttttctgaaatttgagggccaagaaaacattgactttgactgaggaggtcacatctgtgccatctctgcaaatcaatcagcaccactgaaataactacttagcattctgctgagctttccctgctcagtagagacaaatatactcatcccccacctcagtgagcttgtttaggcaaccaggattagagctgctcaggttcccaacgtctcctgccacatcgggttctcaaaatggaaagaatggtttatgccaaatcacttttcctgtctgaaggaccactgaatggttttgtttttccatattttgcataggacgccctaaagactaggtgacttggcaaacacacaagtgttagtataattctttgcttctgcttctttttgaaaatcatgtttagatttgattttaagtcagaaattcactgaatgtcaggtaatcattatggagggagatttgtgtgtcaaccaaagtaattgtcccatggccccagggtatttctgttgtttccctgaaattctgcttttttagtcagctagattgaaaactctgaacagtagatgtttatatggcaaaatgcaagacaatctacaagggagattttaaggattttgagatgaaaaaacagatgctactcaggggctttatgaaccatccatcaattctgaagttctgactctcccattaccctttccctggtgtggtcagaactccaggtcactggaagttagtggaatcatgtagttgaattctttacttcaagacattgtattctctccagctatcaaaacattaatgatcttttatgtcttttttttgttattgttatactttaagttctggggtacatgtgcggaacatgtaggtttgttacataggtatacatgtgccatggtggtttgctgcactcatcaacctgtcatctacattcttttatgtctgtctttcaaagcaacactctgttcttctgagtagtgaaatcaggtcaactttaccaccagcctccatttttaatatgcttcaccatcatccagcacctacttaagatttatctagggctctgtggtgatgttaggacccataaaagaaatttatgccttccatatgtttggttacagatgggaaatgggaatgttgaaggacatgaaagaaaggatgtttacacattaagcatcagttctgaagctagattgtctgagtttgaatcttagctcttccctttattagctctgtgacctcgagctagttacttaaatgctctgatcctctatttcctgatcagtgaaacctccctattcaaatgtgtgagagtttaataaattaggacacttaaaaatgttggagcagtgcatagcatgtagtgttcagtacatgttaaatgttgttttttattatgtacaaacatgagtgggcacagaattttaaatcatctcaacttttgagaaattttgagttatcaacaccgttcccacaagacagtggcaaaattattggtgagaattaaacagctgtttctcagaggaagcaatggaggcttgctgggataaaggcatttactgagaggctgttacctagtgagagtgatgaattaattaaaatagtcgaatccctttctgactgtctctgaaagcttccgcttttatctttgaagagcagaattgtcactccaaggacatttattaataaaaagaacaactgtccagtgcaatgaaggcaaagtcataggtctcccaagtcttaccccattcctgtgaaatatcaagttcttggcttttctctgtcatgtagcctcaactttctctgaccgggtgcatttctttctctggtttctaaattgccagtggcaaatttggatcacttacttaatatctgttaaattttgtgacccaacaaagtcttttagcactgtggtgtcaaaaagaaaaacacctcccaggcatatacattttatagattcctggagaatgttgctctccagctccatccccacccaatgaaatatgatccagagagtcttgcaaagagacaagcctcattttccacaattagctctaaagtgcctccaggaaatgattttctcagctcatctctctgtattccctgttttggatcacagggcaatctgtttaaatgactaattacagaaatcattaaaggcaccaagcaaatgtcatctctgaatacacacatcccaagctttacaaatcctgcctggcttgacagtgatgaggccacttaacagtccagcgcaggcggatgttaaaaaaaataaaaaggtgaccatctgcggtttagttttttaactttctgatttcacacttaacgtctgtcattctgttactgggcacctgtttaaattctattttaaaatgttaatgtgtgttgtttaaaataaaatcaagaaagagaga

SEQ ID NO: 20 - DNA, 1104 bp, Mus musculus
atggagtttcttgagagaacttatcttgtgaatgatcaagccaccaagatgtacgccttccctctagacagagaacttcgacagaagcctgtgaataaagatcagtgtcctggagacaggccagagcatccagaggcaggaggcatctatcactgccacaacagcgccaaggccacagggaacaggtcgagcaagcaagcgcatgccaagtggagactctgtgctgcttcagcaatatgcttcatctttatggtggcagaggtggtgggtggacacgttgctgggagtctggctatcctcactgatgcggctcatctcttaattgacctgactagtttcctgctcagtctcttttctttgtggttgtcatcgaggcccccttccaagcggctgacatttgggtggtatcgagcagagatcctcggtgccctgctgtctgtcctttgcatctgggtggtgactggtgtgctgctgtaccttgcctgtgagcgccttttgtatcctgattaccagatccaagcaggtatcatgatcactgtttcaggctgtgcagtggcagccaacattgtactaactatgattttgcaccaacggaactttggctacaaccacaaggatgtacaagctaatgccagtgtccgagcagcctttgtgcatgccctgggggatgtatttcagagcatcagtgtgctaattagtgctctcattatctactttaagcctgactacaaaattgctgatccagtgtgcacatttatcttttccatcctggttttggccagcaccgtcatgatcttaaaagacttctccatcttactcatggaaggtgttccaaagggcctgagttacaacagtgtgaaagagatcatcctcgcagttgatggcgtgatctccgtgcacagtctacacatctggtcactgactgtgaaccaagtgattctctctgttcatgttgctacagctgccagccaggacagccagtctgtgcggacaggaattgctcaagccctcagcagctttgatcttcactctcttaccattcagatagaatctgcagcagaccaggaccccagctgccttctctgcgaagaccctcaggactag

SEQ ID NO: 21 - DNA, 1110 bp, Homo sapiens
atggagtttcttgaaagaacgtatcttgtgaatgataaagctgccaagatgtatgctttcacactagaaagtgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcaccgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactag

SEQ ID NO: 22 - DNA, 1104 bp, Artificial Sequence
  Synthetic
atggagtttcttgagagaacttatcttgtgaatgatcaagccaccaagatgtacgccttccctctagacagagaacttcgacagaagcctgtgaataaagatcagtgtcctggagacaggccagagcatccagaggcaggaggcatctatcactgccacaacagcgccaaggccacagggaacaggtcgagcaagcaagcgcatgccaagtggagactctgtgctgcttcagcaatatgcttcatctttatggtggcagaggtggtgggtggacacgttgctgggagtctggctatcctcactgatgcggctcatctcttaattgacctgactagtttcctgctcagtctcttttctttgtggttgtcatcgaggcccccttccaagcggctgacatttgggtggtattgagcagagatcctcggtgccctgctgtctgtcctttgcatctgggtggtgactggtgtgctgctgtaccttgcctgtgagcgccttttgtatcctgattaccagatccaagcaggtatcatgatcactgtttcaggctgtgcagtggcagccaacattgtactaactatgattttgcaccaacggaactttggctacaaccacaaggatgtacaagctaatgccagtgtccgagcagcctttgtgcatgccctgggggatgtatttcagagcatcagtgtgctaattagtgctctcattatctactttaagcctgactacaaaattgctgatccagtgtgcacatttatcttttccatcctggttttggccagcaccgtcatgatcttaaaagacttctccatcttactcatggaaggtgttccaaagggcctgagttacaacagtgtgaaagagatcatcctcgcagttgatggcgtgatctccgtgcacagtctacacatctggtcactgactgtgaaccaagtgattctctctgttcatgttgctacagctgccagccaggacagccagtctgtgcggacaggaattgctcaagccctcagcagctttgatcttcactctcttaccattcagatagaatctgcagcagaccaggaccccagctgccttctctgcgaagaccctcaggactag

SEQ ID NO: 23 - DNA, 1110 bp, Homo sapiens
atggagtttcttgaaagaacgtatcttgtgaatgataaagctgccaagatgtatgctttcacactagaaagtgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcactgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactag

SEQ ID NO: 24 - DNA, 29 bp, Mus musculus
cagctgacagcaagattaatggaagtacc

SEQ ID NO: 25 - DNA, 26 bp, Artificial Sequence
  Synthetic
tccaaaactgggcagtgagttcaaca

SEQ ID NO: 26 - DNA, 19 bp, Artificial Sequence
  Synthetic
aattgcagtgctgctttgc

SEQ ID NO: 27 - DNA, 19 bp, Artificial Sequence
  Synthetic
agctgcggctgttgttgtc